(19) United States
(12) Patent Application Publication
(10) Pub. No.: US 2026/0022174 A1
(43) Pub. Date: Jan. 22, 2026

(54) Trispecific Antibodies for Activation of Immune Cells

(71) Applicant: JN BIOSCIENCES LLC, MOUNTAIN VIEW, CA (US)

(72) Inventors: Naoya Tsurushita, Mountain View, CA (US); J. Yun Tso, Mountain View, CA (US)

(21) Appl. No.: 19/272,198

(22) Filed: Jul. 17, 2025

Related U.S. Application Data
(60) Provisional application No. 63/672,635, filed on Jul. 17, 2024.

(51) Int. Cl. C07K 16/28; C07K 16/22

(52) U.S. Cl. CPC C07K 16/2818; C07K 16/22; C07K 16/2809; C07K 2317/24; C07K 2317/31; C07K 2317/565; C07K 2317/622; C07K 2317/71; C07K 2317/76

(57) ABSTRACT

The invention provides trispecific antibodies having one binding site binding to a target antigen on a cancer cell, pathogen, infected cell or autoreactive cell, and second and third binding sites binding to CD3 and CD28 respectively. Such antibodies can crosslink CD3 and CD28 on the surface of T cells at the cell-to-cell junction with target cells, and trigger CD3-mediated signal transduction (Signal 1) and costimulatory molecule-mediated signal transduction (Signal 2) for activation of T cells resulting in efficient elimination of target cells.

### CROSS-REFERENCE TO RELATED APPLICATION

This application claims the benefit of US 63/672,635, filed Jul. 17, 2024, which is incorporated by reference in its entirety for all purposes.

### SEQUENCE LISTING

The application includes sequences in an XML listing named 631059SEQLST.xml of 87,888 bytes created Jul. 16, 2025, which is incorporated by reference.

### BACKGROUND

Antigen-specific immune response by the adaptive immune system is a complex biological process that is controlled by multiple layers of positive and negative regulators. Naïve T cells are initially stimulated through the T cell receptor (TCR) complex, which comprises TCR α and β (or γ and δ) chains, CD3 gamma, delta, and epsilon molecules, and CD247 (also called CD3 zeta), by the recognition of their cognate peptide antigen presented by major histocompatibility complex (MHC; also called HLA for human proteins) on antigen-presenting cells (APCs). The initial interaction between TCR and MHC (or HLA) for T cell activation is referred to as Signal 1. Optimal activation and proliferation of naïve T cells requires a second signal provided by interaction of a costimulatory molecule CD28 expressed on T cells with their respective counter receptors CD80 and CD86 expressed on APCs (Signal 2). The immune system is further regulated positively by other costimulatory molecules such as ICOS that belong to the CD28 superfamily and CD40, OX40, GITR, CD27, HVEM and 4-1BB belonging to the TNF receptor superfamily. The immune system is also negatively regulated by checkpoint molecules such as PD-1, TIGIT, TIM-3, LAG-3, BTLA, VISTA, CD96 and CD112R. These costimulatory and checkpoint molecules are expressed in a cell type- and development stage-dependent manner to delicately control immune responses in the body. In addition, a variety of secreted proteins, such as cytokines and chemokines, are involved in regulation of immune responses by promoting activation, differentiation, proliferation, maintenance, and suppression of certain subsets of immune cells. The action of cytokines on T cells is often referred to as Signal 3, which is the third mechanism required for activation, differentiation, and proliferation of T cells. For reviews, see Curtsinger et al., Curr. Opin. Immunol. 22:333-340, 2010; Mahoney et al., Nat. Rev. Drug Discov. 14:561-584, 2015; Mercier et al., Front. Immunol. 6:418, 2015; Baumeister et al., Annu. Rev. Immunol. 34:539-573, 2016; Hurton et al., Proc. Natl. Acad. Sci. 113: E7788-E7797, 2016; Torphy et al., Int. J. Mol. Sci. 18:2642, 2017; Punt et al., Kuby Immunology, Eighth Edition. W. H. Freeman and Co., New York, 2018.

There are two major groups of mature T cells: CD4+ helper T cells and CD8+ cytotoxic T cells. CD4+ helper T cells are further divided into TH1, TH2, TH9, TH17, TH22, TFH, and Treg cells, each of which has specific functions and distinctive cytokine expression pattern. The main function of CD4+ helper T cells is to regulate other immune cells, such as B cells and CD8+ cytotoxic T cells, for proper and timely responses in the immune defense system. In contrast, the main function of CD8+ cytotoxic T cells is to destroy cells infected or transformed by pathogens in an antigen-specific manner. On activation by Signals 1 and 2, CD8+ cytotoxic T cells secrete perfolin and granzymes, which synergistically induce apoptosis of target cells. CD8+ cytotoxic T cells also play an important role in elimination of tumor cells. For reviews, see Taniuchi, Annu. Rev. Immunol. 36:579-601, 2018; Punt et al., supra; Saravia et al., Cell. Mol. Immunol. 16:634-643, 2019; Raskov et al., Br. J. Cancer 124:359-367, 2021.

Bispecific antibodies are engineered monoclonal antibodies that are capable of binding to two distinct antigens. Recent studies have reported that bispecific antibodies binding to CD3 expressed on T cells and a tumor-associated antigen (TAA) can bridge T cells to cancer cells, trigger crosslinking of CD3 on the surface of T cells and induce T cell-mediated cytotoxicity against such cancer cells. This type of bispecific antibodies is called a T cell engager. Several T cell engagers have been approved by the FDA for marketing as human therapeutics for treatment of cancer, including Blincyto (blinatumomab; anti-CD19/CD3) for treatment of acute lymphoblastic leukemia, Tecvayli (teclistamab; anti-BCMA/CD3) for multiple myeloma, Lunsumio (mosunetuzumab; anti-CD20/CD3) for follicular lymphoma, Columvi (glofitamab; anti-CD20/CD3) for diffuse large B-cell lymphoma, Talvey (talquetamab; anti-GPCR5/CD3) for multiple myeloma, and Elrexfio (elranatamab; anti-BCMA/CD3) for multiple myeloma. Many other bispecific T cell engagers are under evaluation in clinical studies for treatment of cancer. For reviews, see Middelburg et al., Cancers, 13:287, 2021; Ma et al., Front. Immunol. 12: Article 6266116, 2021; Wang et al., EMBO Mol. Med. 13: e14291, 2021; Arvedson et al., Annu. Rev. Cancer Biol. 6:17-34, 2022.

### SUMMARY OF THE CLAIMED INVENTION

The invention provides a trispecific antibody comprising first and second heavy chains and first and second instances of a light chain, wherein:
(a) the light chain comprises a light chain variable region and a light chain constant region;
(b) the first heavy chain comprises a first heavy chain variable region, a first heavy chain constant region and an scFv comprising a second heavy chain variable region and a second light chain variable region;
(c) the second heavy chain comprises the first heavy chain variable region, a second heavy chain constant region and an scFv comprising a third heavy chain variable region and a third light chain variable region;
(d) the first and second heavy chains respectively associate with the first and second instances of the light chain to form two instances of a first binding site, each instance comprising the first heavy chain variable region and first light chain variable region, the first binding site specifically binding to a target antigen;
(e) the scFv comprising the second heavy chain variable region and second light chain variable region forms a second binding site specifically binding to CD3;
(f) the scFv comprising the third heavy chain variable region and third light chain variable region forms a third binding site specifically binding to CD28; and
(g) the first and second heavy chains associate with one another via the first and second constant regions to form a tetramer of the first and second heavy chains and the first and second instances of the light chain.

Optionally the target is a cancer-associated antigen or an antigen on an infected cell, or an antigen on an autoreactive cell. Optionally, the first binding site antagonizes binding of the target to a ligand.

Optionally, the third heavy chain variable region comprises CDR H1 of SEQ ID NO:3, CDR H2 of SEQ ID NO:4 or 12 and CDR H3 of SEQ ID NO:5, and the third light chain variable region comprises CDRs L1, L2 and L3 of SEQ ID NOS:7-9 respectively. Optionally, the third heavy chain variable region and the third light chain variable region comprise SEQ ID NOS:78 and 79 respectively, or SEQ ID NOS:11 and 14 respectively. Optionally, the scFv forming the third binding site against CD28 comprises SEQ ID NO:28. Optionally, the second heavy chain variable region comprises CDRs H1, H2 and H3 of SEQ ID NOS:50-52 respectively and the second light chain variable region comprises CDRs L1, L2 and L3 of SEQ ID NOS:53-55 respectively. Optionally, the scFv forming the second binding site against CD3 comprises SEQ ID NO:32. Optionally, the first heavy chain variable region and first light chain variable region comprise residues 20-135 of SEQ ID NO:25 and residues 21-131 of SEQ ID NO:27 respectively. Optionally, the first heavy chain variable region and first light chain variable region comprise residues 20-138 of SEQ ID NO:35 and residues 21-127 of SEQ ID NO:37 respectively. Optionally, the first heavy chain variable region and the first light chain variable region comprises SEQ ID NO:43 and SEQ ID NO:46 respectively.

Optionally, the scFv forming the second binding site and the scFv forming the third binding site are linked via their light chain variable regions to the first and second heavy chain constant regions.

Optionally, the first and second heavy chains comprise SEQ ID NOS:30 and 33 respectively and the light chain comprises SEQ ID NO:31.

Optionally, the first, second and third binding sites are humanized, veneered or human and the heavy and light chain constant regions are human.

Optionally, the isotype of the first and second heavy chain constant regions is human IgG1 and the light chain constant region is human kappa.

Optionally, one or both of the heavy chain constant regions have at least one mutation reducing FcRγ binding.

Optionally one or both of the heavy chain constant regions have at least one mutation increasing binding to FcRn.

Optionally one of the first heavy chain constant region and second heavy chain constant region has at least one knob mutation and the other has at least one hole mutation to facilitate association of the first and second heavy chain constant regions.

The invention further provides a monoclonal antibody specifically binding to CD28 comprising a heavy chain variable region comprising CDR H1 of SEQ ID NO:3, CDR H2 of SEQ ID NO:4 or 12 and CDR H3 of SEQ ID NO:5, and a light chain variable region comprises CDRs L1, L2 and L3 of SEQ ID NOS: 7-9 respectively.

Optionally the monoclonal antibody comprises heavy and light chain variable regions of SEQ ID NOS:78 and 79 respectively, or SEQ ID NOS:11 and 14 respectively. Optionally, the monoclonal antibody is in the form of an scFv. Optionally, the monoclonal antibody comprises SEQ ID NO:28. Optionally, the first heavy chain variable region is linked to a heavy chain constant region and the first light chain variable region is linked to a light chain constant region. Optionally, the monoclonal antibody is in the form of a tetramer comprising two instances of the heavy chain and two instances of the light chain.

Optionally, the heavy chain variable region and the light chain variable region form a first binding site and the antibody further comprises a second binding site for a target other than CD28. Optionally, the target other than CD28 is a cancer-associated antigen, an antigen on an infected cell or an antigen on an autoreactive cell.

The invention further provides a pharmaceutical composition comprising a trispecific antibody or monoclonal antibody of any preceding claim and a pharmaceutically acceptable carrier.

The invention further provides a method of treating or effecting prophylaxis of cancer, comprising administering an effective regime of a trispecific antibody or monoclonal antibody as defined above to a subject having or at risk of cancer. Optionally, the cancer expresses the target antigen.

The invention further provides a method of treating an infection, comprising administering an effective regime of a trispecific antibody or monoclonal antibody as defined above to a subject having or at risk of infection. Optionally, the subject has infected cells comprising the target antigen.

### BRIEF DESCRIPTION OF THE DRAWINGS

FIGS. 1A-B: Amino acid sequence of mature HuTEC1 VH (SEQ ID NO:11) (A) and VL (SEQ ID NO:12) (B). Residue numbers are assigned according to Kabat et al. (Sequences of Proteins of Immunological Interest, National Institutes of Health, Bethesda, MD, 1987 and 1991). CDR sequences according to the definition of Kabat et al. (supra) are underlined.

FIG. 2: Nucleotide sequence of the HuTEC1 VH gene flanked by SpeI and HindIII sites (underlined) (SEQ ID NO:15) along with the deduced amino acid sequence (SEQ ID NO:10).

FIG. 3: Nucleotide sequence of the HuTEC1 VL gene flanked by NheI and EcoRI sites (underlined) (SEQ ID NO:16) along with the deduced amino acid sequence (SEQ ID NO:13).

FIGS. 4A-C: Schematic structures of expression vectors pHuTEC1.AA (A), pBS959 (B), and pJB564 (C).

FIG. 5: DNA sequence of the HuM195 VH gene flanked by SpeI and HindIII sites (underlined) (SEQ ID NO:24) along with the deduced amino acid sequence (SEQ ID NO:25).

FIG. 6: DNA sequence of the HuM195 VL gene flanked by NheI and EcoRI sites (underlined) (SEQ ID NO:26) along with the deduced amino acid sequence (SEQ ID NO:27).

FIG. 7: Schematic structure of bispecific and trispecific antibodies, BS959, JB564, YT423, JB559, YT419, JB554, YT405, and YT437 of this invention.

FIG. 8: Schematic structures of the expression vector pYT423.

FIG. 9: Schematic structures of the three types of antibodies expressed by pYT423.

FIG. 10: Fractionation of antibodies expressed from pYT423 by Mono S chromatography.

FIGS. 11A-B: T cell-mediated cytotoxicity by JB564 and YT423 against HL-60 cells (A) and THP-1 cells (B).

FIG. 12: DNA sequence of the 225 VH gene flanked by SpeI and HindIII sites (underlined) (SEQ ID NO:34) along with the deduced amino acid sequence (SEQ ID NO:35).

FIG. 13: DNA sequence of the 225 VL gene flanked by NheI and EcoRI sites (underlined) (SEQ ID NO:36) along with the deduced amino acid sequence (SEQ ID NO:37).

FIG. 14A-B: Cytotoxicity of JB559 and YT419 against HT-29 cells: Analysis of living HT-29 cells (A) and measurement of IFNγ in culture supernatants (B).

FIG. 15: DNA sequence of the C2B8 VH gene flanked by SpeI and HindIII sites (underlined) (SEQ ID NO:41) along with the deduced amino acid sequence (SEQ ID NO:42).

FIG. 16: DNA sequence of the C2B8 VL gene flanked by NheI and EcoRI sites (underlined) (SEQ ID NO:44) along with the deduced amino acid sequence (SEQ ID NO:45).

FIG. 17: Fractionation of antibodies expressed from pYT437 by Mono S chromatography.

### DEFINITIONS

Antibodies of the invention are typically provided in isolated form. This means that an antibody is typically at least 50% w/w pure of interfering proteins, including mispaired chains, and other contaminants arising from its production or purification but does not exclude the possibility that the trispecific antibody is combined with an excess of pharmaceutical acceptable carrier(s) or other vehicle intended to facilitate its use. Sometimes antibodies are at least 60, 70, 80, 90, 95 or 99% w/w pure of interfering proteins and contaminants from production or purification. Often an antibody is the predominant macromolecular species remaining after its purification.

Specific binding of an antibody to a target antigen means an affinity of at least 10⁶, 10⁷, 10⁸, 10⁹, or 10¹⁰ M⁻¹. Affinities can be different for different target antigens in a multi-specific antibody. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however necessarily imply that a trispecific antibody with three different binding sites binds only target antigens for these three binding sites. An antibody specifically binding a target antigen can also be described as being “against” or “directed to” the target antigen.

A basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. However, reference to a variable region does not mean that a signal sequence is necessarily present; and in fact signal sequences are cleaved once the antibodies of the invention have been expressed and secreted. A pair of heavy and light chain variable regions defines a binding region of an antibody. The carboxy-terminal portion of the light and heavy chains respectively defines light and heavy chain constant regions. The heavy chain constant region is primarily responsible for effector function. In IgG antibodies, the heavy chain constant region is divided into CH1, hinge, CH2, and CH3 regions. In IgA, the heavy chain constant region is divided into CH1, CH2 and CH3. The CH1 region binds to the light chain constant region by disulfide and noncovalent bonding. The hinge region provides flexibility between the binding and effector regions of an antibody and also provides sites for intermolecular disulfide bonding between the two heavy chain constant regions in a tetramer subunit. The CH2 and CH3 regions are the primary site of effector functions and FcRn binding.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” segment of about 12 or more amino acids, with the heavy chain also including a “D” segment of about 10 or more amino acids (see generally, Fundamental Immunology (Paul, W., ed., 2nd ed. Raven Press, N.Y., 1989), Ch. 7) (incorporated by reference in its entirety for all purposes).

The mature variable regions of each light/heavy chain pair form the antibody binding site. A natural antibody has two copies of the same binding site. A trispecific antibody has at least one copy of three different binding sites. In a preferred format, a trispecific antibody has two copies of a first binding site and single copies of second and third binding sites. The variable regions all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md., 1987 and 1991), or Chothia & Lesk, J. Mol. Biol. 196:901-917 (1987); Chothia et al., Nature 342:878-883 (1989), or other definition of CDRs as indicated in Table 1 below.

TABLE 1
Conventional Definitions of CDRs Using Kabat Numbering

| | | | Composite | | | |
|---|---|---|---|---|---|---|
| | | | of Chothia | | | |
| Loop | Kabat | Chothia | & Kabat | AbM | Contact | IMGT |
| L1 | L24--L34 | L24--L34 | L24--L34 | L24--L34 | L30--L36 | L27-32 |
| L2 | L50--L56 | L50--L56 | L50--L56 | L50--L56 | L46--L55 | L50-51 |
| L3 | L89--L97 | L89--L97 | L89--L97 | L89--L97 | L89--L96 | L89-97 |
| H1 | H31--H35B | H26-- | H26--H35B* | H26--H35B | H30--H35B | H26-H33 |
| | | H32 . . . H34* | | | | |
| H2 | H50--H65 | H52--H56 | H50--H65 | H50--H58 | H47--H58 | H51-H56 |
| H3 | H95--H102 | H95--H102 | H95--H102 | H95--H102 | H93--H101 | H93-102 |

*CDR-H1 by Chothia can end at H32, H33, or H34 (depending on the length of the loop). This is because the Kabat numbering scheme places insertions of extra residues at 35A and 35B, whereas Chothia numbering places them at 31A and 31B. If neither H35A nor H35B (Kabat numbering) is present, the Chothia CDR-H1 loop ends at H32. If only H35A is present, it ends at H33. If both H35A and H35B are present, it ends at H34.

Kabat also provides a widely used numbering convention (Kabat numbering) in which corresponding residues between different heavy chain variable regions or between different light chain variable regions are assigned the same number. Although Kabat numbering can be used for antibody constant regions, the Eu index (also called Eu numbering) is more commonly used, as is the case in this application.

The term “epitope” refers to a site on an antigen to which an antibody or binding site of a bispecific or trispecific antibody binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. Some antibodies bind to an end-specific epitope, meaning an antibody binds preferentially to a polypeptide with a free end relative to the same polypeptide fused to another polypeptide resulting in loss of the free end. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

The term “antigen” or “target antigen” or “target” indicates a target molecule bound by one binding site of an antibody. An antigen may be a protein of any length (natural, synthetic or recombinantly expressed), a nucleic acid or carbohydrate among other molecules. Antigens include receptors, ligands, counter receptors, and coat proteins. A cell bearing a target antigen can be referred to as a target cell.

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody bound to its antigen to identify contact residues. Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50:1495, 1990). A test antibody competes with a reference antibody if an excess of a test antibody (e.g., at least 2 times, 5 times, 10 times, 20 times or 100 times) inhibits binding of the reference antibody by at least 50% but preferably 75%, 90% or 99% as measured in a competitive binding assay. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

The term “subject” includes human and other mammalian subjects that receive either prophylactic or therapeutic treatment. Other mammalian subjects include animal models of a human condition (e.g., rodent, non-human primate) and veterinary subjects.

For purposes of classifying amino acids substitutions as conservative or nonconservative, amino acids are grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Percentage sequence identities are determined with antibody sequences maximally aligned by the Kabat numbering convention for a variable region or EU numbering for constant regions. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” one or more recited elements may include other elements not specifically recited. For example, a composition that comprises antibody may contain the antibody alone or in combination with other ingredients.

The term “antibody-dependent cellular cytotoxicity”, or ADCC, is a mechanism for inducing cell death that depends upon the interaction of antibody-coated target cells (i.e., cells with bound antibody) with immune cells possessing lytic activity (also referred to as effector cells). Such effector cells include natural killer cells, monocytes/macrophages and neutrophils. ADCC is triggered by interactions between the Fc region of an antibody bound to a cell and Fcγ receptors, particularly FcγRI and FcγRIII, on immune effector cells such as neutrophils, macrophages and natural killer cells. The target cell is eliminated by phagocytosis or lysis, depending on the type of mediating effector cell. Death of the antibody-coated target cell occurs as a result of effector cell activity.

The term opsonization also known as “antibody-dependent cellular phagocytosis”, or ADCP, refers to the process by which antibody-coated cells are internalized, either in whole or in part, by phagocytic immune cells (e.g., macrophages, neutrophils and dendritic cells) that bind to an immunoglobulin Fc region.

The term “complement-dependent cytotoxicity” or CDC (also called CMC) refers to a mechanism for inducing cell death in which an Fc effector domain(s) of a target-bound antibody activates a series of enzymatic reactions culminating in the formation of holes in the target cell membrane. Typically, antigen-antibody complexes such as those on antibody-coated target cells bind and activate complement component C1q which in turn activates the complement cascade leading to target cell death. Activation of complement may also result in deposition of complement components on the target cell surface that facilitate ADCC by binding complement receptors (e.g., CR3) on leukocytes.

### DETAILED DESCRIPTION

### I. General

Antigen presenting cell (APC)-derived malignant cells, such as B cell lymphoma and multiple myeloma, often express CD80 and/or CD86 which are counter receptors of CD28. Cell-to-cell interaction between such malignant cells and T cells promoted by an anti-CD3-based bispecific T cell engager, such as a bispecific anti-CD20/CD3 antibody, leads to crosslinking of CD3 at the cell-cell junction and triggering of CD3-mediated Signal 1 for activation of T cells. In addition, CD80 and/or CD86 molecules expressed in such malignant cells can cause crosslinking of CD28 molecules on T cells at the cell-cell junction, which triggers Signal 2 of T cell activation. With both Signals 1 and 2, activation and proliferation of T cells are induced efficiently, resulting in potent cytotoxicity against the malignant cells. However, if neither CD80 nor CD86 (nor other counter receptors of costimulatory molecules, such as OX40L, GITRL, CD70, Light, 4-1BBL, and ICOSL) are expressed on tumor cells, anti-TAA/CD3 bispecific antibodies do not efficiently activate T cells due to the lack of Signal 2.

The invention provides trispecific antibodies having one binding site binding to a target antigen on a cancer cell, infectious organism, infected cell or autoreactive immune cell, and second and third binding sites binding to CD3 and CD28 respectively. Although understanding of mechanism is not required for practice of the invention, it is believed that such trispecific antibodies can crosslink both CD3 and CD28 on the surface of T cells at the cell-to-cell junction with target cells, and trigger CD3-mediated signal transduction (Signal 1) and costimulatory molecule-mediated signal transduction (Signal 2) for activation of T cells, which can lead to efficient elimination of target cells. Thus, the immune cells can exert an immunotherapeutic effect against a cancer or other target cell or infectious organism with reduced toxicity to healthy tissue. The trispecific antibody can have additional activity against cells expressing a cancer-associated antigen as a result of antagonizing receptor interaction with a ligand (e.g., EGFR interaction with EGF, PD-L1 interaction with PD1 or CD33 interaction with sialic acid).

### II. Target Antigens

Trispecific antibodies of the invention have three different binding sites, a first binding site specifically binding to a target antigen, for example, a cancer-associated antigen, antigen on an infectious organism, or an antigen on an infected cell or an antigen on an autoreactive cell, and second and third binding sites specifically binding to CD3 and CD28.

Cancer-associated antigens are expressed by a cancer, typically at higher levels than control-matched normal tissue (overexpressed). Some examples of cancer-associated antigens are CD33, EGFR and PD-L1. Exemplary Swiss Prot numbers for human forms of these target antigens are P20138, P00533 and Q9NZQ7. CD33 binds sialic acid and is overexpressed primarily in cancers of myeloid origin, such as acute myeloid leukemia. EGFR binds EGF and is overexpressed primarily in gastric, breast, endometrial, colorectal cancer, head and neck cancer, ovarian, cervical, bladder and esophageal cancers. PD-L1 binds PD1 and is overexpressed in cancers such as gastric cancer, hepatocellular carcinoma, renal cell carcinoma, esophageal cancer, pancreatic cancer, ovarian cancer and bladder cancer.

Other examples of cancer-associated antigens include alpha-folate receptor (ovarian and epithelial cancers), CAIX (renal carcinoma), CD19 (B-cell malignancies, CLL, ALL), CD20 (B-cell malignancies, lymphomas), CD22 (B-cell malignancies), CD23 (CLL), CD24 (pancreatic CA), CD30 (lymphomas), CD33 (AML), CD38 (NHL), CD44v7/8 (cervical CA), CEA (colorectal CA), EGFRvIII (glioblastoma), EGP-2 (multiple malignancies), EGP-40 (colorectal CA), EphA2 (glioblastoma), Erb-B2 (breast, prostate, colon CA), FBP (ovarian CA), G.sub.D2 (neuroblastoma, melanoma), GD3 (melanoma), HER2 (pancreatic CA, ovarian CA, glioblastoma, osteosarcoma), HMW-MAA (melanoma), IL-11Rα (osteosarcoma), IL-13Rα2 (glioma, glioblastoma), KDR (tumor vasculature), kappa-light chain (B-cell malignancies), Lewis Y (various carcinomas), L1 (neuroblastoma), MAGE-A1 (melanoma), mesothelin (mesothelioma), MUC1 (breast and ovarian CA), MUC16 (ovarian CA), NKG2D (myeloma, ovarian CA), NY-ESO-1 (multiple myeloma), oncofetal antigen (various tumors), PSCA (prostate CA), PSMA (prostate CA), ROR1 (B-CLL), TAG-72 (adenocarcinomas), and VEGF-R2 (tumor neovasculature). (Sadelain et al., Cancer Discov 3:388-98, 2013). Other tumor-associated antigens that can be targeted include alpha-fetoprotein (AFP), alpha-actinin-4, A3, ART-4, B7, Ba 733, BAGE, BCMA, BrE3-antigen, CA125, CAMEL, CAP-1, carbonic anhydrase IX, CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD47, CD52, CD54, CD55, CD59, CD64, CD66a-e, CD67, CD70, CD70L, CD74, CD79a, CD79b, CD80, CD83, CD95, CD123,CD126, CD132, CD133, CD138, CD147, CD154, CD155,CDC27, CDK-4/m, CDKN2A, CTLA4, CXCR4, CXCR7, CXCL12, HIF-1α, colon-specific antigen-p (CSAp), CEA (CEACAM-5), CEACAM-6, c-Met, claudin 6, claudin 18.2, DAM, DLL3, EGFR, EGFRvIII, EGP-1 (TROP-2), EGP-2, ELF2-M, Ep-CAM, EphA10, fibroblast growth factor (FGF), Flt-1, Flt-3, folate receptor, G250 antigen, GAGE, GD2, gpA33, GPC3, gp100, GRO-beta, GUCY2C, HLA-DR, HLA-A*02:01:gp100,HM1.24, human chorionic gonadotropin (HCG) and its subunits, HER2/neu, HMGB-1, hypoxia inducible factor (HIF-1), HSP70-2M, HST-2, IGF-IR, IFN-γ, IFN-, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), integrin beta 4, KC4-antigen, KS-1-antigen, KS1-4, Le-Y, LDR/FUT, macrophage migration inhibitory factor (MIF), MAGE, MAGE-3, MART-1, MART-2, NY-ESO-1, TRAG-3, mCRP, MCP-1, MIP-1A, MIP-1B, MIF, MUC1, MUC2, MUC3, MUC4, MUC5ac, MUC13, MUC16, MUC17, MUM-1/2, MUM-3, NCA66, NCA95, NCA90, NY-ESO1 pancreatic cancer mucin, p-cadherin, PD1 receptor, placental growth factor, p53, PLAGL2, prolactin receptor, prostatic acid phosphatase, PSA, PSCA, PRAME, PSMA, PIGF, ILGF, ILGF-R, IL-6, IL-25, RORI, RS5, RANTES, T101, SAGE, S100, SSTR2, STEAP1, survivin, survivin-2B, TAC, TAG-72, tenascin, TRAIL receptors, transferrin receptor, TNF-α, Tn antigen, 5T4,Thomson-Friedenreich antigens, tumor necrosis antigens, VEGFR, ED-B fibronectin, WT-1, 17-1A-antigen, complement factors C3, C3a, C3b, C5a, C5, an angiogenesis marker, bcl-2, bcl-6, Kras, an oncogene marker or an oncogene product (see, e.g., Sensi et al., Clin Cancer Res 2006, 12:5023-32; Parmiani et al., J Immunol 2007, 178:1975-79; Novellino et al. Cancer Immunol Immunother. 2005, 54:187-207. CD33 binds sialic acid and is overexpressed primarily in cancers of myeloid origin, such as acute myeloid leukemia. EGFR binds EGF and is overexpressed primarily in gastric, breast, endometrial, colorectal cancer, head and neck cancer, ovarian, cervical, bladder and esophageal cancers. PD-L1 binds PD1 and is overexpressed in cancers such as gastric cancer, hepatocellular carcinoma, renal cell carcinoma, esophageal cancer, pancreatic cancer, ovarian cancer and bladder cancer.

Another class of proteins are antigens expressed on the surface of pathogens or pathogen-infected cells. Another class of proteins are antigens expressed on autoreactive immune cells associated with disease, such as autoimmune diseases. Autoreactive cells include B and T cells.

The other two binding sites bind to CD3 and CD28 respectively. Human CD3 is a complex including CD3 delta (e.g., Swiss Prot P04234), CD3 gamma (e.g., Swiss Prot PO9693), two molecules of CD3 epsilon (e.g., Swiss Prot P07766) and two molecules of CD3 zeta (e.g., Swiss Prot P20963). Reference to human CD3 and its subunits includes the exemplified human forms and other known allelic variants in humans as indicated in the Swiss Prot database. Antibodies binding to the epsilon component of CD3 are preferred. The Swiss Prot number for human CD28 is P10747. Unless otherwise apparent from the context, reference to a specific target should be understood as referring to human forms. However, non-human forms, such as those of laboratory (e.g., mouse, rat), non-human primates, companion animals or farm animals, can also be used.

### III. Exemplary Antibodies for Each Target Antigen

Trispecific antibodies are formed from pairs of heavy and light chain variable regions from component antibodies. The component antibodies can be rodent, chimeric, veneered, humanized, primatized, primate or human among others. The component antibodies can be of the same or different types; for example, one can be humanized and the other human.

The production of other non-human monoclonal antibodies, e.g., murine, guinea pig, primate, rabbit or rat, specifically binding to an antigen can be accomplished by, for example, immunizing the animal with the antigen or a fragment thereof, or cells bearing the antigen. See Harlow & Lane, Antibodies, A Laboratory Manual (CSHP NY, 1988) (incorporated by reference for all purposes). Such an antigen can be obtained from a natural source, by peptide synthesis or by recombinant expression. Optionally, the antigen can be administered fused or otherwise complexed with a carrier protein. Optionally, the antigen can be administered with an adjuvant. Several types of adjuvant can be used as described below. Complete Freund's adjuvant followed by incomplete adjuvant is preferred for immunization of laboratory animals.

A humanized antibody is a genetically engineered antibody in which the CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539, Carter, U.S. Pat. No. 6,407,213, Adair, U.S. Pat. Nos. 5,859,205 and 6,881,557, Foote, U.S. Pat. No. 6,881,557). The acceptor antibody sequences can be, for example, a mature human antibody sequence, a composite of such sequences, a consensus sequence of human antibody sequences, or a germline region sequence. Thus, a humanized antibody is an antibody having some or all CDRs entirely or substantially from a donor antibody and variable region framework sequences and constant regions, if present, entirely or substantially from human antibody sequences. Similarly a humanized heavy chain has at least one, two and usually all three CDRs entirely or substantially from a donor antibody heavy chain, and a heavy chain variable region framework sequence and heavy chain constant region, if present, substantially from human heavy chain variable region framework and constant region sequences. Similarly a humanized light chain has at least one, two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A CDR in a humanized antibody is substantially from a corresponding CDR in a non-human antibody when at least 85%, 90%, 95% or 100% of corresponding residues (as defined by Kabat) are identical between the respective CDRs. The variable region framework sequences of an antibody chain or the constant region of an antibody chain are substantially from a human variable region framework sequence or human constant region respectively when at least 85%, 90%, 95% or 100% of corresponding residues defined by Kabat are identical.

Although humanized antibodies often incorporate all six CDRs (preferably as defined by Kabat) from a mouse antibody, they can also be made with less than all CDRs (e.g., at least 3, 4, or 5 CDRs from a mouse antibody) (e.g., Pascalis et al., J. Immunol. 169:3076, 2002; Vajdos et al., Journal of Molecular Biology, 320:415-428, 2002; Iwahashi et al., Mol. Immunol. 36:1079-1091, 1999; Tamura et al, Journal of Immunology, 164:1432-1441, 2000).

A chimeric antibody is an antibody in which the mature variable regions of light and heavy chains of a non-human antibody (e.g., a mouse) are combined with human light and heavy chain constant regions. Such antibodies substantially or entirely retain the binding specificity of the mouse antibody and are about two-thirds human sequence.

A veneered antibody is a type of humanized antibody that retains some and usually all of the CDRs and some of the non-human variable region framework residues of a non-human antibody but replaces other variable region framework residues that may contribute to B- or T-cell epitopes, for example exposed residues (Padlan, Mol. Immunol. 28:489, 1991) with residues from the corresponding positions of a human antibody sequence. The result is an antibody in which the CDRs are entirely or substantially from a non-human antibody and the variable region frameworks of the non-human antibody are made more human-like by the substitutions.

A human antibody can be isolated from a human, or otherwise result from expression of human immunoglobulin genes (e.g., in a transgenic mouse, in vitro or by phage display). Methods for producing human antibodies include the trioma method of Oestberg et al., Hybridoma 2:361-367 (1983); Oestberg, U.S. Pat. No. 4,634,664; and Engleman et al., U.S. Pat. No. 4,634,666, use of transgenic mice including human immunoglobulin genes (see, e.g., Lonberg et al., WO93/12227 (1993); U.S. Pat. Nos. 5,877,397, 5,874,299, 5,814,318, 5,789,650, 5,770,429, 5,661,016, 5,633,425, 5,625,126, 5,569,825, 5,545,806, Nature 148, 1547-1553 (1994), Nature Biotechnology 14, 826 (1996), Kucherlapati, WO 91/10741 (1991)) and phage display methods (see, e.g. Dower et al., WO 91/17271 and McCafferty et al., WO 92/01047, U.S. Pat. Nos. 5,877,218, 5,871,907, 5,858,657, 5,837,242, 5,733,743 and 5,565,332).

Antibodies are screened for specific binding to the antigen. Antibodies may be further screened for binding to a specific region of the antigen, competition with a reference antibody, agonism or antagonism of cells bearing the antigen. Non-human antibodies can be converted to chimeric, veneered or humanized forms as described above.

The specification discloses exemplary antibodies specifically binding to CD33, EGFR, CD20, CD3 and CD28. These antibodies are characterized by the mature heavy and light chain variable region sequences and Kabat CDRs provided in the sequence listing as indicated in Table 2 below.

TABLE 2

| | Antibody | Mature | Mature | Kabat CDRs | Kabat CDRs |
|---|---|---|---|---|---|
| Target | name | VH | VL | H1, H2, H3 | L1, L2, L3 |
| CD28 | TEC1 | SEQ ID | SEQ ID | SEQ ID | SEQ ID |
| | | NO: 78 | NO: 79 | NOS: 3, | NOS: 7, |
| | | | | 4, 5 | 8, 9 |
| CD28 | HuTEC1 | SEQ ID | SEQ ID | SEQ ID | SEQ ID |
| | | NO: 11 | NO: 14 | NOS: 3, | NOS: 7, |
| | | | | 12, 5 | 8, 9 |
| CD3 | SP34 | SEQ ID | SEQ ID | SEQ ID | SEQ ID |
| | | NO: 56 | NO: 57 | NOS: 50, | NOS: 53, |
| | | | | 51, 52 | 54, 55 |
| CD3 | HuSP34V | SEQ ID | SEQ ID | SEQ ID | SEQ ID |
| | | NO: 58 | NO: 59 | NOS: 50, | NOS: 53, |
| | | | | 51, 52 | 54, 55 |
| CD33 | HuM195 | 20 to 135 | 21 to 131 | SEQ ID | SEQ ID |
| | | of SEQ ID | of SEQ ID | NOS: 60, | NOS: 63, |
| | | NO: 25 | NO: 27 | 61, 62 | 64, 65 |
| EGFR | 225 | 20 to 138 | 21 to 127 | SEQ ID | SEQ ID |
| | | of SEQ ID | of SEQ ID | NOS: 66, | NOS: 69, |
| | | NO: 35 | NO: 37 | 67, 68 | 70, 71 |
| CD20 | C2B8 | SEQ ID | SEQ ID | SEQ ID | SEQ ID |
| | | NO: 43 | NO: 46 | NOS: 72, | NOS: 75, |
| | | | | 73, 74 | 76, 77 |

CDR H2 of HuTEC1 differs from CDR H2 of TEC1 by replacement of Asp with Ser at the fifth position of the CDR (position 54 by Kabat numbering) to avoid potential aspartate isomerization (Lu et al., mAbs 11:45-57, 2019). Variable region framework amino acids likely to affect proper formation of the antigen binding site of TEC1 were residues at positions 30 and 49 in VH. These positions are preferably occupied by amino acids threonine and glycine at positions 30 and 49, respectively, in humanized TEC1. The specification discloses exemplary single-chain Fv (scFv) antibodies binding to CD3 and CD28. These scFv antibodies are listed in Table 3 below.

TABLE 3

| Target | Name of scFv | SEQ ID NO |
|---|---|---|
| CD3 | HuSP34V.scFv.ds | 32 |
| CD28 | HuTEC1.scFv.ds | 28 |

The HuTEC1.scFv.ds includes cysteine for alanine at heavy chain position 44 and cysteine for glycine at light chain position 100 (Kabat numbering). HuSP34V.scFv.ds includes cysteine for glycine at heavy chain position 44 and cysteine for glycine at light chain position 100 (Kabat numbering). Cysteines at position 44 in VH and position 100 in VL form a disulfide bond between VH and VL to stabilize their association for formation of the antigen-binding site in the format of scFv.

Other antibodies having the same CDRs as defined by Kabat, or alternative definitions, such as Chothia, composite of Chothia and Kabat, AbM or Contact (see world wide web bioinf.org.uk/abs) or binding to the same epitope or competing for binding with any of these antibodies to their target antigen can also be used. Other means for binding to any of the above targets can also be used in place of the above antibodies. Antibodies binding to CD3 or CD28 agonize their target antigen when placed in the trispecific format of this invention and thereby activate immune cells expressing the target. Antibodies binding to a cancer or pathogen associated antigen may or may not antagonize the interaction of the cancer-associated antigen with its ligand or counterreceptor. Antagonism provides an additional mechanism to suppress a cancer cell or pathogenic cell but is not necessary for activation of immune cells by a trispecific antibody binding to CD3 and CD28.

Antibodies specifically binding to CD33 include gentuzumab, lintuzumab, and BI836858 (Heider Blood 2011; 118(15):4159-4168). Antibodies specifically binding to EGFR include C225, a chimeric version of which is marketed as cetuximab, panitumumab, matuzumab and necitumumab. Antibodies specifically binding to CD20 include rituximab, ofatumumab, obinutuzumab, ocrelizumab, ublituximab. Bispecific antibodies binding to CD20 and CD3 include mosunetuzumab, glofitamab, epcoritamab, and odronextamab. Other antibodies specifically binding to CD3 include otelixizumab, teplizumab, visilizumab, and muromonab. Other agonistic antibodies specifically binding to CD28 include JJ316, D665, 5.11A1, TBH1412, 37.51, E18, PV-1, JJ319, PV1-IgG3, and FK734 (see Poirier et al., Am Jp. Transplantation 12-1682-1690 (2012), Table 1).

Any of these antibodies or other antibodies having the same CDRs as defined by Kabat, or alternative definitions, such as Chothia, composite of Chothia and Kabat, AbM or Contact, binding to the same epitope, or competing for binding with any of these antibodies to their target antigen can also be incorporated into the trispecific antibodies of the invention. Other means for binding to any of the above target antigens can also be used in place of the above antibodies.

The invention also provides monoclonal antibodies specifically binding to human CD28 and other antibodies sharing the same six CDRs (by any conventional definition) as one of these antibodies or the same pair of mature heavy and light chain variable regions as monoclonal antibodies. Such antibodies may or may not inhibit CD28 interaction with one or more of its ligands, CD80, CD86 and ICOS-L.

### IV. Formats for Trispecific Antibodies

Over 100 formats have been described for multi-specific antibodies (e.g., Kontermann et al., Drug Discovery Today 20, 838-847 (2015); Sedykh et al., Drug Des. Devel. Ther. 2, 195-209 (2018)). Formats for bispecific antibodies, which include at least one binding site for each of two targets, can be modified to be trispecific, for example, by inclusion of an scFv or single-domain antibody (sdAb) such as VHH, or fusion of scFv or sdAb at the terminus of one (or more) of the chains of a bispecific antibody.

A preferred format, which is followed in the examples of the present invention, is to incorporate second and third binding specificities into an IgG antibody by linking scFv's providing these specificities to the C-termini of each heavy chain constant region in an otherwise standard antibody providing the first binding site. Such a trispecific antibody includes first and second heavy chains and two instances of the same light chain. The light chain comprises from N to C terminus, a light chain variable region and a light chain constant region as in a standard antibody. The first heavy chain comprises from N to C terminus, a first heavy chain variable region, a first heavy chain constant region and a first scFv comprising a second heavy chain variable region and a second light chain variable region. The second heavy chain comprises from N to C terminus, the first heavy chain variable region, a second heavy chain constant region, the same or different from the first heavy chain constant region, and a second scFv comprising a third heavy chain variable region and a third light chain variable region. The first and second heavy chains associate with the first and second instances of the light chain by pairing of CH1 domains of the heavy chain constant regions and the light chain constant regions to form first and second instances of a first binding site comprising the first heavy chain variable region and light chain variable region. The first and second scFv's provide the second and third binding sites respectively. The first and second heavy chains associate with each other via pairing of their CH2 and/or CH3 regions and optionally intermolecular disulfide bonding between respective hinge regions. The result is a tetrameric molecule comprising first and second heavy chains associated with first and second instances of the light chain. The tetrameric format is similar to that of a normal antibody except for the presence of the scFv regions linked to the C-termini of the respective heavy chains. The scFvs can be linked independently of one another via either their light chain variable region or heavy chain variable region to the C-termini of the respective heavy chains. Linkage via the light chain variable regions is preferred.

In the above format, the first binding site preferably specifically binds to a target antigen, such as a cancer-associated antigen, antigen on an infectious organism or infected cells, or an antigen on an autoreactive cell. The second and third binding sites preferably specifically bind to CD3 and CD28. It is arbitrary whether the second binding site is directed against CD3 and the third against CD28 or vice versa.

The above format has the advantage of allowing binding to a target antigen in a format similar to that of a normal antibody. It also allows wide spatial separation of the second and third binding sites from the first binding site to facilitate bridging between a target cell and effector cell. It also presents the second and third binding site in the same conformation to an effector cell facilitating mutual binding of these sites to the effector cell.

Alternative but less preferred formats include attachment of scFv's against CD3 and CD28 respectively to the C-terminus of each light chain, in which case a trispecific antibody includes two different light chains and two instances of a heavy chain. In a further alternative, an scFv against CD3 can be attached to the C-terminus of each heavy (or light) chain and an scFv against CD28 attached to the C-terminus of each light (or heavy) chain, in which case a trispecific antibody includes two identical heavy chains and two identical light chains.

An scFv can be attached either through its light chain variable region or heavy chain variable region end to a CH3 region of a heavy chain constant region. Attachment can be direct or via a linker peptide. Usually the C-terminal lysines of the CH3 region is omitted with attachment to the adjacent, i.e., penultimate, residue glycine. When assembled by complexing of two paired heavy and light chains, such a trispecific antibody includes two copies of first binding site, and single copies of each of second and third binding sites.

Pairwise association of the first and second heavy chains with each other as opposed to dimerization of the first heavy chain with itself or second heavy chain with itself can be promoted by a “knobs-into-holes” approach involving substituting a large amino acid for a small one in the CH3 domain (the “knob”) of one heavy chain and vice versa (the “hole”) of the other heavy chain (Ridgway et al., Protein Eng 9:617-21, 1996; Atwell et al., J Mol Biol 270:26-35, 1997; and U.S. Pat. No. 7,695,936).

Linker peptides can be included between heavy and light variable regions of an scFv or between an scFv and a constant region. Linkers are short peptide conferring flexibility often predominantly occupied by Gly, Ala and/or Ser. Some exemplary linkers are Gly-Gly-Ala-Ala (SEQ ID NO:82), Gly-Gly-Gly-Gly-Ser (SEQ ID NO:83), Leu-Ala-Ala-Ala-Ala (SEQ ID NO:84) and multimers thereof.

Activation of T cells is demonstrated in the application by (i) increase of the expression of IFNγ, IL-2 and granzyme B, and (ii) cytotoxicity against target cells. In the absence of target antigen-expressing cells, these trispecific antibodies do not activate T cells above background of negative controls, thus do not show non-specific activation of T cells.

### V. Selection of Constant Region

Many of the formats for a trispecific antibody include at least a portion of a human constant region. The exemplified format includes first and second heavy chains including first and second constant regions. The first and second constant regions typically include CH1, hinge, CH2 and CH3 regions with the C-terminal lysine of the CH3 region typically being omitted. The first or second constant regions can be the same as one another, or can differ from one another, as result for example of including knobs in one heavy chain constant region and holes in the other. If the heavy chain constant regions are different from each other they are typically still of the same isotype and subtype.

The choice of constant region depends, in part, whether antibody-dependent cell-mediated cytotoxicity, antibody dependent cellular phagocytosis and/or complement dependent cytotoxicity are desired. For example, human isotypes IgG1 and IgG3 have complement-dependent cytotoxicity and human isotypes IgG2 and IgG4 do not. Light chain constant regions can be lambda or kappa. Human IgG1 and IgG3 also induce stronger cell mediated effector functions than human IgG2 and IgG4. Here although ADCC, ADCP and CDC may be useful in providing an additional mechanism of action against cancer or infected cells bound by one binding site of the trispecific antibodies, it is not useful for agonizing costimulatory molecules by the other binding sites to activate immune cells.

One or several amino acids at the amino or carboxy terminus of the light and/or heavy chain, such as the C-terminal lysine of the heavy chain, may be missing or derivatized in a proportion or all of the molecules. Thus, for example, a preferred linkage site between a heavy chain constant region and scFv is the penultimate glycine residue of the heavy chain constant region rather than the terminal lysine. Amino acid substitutions can be made in the constant regions to reduce or increase effector functions such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004). For example, there are many known mutations in IgG Fc that increase FcRn binding. Exemplary substitutions include Gln at position 250 and/or Leu at position 428, Ser or Asn at position 434, Tyr at position 252, Thr at position 254, Glu at position 256, and Ala at position 434 (EU numbering). Increased FcRn binding is advantageous in making the hybrid proteins of the present invention compete more strongly with endogenous IgG for binding to FcRn. Also numerous mutations are known for reducing any of ADCC, ADCP or CDC. (see, e.g., Winter et al., U.S. Pat. No. 5,624,821; Tso et al., U.S. Pat. No. 5,834,597; and Lazar et al., Proc. Natl. Acad. Sci. USA 103:4005, 2006). For example, substitution of any of amino acid residues at positions 234, 235, 236 and/or 237 reduce affinity for Fcγ receptors, particularly FcγRI receptor (see, e.g., U.S. Pat. No. 6,624,821). Optionally, amino acid residues at positions 234, 236 and/or 237 in human IgG2 are substituted with Ala and at position 235 with Gln or Glu (See, e.g., U.S. Pat. No. 5,624,821). Other substitutions reducing effector functions include Ala at position 268, Gly or Ala at position 297, Leu at position 309, Ala at position 322, Gly at position 327, Ser at position 330, Ser at position 331, Ser at position 238, Ala at position 268, Leu at position 309. Alanine substitutions at positions 234 and 235 by Eu numbering are preferred.

Human constant regions show allotypic variation and isoallotypic variation between different individuals, that is, the constant regions can differ in different individuals at one or more polymorphic positions. Isoallotypes differ from allotypes in that sera recognizing an isoallotype bind to a non-polymorphic region of one or more other isotypes.

### VI. Expression of Recombinant Antibodies

Antibodies are typically produced by recombinant expression. A standard antibody requires expression of heavy and light chains. The exemplified format for trispecific antibodies requires expression for three antibody chains, two heavy chains and one line chain. Multiple chains can be expressed from the same or different vectors. Recombinant polynucleotide constructs typically include an expression control sequence operably linked to the coding sequences of antibody chains, including naturally associated or heterologous expression control elements, such as a promoter. The expression control sequences can be promoter systems in vectors capable of transforming or transfecting eukaryotic or prokaryotic host cells. Once the vector(s) has/have been incorporated into the appropriate host, the host is maintained under conditions suitable for high level expression of the nucleotide sequences and the collection and purification of antibodies.

Expression vectors are typically replicable in the host organisms either as episomes or as an integral part of the host chromosomal DNA. Commonly, expression vectors contain selection markers, e.g., ampicillin resistance or hygromycin resistance, to permit detection of those cells transformed with the desired DNA sequences.

E. coli is one prokaryotic host useful for expressing antibodies, particularly antibody fragments. Microbes, such as yeast, are also useful for expression. Saccharomyces is a yeast host with suitable vectors having expression control sequences, an origin of replication, termination sequences, and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

Mammalian cells can be used for expressing nucleotide segments encoding immunoglobulins or fragments thereof. See Winnacker, From Genes to Clones, (VCH Publishers, NY, 1987). A number of suitable host cell lines capable of secreting intact heterologous proteins have been developed, and include CHO cell lines, various COS cell lines, HeLa cells, HEK293 cells, L cells, and non-antibody-producing myelomas including Sp2/0 and NSO. The cells can be nonhuman. Expression vectors for these cells can include expression control sequences, such as an origin of replication, a promoter, an enhancer (Queen et al., Immunol. Rev. 89:49 (1986)), and necessary processing information sites, such as ribosome binding sites, RNA splice sites, polyadenylation sites, and transcriptional terminator sequences. Expression control sequences can include promoters derived from endogenous genes, cytomegalovirus, SV40, adenovirus, bovine papillomavirus, and the like. See Co et al., J. Immunol. 148:1149 (1992).

Alternatively, antibody coding sequences can be incorporated in transgenes for introduction into the genome of a transgenic animal and subsequent expression in the milk of the transgenic animal (see, e.g., U.S. Pat. Nos. 5,741,957; 5,304,489; and 5,849,992). Suitable transgenes include coding sequences for light and/or heavy chains operably linked with a promoter and enhancer from a mammary gland specific gene, such as casein or beta lactoglobulin.

The vectors containing the DNA segments of interest can be transferred into the host cell by methods depending on the type of cellular host. For example, calcium chloride transfection is commonly utilized for prokaryotic cells, whereas calcium phosphate treatment, electroporation, lipofection, biolistics, or viral-based transfection can be used for other cellular hosts. Other methods used to transform mammalian cells include the use of polybrene, protoplast fusion, liposomes, electroporation, and microinjection. For production of transgenic animals, transgenes can be microinjected into fertilized oocytes or can be incorporated into the genome of embryonic stem cells, and the nuclei of such cells transferred into enucleated oocytes.

Having introduced vector(s) encoding antibody heavy and light chains into cell culture, cell pools can be screened for productivity and quality of antibodies in serum-free media. Top-producing cell pools can then be subjected to FACS-based single-cell cloning to generate monoclonal lines. Antibodies produced by single cell clones can also be tested for turbidity, filtration properties, PAGE, IEF, UV scan, HP-SEC, carbohydrate-oligosaccharide mapping, mass spectrometry, and binding assay, such as ELISA or Biacore. A selected clone can then be banked in multiple vials and stored frozen for subsequent use.

Once expressed, desired antibodies can be purified from other antibody forms as well as cellular debris and other impurities according to standard procedures of the art, including protein A capture, HPLC purification, column chromatography, gel electrophoresis and the like (see generally, Scopes, Protein Purification (Springer-Verlag, NY, 1982)). Purification of trispecific antibodies can include separation from bispecific antibodies resulting from homodimerization of heavy chains and other mispairing.

Methodology for commercial production of antibodies can be employed, including codon optimization, selection of promoters, selection of transcription elements, selection of terminators, serum-free single cell cloning, cell banking, use of selection markers for amplification of copy number, CHO terminator, or improvement of protein titers (see, e.g., U.S. Pat. Nos. 5,786,464; 6,114,148; 6,063,598; 7,569,339; W02004/050884; W02008/012142; W02008/012142; W02005/019442; W02008/107388; W02009/027471; and U.S. Pat. No. 5,888,809).

### VII. Nucleic Acids

The invention further provides nucleic acids encoding heavy and light chains of antibodies described above. For a trispecific antibody including first and second heavy chains and a light chain, two nucleic acids encode the first and second heavy chains and a third the light chain. Optionally, such nucleic acids further encode a signal peptide and can be expressed with the signal peptide linked to the free N-terminus of the variable region coding sequences of the nucleic acids. Nucleic acids encoding antibody chains can be operably linked with regulatory sequences, such as a promoter, enhancer, ribosome binding site, transcription termination signal, and the like, to ensure expression of the coding sequences. The nucleic acids encoding heavy and light chains can occur in isolated form or can be cloned into one or more vectors. The nucleic acids can be synthesized by, for example, solid state synthesis or PCR of overlapping oligonucleotides. Nucleic acids encoding heavy chain(s) and a light chain(s) can be joined as one contiguous nucleic acid, e.g., within an expression vector, or can be separate, e.g., each cloned into its own expression vector.

### VIII. Methods of Treatment and Pharmaceutical Compositions

The trispecific antibodies of the invention can be used for treating cancers including any of those in which one binding site of the trispecific antibody binds to a target expressed or overexpressed in the cancer, such as those disclosed above. The trispecific antibodies can be used to treat solid tumors, and hematological malignancies. Hematological malignancies include leukemia (e.g., T cell large granular lymphocyte leukemia), lymphoma (Hodgkin's or Non-Hodgkin's), or multiple myeloma. Solid tumors include skin (e.g., melanoma), ovarian, endometrial, kidney, liver, pancreas, bladder, breast, ovarian, prostate, rectum, colon, gastric, intestinal, pancreatic, lung, thymus, thyroid, kidney and brain.

Trispecific antibodies of the invention can also be used for treating infections by infectious organisms when the trispecific antibody has one binding site specifically binding to an antigen expressed on a pathogen or in infected cells but not in matched uninfected cells. Such an antigen can be encoded by the infectious organism or can be expressed by the cell in response to infection by the infectious organism. Examples of such antigens expressed in infected cells are human immune deficiency virus (HIV) glycoproteins gp41 and gp120, human T-cell leukemia virus type 1 (HTLV-1) Env protein, herpes simplex virus (HSV) glycoproteins gB and gH, influenza hemagglutinin (HA) and neuraminidase (NA), and respiratory syncytial virus (RSV) F protein. Examples of pathogenic infections treatable with trispecific antibodies include viral, bacterial, protozoan or fungal infection. Some example of viral infections include HIV, hepatitis (A, B, or C), herpes virus (e.g., VZV, HSV-1, HAV-6, HSV-II, CMV, and Epstein Barr virus), adenovirus, XMRV, influenza virus, flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, MLV-related virus, papillomavirus, molluscum virus, poliovirus, rabies virus, JC virus and arboviral encephalitis virus. Some examples of bacterial infections include Chlamydia, rickettsial bacteria, mycobacteria, staphylococci, streptococci, pneumonococci, meningococci and conococci, Klebsiella, Proteus, Serratia, Pseudomonas, Legionella, diphtheria, Salmonella, bacilli, cholera, tetanus, botulism, anthrax, plague, leptospirosis, Lymes disease bacteria, streptococci, or Neisseria. Some examples of pathogenic fungi include Candida, Aspergillus, Cryptococcus, Histoplasma, Pneumocystis and Stachybotrys. Examples of protozoa include Cryptosporidium, Giardia lamblia and plasmodium.

Trispecific antibodies of the invention in which the first binding site is directed against an antigen on an autoreactive cell can be used to eliminate autoreactive cells in treatment of autoimmune disease. In autoimmune diseases, the body develops a cellular and/or humoral immune response against one of its own antigens leading to destruction of that antigen, and potentially crippling and/or fatal consequences.

Exemplary autoimmune disease include type 1 diabetes, Crohn's disease, ulcerative colitis, multiple sclerosis, stiff person syndrome, rheumatoid arthritis, myasthenia gravis, lupus crythematosus, celiac disease, psoriasis, uveitis, alopecia areata, and primary biliary cirrhosis, asthma, allergies, vitiligo, polymyositis, allergic dermatitis, and Behçet's disease, idiopathic thrombocytopenia purpura, Kawasaki disease, Guillain-Barre syndrome, chronic inflammatory demyelinating systemic lupus erythematosus, autoimmune neutropenia type 1 diabetes, acute disseminated encephalomyelitis, acute motor axonal neuropathy, Addison's disease, adiposis dolorosa, adult-onset Still's disease, ankylosing spondylitis, anti-glomerular basement membrane nephritis, anti-neutrophil cytoplasmic antibody-associated vasculitis, anti-N-methyl-D-aspartate receptor encephalitis, antiphospholipid syndrome, antisynthetase syndrome, aplastic anemia, autoimmune angioedema, autoimmune encephalitis, autoimmune enteropathy, autoimmune hemolytic anemia, autoimmune hepatitis, autoimmune inner car disease, autoimmune lymphoproliferative syndrome, autoimmune oophoritis, autoimmune orchitis, autoimmune pancreatitis, autoimmune polyendocrine syndrome, autoimmune polyendocrine syndrome type 2, autoimmune polyendocrine syndrome type 3, autoimmune progesterone dermatitis, autoimmune retinopathy, autoimmune thrombocytopenia purpura, autoimmune thyroiditis, autoimmune urticaria, autoimmune uveitis, Balo concentric sclerosis, Behçet's disease, Bickerstaff's encephalitis, bullous pemphigoid, chronic fatigue syndrome, Churg-Strauss syndrome, cicatricial pemphigoid, Cogan syndrome, cold agglutinin disease, complex regional pain syndrome, CREST syndrome, dermatitis herpetiformis, dermatomyositis, discoid lupus erythematosus, endometriosis, enthesitis, enthesitis-related arthritis, cosinophilic esophagitis, cosinophilic fasciitis, epidermolysis bullosa acquisita, crythema nodosum, essential mixed cryoglobulinemia, Evans syndrome, Felty syndrome, fibromyalgia, gastritis, gestational pemphigoid, giant cell arteritis, Goodpasture syndrome, Graves' disease, Graves ophthalmopathy, Hashimoto's encephalopathy, Hashimoto thyroiditis, Henoch-Schonlein purpura, hidradenitis suppurativa, idiopathic dilated cardiomyopathy, idiopathic inflammatory demyelinating diseases, IgA nephropathy, IgG4-related systemic disease, inclusion body myositis, inflammatory bowel disease (IBD), intermediate uveitis, interstitial cystitis, juvenile arthritis, Kawasaki's disease, Lambert-Eaton myasthenic syndrome, leukocytoclastic vasculitis, lichen planus, lichen sclerosus, ligneous conjunctivitis, linear IgA disease, lupus nephritis, lupus vasculitis, Lyme disease, Ménière's disease, microscopic colitis, microscopic polyangiitis, mixed connective tissue disease, Mooren's ulcer, morphea, Mucha-Habermann disease, myocarditis, myositis, neuromyelitis optica, neuromyotonia, opsoclonus myoclonus syndrome, optic neuritis, Ord's thyroiditis, palindromic rheumatism, paraneoplastic cerebellar degeneration, Parry Romberg syndrome, Parsonage-Turner syndrome, pediatric autoimmune neuropsychiatric disorder associated with Streptococcus, pemphigus vulgaris, pernicious anemia, pityriasis lichenoides et varioliformis acuta, POEMS syndrome, polyarteritis nodosa, polymyalgia rheumatica, polymyositis, postmyocardial infarction syndrome, postpericardiotomy syndrome, primary biliary cirrhosis, primary immunodeficiency, primary sclerosing cholangitis, progressive inflammatory neuropathy, psoriatic arthritis, pure red cell aplasia, pyoderma gangrenosum, Raynaud's phenomenon, reactive arthritis, relapsing polychondritis, restless leg syndrome, retroperitoneal fibrosis, rheumatic fever, rheumatoid vasculitis, sarcoidosis, Schnitzler syndrome, scleroderma, Sjogren's syndrome, subacute bacterial endocarditis, Susac's syndrome, Sydenham chorea, sympathetic ophthalmia, systemic scleroderma, thrombocytopenia, Tolosa-Hunt syndrome, transverse myelitis, undifferentiated connective tissue disease, urticaria, urticarial vasculitis, and vasculitis.

Trispecific antibodies are administered in an effective regime meaning a dosage, route of administration and frequency of administration that delays the onset, reduces the severity, inhibits further deterioration, and/or ameliorates at least one sign or symptom of a condition. If a subject is already suffering from a disorder, the regime can be referred to as a therapeutically effective regime. If the subject is at elevated risk of the condition relative to the general population but is not yet experiencing symptoms, the regime can be referred to as a prophylactically effective regime. In some instances, therapeutic or prophylactic efficacy can be observed in an individual subject relative to historical controls or past experience in the same subject. In other instances, therapeutic or prophylactic efficacy can be demonstrated in a preclinical or clinical trial in a population of treated subjects relative to a control population of untreated subjects.

Preferably a trispecific antibody exhibits at least additive and more preferably synergistic activity against a cancer, infectious organism, infected cell or autoreactive cell compared with its component antibodies individually (i.e., an antibody specifically binding to a cell bearing a target antigen in a standard antibody format and scFvs specifically binding to CD3 and CD28). Synergy is preferably assessed quantitatively such as discussed by Tallarida, Genes Cancer. 2011 November; 2(11): 1003-1008. Preferably a trispecific antibody also exhibits increased activity compared with a mixture of its component antibodies, each at equimolar concentration with the trispecific antibody. Such activity can be measured, for example, as cytotoxicity or cytostaticity against cancer cells or infected cells or autoreactive cells expressing an antigen specifically bound by one binding site of the trispecific antibody in the presence of immune cells expressing CD3 and CD28 bound by the other binding sites of the trispecific antibody.

Exemplary dosages for a trispecific antibody are 0.01-20, or 0.5-5, or 0.01-1, or 0.01-0.5 or 0.05-0.5 mg/kg body weight (e.g., 0.1, 0.5, 1, 2, 3, 4 or 5 mg/kg) or 10-1500 mg as a fixed dosage. The dosage depends on the condition of the patient and response to prior treatment, if any, whether the treatment is prophylactic or therapeutic and whether the disorder is acute or chronic, among other factors.

Administration can be parenteral, intravenous, oral, subcutaneous, intra-arterial, intracranial, intrathecal, intraperitoneal, topical, intranasal or intramuscular. Administration into the systemic circulation by intravenous or subcutaneous administration is preferred. Intravenous administration can be, for example, by infusion over a period such as 30-90 min.

The frequency of administration depends on the half-life of the trispecific antibody in the circulation, the condition of the subject and the route of administration among other factors. The frequency can be daily, weekly, monthly, quarterly, or at irregular intervals in response to changes in the patient's condition or progression of the disorder being treated. An exemplary frequency for intravenous administration is between weekly and quarterly over a continuous cause of treatment, although more or less frequent dosing is also possible. For subcutaneous administration, an exemplary dosing frequency is daily to monthly, although more or less frequent dosing is also possible.

The number of dosages administered depends on whether the disorder is acute or chronic and the response of the disorder to the treatment. For acute disorders or acute exacerbations of chronic disorders, between 1 and 10 doses are often sufficient. Sometimes a single bolus dose, optionally in divided form, is sufficient for an acute disorder or acute exacerbation of a chronic disorder. Treatment can be repeated for recurrence of an acute disorder or acute exacerbation. For chronic disorders, a bispecific antibody can be administered at regular intervals, e.g., weekly, fortnightly, monthly, quarterly, every six months for at least 1, 5 or 10 years, or the life of the subject.

Pharmaceutical compositions are preferably suitable for parenteral administration to a human (e.g., according to the standard of the FDA). Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more pharmaceutically acceptable carriers, diluents, excipients or auxiliaries. Pharmaceutically acceptable means suitable for human administration, e.g., approved or approvable by the FDA. The formulation depends on the route of administration chosen. For injection, antibodies can be formulated in aqueous solutions, preferably in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively antibodies can be in lyophilized form for constitution with a suitable vehicle, e.g., sterile pyrogen-free water, before use.

Treatment with the trispecific antibodies of the invention can be combined with other treatments effective against the disorder being treated. When used in treating cancer, the trispecific antibodies of the invention can be combined with chemotherapy, radiation, stem cell treatment, surgery or treatment with other biologics such as Herceptin™ (trastuzumab) against the HER2 antigen, Avastin™ (bevacizumab) against VEGF, antibodies to the EGF receptor, such as Erbitux™ (cetuximab) and Vectibix™ (panitumumab), antibodies to PD-1, such as Keytruda (pembrolizumab), Opdivo (nivolumab), Libtayo (cemiplimab-rwlc), Jemperli (dostarlimab-gxly), Zynyz (retifanlimab-dlwr) and Loqtorzi (toripalimab-tpzi), or antibodies to PD-L1, such as Tecentriq (atczolizumab), Bavencio (avelumab) and Imfinzi (durvalumab). Chemotherapy agents include chlorambucil, cyclophosphamide or melphalan, carboplatinum, daunorubicin, doxorubicin, idarubicin, and mitoxantrone, methotrexate, fludarabine, and cytarabine, etoposide or topotecan, vincristine and vinblastine. Chemokines and cytokines, interferon type 1 or IL-12, to deliver Signal 3 can also be used for co-treatment. For infections, treatment can be in combination with antibiotics, anti-virals, anti-fungal or anti-protozoan agents or the like.

Monospecific antibodies specifically binding to CD28 can be used in construction of bispecific or trispecific antibodies as described herein.

### IX. Other Methods

The antibodies of the invention also find use in diagnostic, prognostic and laboratory methods. They may be used to measure the level of an antigen expressed by a cancer or in the circulation of a patient with a cancer, to determine if the level is measurable or even elevated, and therefore to follow and guide treatment of the cancer, because cancers associated with measurable or elevated levels of an antigen are most susceptible to treatment with a trispecific antibody comprising a binding site binding to a cancer-associated antigen. They can likewise be used to determined levels of CD28 or CD3 on effector cells. The antibodies can be used for an ELISA assay, radioimmunoassay or immunohistochemistry among others. The antibodies can be labeled with fluorescent molecules, spin-labeled molecules, enzymes or radioisotopes, and may be provided in the form of a kit with all the necessary reagents to perform the assay.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### EXAMPLES

### Example 1: Experimental Procedures, Methods, and Materials

Gene cloning, mutagenesis, plasmid construction, expression and purification of proteins, cell culturing, ELISA, flow cytometry, and hybridoma generation were carried out following standard laboratory techniques such as those described by Green and Sambrook (Molecular Cloning, A Laboratory Manual, 4th ed., 2012, Cold Spring Harbor Laboratory Press, Cold Spring Harbor, NY), Greenfield (Antibodies, A Laboratory Manual, 2nd ed., 2014, Cold Spring Harbor Laboratory Press, Cold Spring Harbor, NY), Kostelny et al. (Int. J. Cancer 93:556-565, 2001), Cole et al. (J. Immunol. 159:3613-3621, 1997) and Tsurushita et al. (Methods 36:69-83, 2005), and in vendors' protocols.

Stable transfection into a Chinese hamster ovary cell line CHO-K1 was carried out by electroporation. Before transfection, an expression vector was linearized using an appropriate restriction enzyme. In a typical experiment, approximately 107 CHO-K1 cells were transfected with 20 μg of linearized plasmid, suspended in SFM4CHO media (Cytiva, Marlborough, MA), and plated at 100 μl/well in several 96-well plates after appropriate dilutions of cells. After 24 to 48 hr, SFM4CHO media containing 20 μg/ml of puromycin was added at 100 μl/well for isolation of stable transfectants. Approximately ten days after the initiation of selection, culture supernatants of CHO-K1 transfectants were assayed for antibody production by ELISA as described below.

Expression level of chimeric, humanized, bispecific, and trispecific IgG/kappa antibodies in culture supernatants was measured by sandwich ELISA. In typical experiments, an ELISA plate was coated with goat anti-human IgG Fc-specific polyclonal antibody (Jackson ImmunoResearch, West Grove, PA) in PBS, washed with Wash Buffer (PBS containing 0.05% Tween 20), blocked with ELISA Buffer (PBS containing 2% skim milk and 0.05% Tween 20), and washed with Wash Buffer. Test samples, such as culture supernatants and purified antibodies, appropriately diluted in ELISA Buffer were then applied to the plate. An appropriate humanized, bispecific, or trispecific IgG/kappa antibody was used as a standard. After incubating the plate for 60 min at room temperature (or overnight at 4° C.) and washing with Wash Buffer, bound antibodies were detected using HRP-conjugated goat anti-human kappa chain polyclonal antibody (Bethyl Laboratories, Montgomery, TX). After incubating the plate for 30 min at room temperature and washing with Wash Buffer, color development was initiated by adding ABTS substrate (Sigma-Aldrich, St. Louis, MO) and stopped with 2% oxalic acid. Absorbance was read at 405 nm.

CHO-K1 stable transfectants highly producing a recombinant antibody, such as chimeric, humanized, bispecific, and trispecific IgG/kappa antibodies of this invention, were expanded in SFM4CHO until the cell viability became less than 50%. After centrifugation and filtration, culture supernatants were loaded onto a protein A column (HiTrap™ MABSelect™ SuRe, Cytiva). The column was washed with PBS before the antibody was eluted with 0.1 M glycine-HCl buffer (pH 3.0) containing 0.1M NaCl or 0.1M sodium acetate buffer (pH 3.6). The buffer of eluted antibodies was changed to PBS by dialysis. Antibody concentration was determined by measuring absorbance at 280 nm (1 mg/ml=1.4 OD).

Human peripheral blood mononuclear cells (PBMC) were isolated by density gradient centrifugation from the buffy coat obtained from Stanford Blood Center (Palo Alto, CA).

For the location of amino acid residues in chimeric, humanized, and human IgG1/kappa antibodies, the numbering system of Kabat et al. (Sequences of Proteins of Immunological Interest, National Institutes of Health, Bethesda, MD, 1987 and 1991), which is known as Kabat numbering for variable regions and Eu numbering for constant regions, was used.

### Example 2: Generation of a Humanized Anti-CD28 Monoclonal Antibody

CD28 is a costimulatory molecule that triggers Signal 2 of T cell activation when associated with Signal 1 that is triggered by the interaction of TCR with its cognate peptide-MHC (or HLA) complex. The amino acid sequence of the extracellular region of human CD28 (SEQ ID NO:1) is the same as that of cynomolgus monkeys (Macaca fascicularis) and rhesus monkeys (Macaca mulatta).

Mouse hybridomas producing monoclonal antibodies against CD28 were generated at JN Biosciences (Mountain View, CA) following standard cell fusion techniques using GenomONE CF EX Cell Fusion Reagent (Cosmo Bio, Carlsbad, CA). For mouse immunization, the extracellular region of human CD28 fused to human Fcγ1 region (CD28-Fc; Cat. No. 11524-H02H, Sino Biological, Wayne, PA) was used. Mouse monoclonal IgG antibodies secreted in culture supernatants of hybridoma cells were subjected to a series of screening to identify the antibodies that specifically bind to CD28-Fc and human T cells. One of such anti-CD28 monoclonal IgG/kappa antibodies was named TEC1.

The heavy and light chain variable regions (VH and VL, respectively) of TEC1 was determined by cDNA sequencing as described in Tsurushita et al. (supra). The amino acid sequence of TEC1 VH is MAVLALLFCLVTFPSCILSQVQLKESGPGLVAPSQSLSITCTVSGFSLTGYGIYWVRQPPG KGLEWLGMIWGDGSTDYNSALRSRLSINKDNSKSQVFLKMNSLQTDDTARYYCARDR GYYGSSSLYYYAMDSWGQGTSVTVSS (SEQ ID NO:2). Mature TEC1 VH starts at a glutamate residue at position 20 of SEQ ID NO:2. The amino acid sequences of CDRH1, CDRH2 and CDRH3 of TEC1 VH are GYGIY (SEQ ID NO:3), MIWGDGSTDYNSALRS (SEQ ID NO:4), and DRGYYGSSSLYYYAMDS (SEQ ID NO:5), respectively.

The amino acid sequence of TEC1 VL is MESDTLLLWVLLLWVPGSTGDIVLTQSPASLAVSLGQRATISCRASESVEYYGTSLMQW YQQKPGQPPKLLIYAASNVESGVPARFSGSGSGTDFSLNIHPVEEDDIAMYFCQQSRKVP WTFGGGTKLEIK (SEQ ID NO:6). Mature TEC1 VL starts at an aspartate residue at position 21 of SEQ ID NO:6. The amino acid sequences of CDRL1, CDRL2 and CDRL3 of TEC1 VL are RASESVEYYGTSLMQ (SEQ ID NO:7), AASNVES (SEQ ID NO:8), and QQSRKVPWT (SEQ ID NO:9), respectively.

Humanization of the VH and VL regions of mouse anti-CD28 antibody TEC1 was carried out following the general procedure described by Tsurushita et al. (supra). A three-dimensional molecular model of the TEC1 variable regions was first constructed using an appropriate software. The framework amino acid residues important for the formation of the CDR (complementarity-determining region) structure or necessary for antigen binding were then identified using the molecular model. In parallel, cDNA-derived human VH and VL amino acid sequences with high homology to the TEC1 VH and VL amino acid sequences, respectively, were selected. Lastly, CDR sequences together with framework amino acid residues identified to be important for the formation of the antigen-binding site were grafted from the TEC1 variable regions into the corresponding selected human framework sequences.

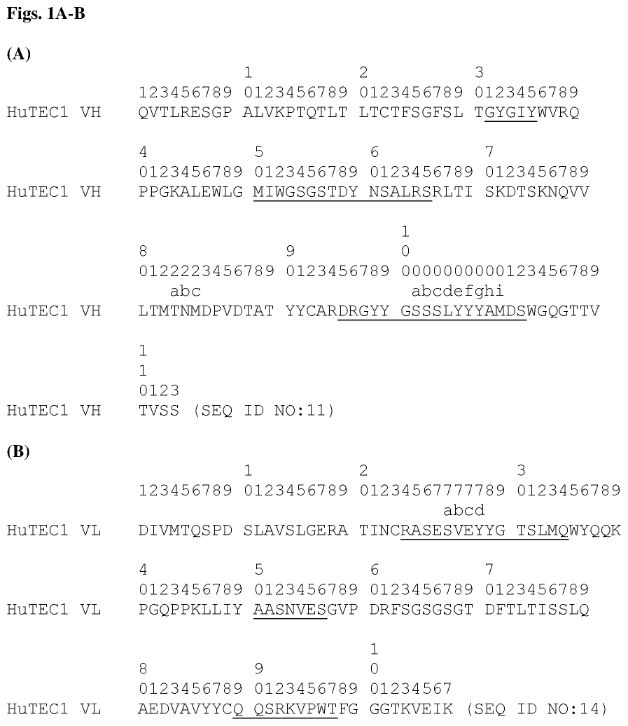

The amino acid sequence of the resultant humanized TEC1 (HuTEC1) VH is MAVLALLFCLVTFPSCILSQVTLRESGPALVKPTQTLTLTCTFSGFSLTGYGIYWVRQPPG KALEWLGMIWGSGSTDYNSALRSRLTISKDTSKNQVVLTMTNMDPVDTATYYCARDR GYYGSSSLYYYAMDSWGQGTTVTVSS (SEQ ID NO:10). Mature HuTEC1 VH starts at a glutamate residue at position 20 of SEQ ID NO:10. FIG. 1A shows the amino acid sequence of mature HuTEC1 VH (SEQ ID NO:11) to indicate the position of each amino acid residue and the locations of the complementarity determining regions (CDRs) based on the definition by Kabat et al. (supra). The amino acid sequences of CDRH1, CDRH2 and CDRH3 of HuTEC1 VH are GYGIY (SEQ ID NO:3), MIWGSGSTDYNSALRS (SEQ ID NO:12), and DRGYYGSSSLYYYAMDS (SEQ ID NO:5), respectively. During humanization, a serine residue was placed at the fifth position in CDRH2 of HuTEC1 VH (position 54 in FIG. 1A) where an aspartate residue exists in the parental TEC1 VH.

The amino acid sequence of the resultant HuTEC1 VL is MESDTLLLWVLLLWVPGSTGDIVMTQSPDSLAVSLGERATINCRASESVEYYGTSLMQ WYQQKPGQPPKLLIYAASNVESGVPDRFSGSGSGTDFTLTISSLQAEDVAVYYCQQSRK VPWTFGGGTKVEIK (SEQ ID NO:13). Mature HuTEC1 VL starts at an aspartate residue at position 21 of SEQ ID NO:13. FIG. 1B shows the amino acid sequence of mature HuTEC1 VL (SEQ ID NO:14) to indicate the position of each amino acid residue and the locations of the CDRs based on the definition by Kabat et al. (supra). The amino acid sequences of CDRL1,CDRL2 and CDRL3 of HuTEC1 VL are RASESVEYYGTSLMQ (SEQ ID NO:7), AASNVES (SEQ ID NO:8), and QQSRKVPWT (SEQ ID NO:9), respectively.

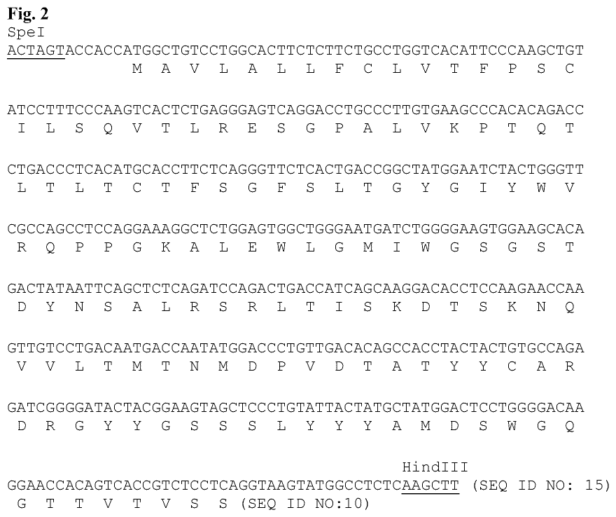

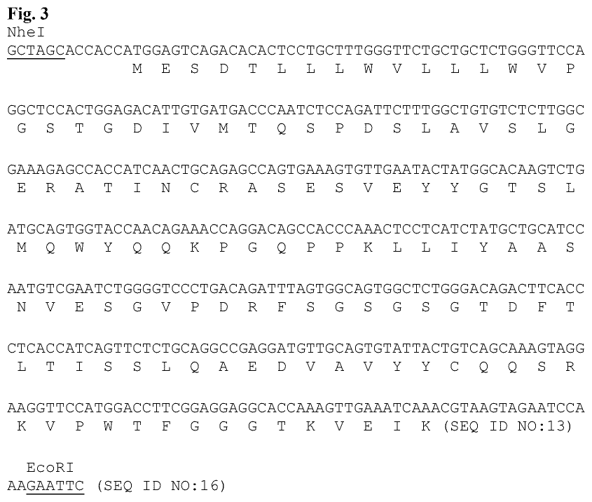

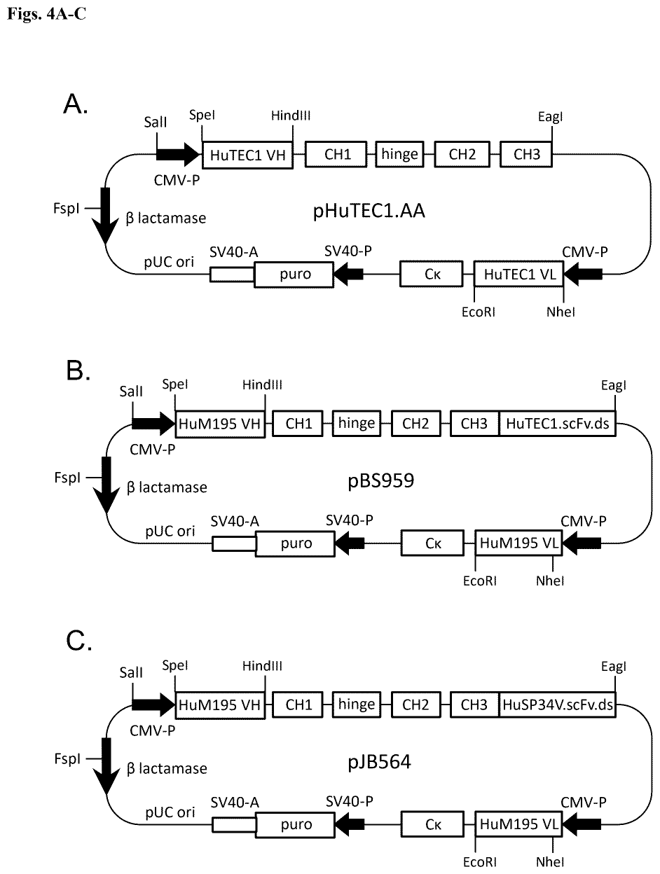

A gene encoding HuTEC1 VH (SEQ ID NO:15) was synthesized as an exon including a signal peptide, a splice donor signal, an SpeI site at the 5′ end, and a HindIII site at the 3′ end (FIG. 2). A gene encoding HuTEC1 VL (SEQ ID NO:16) was likewise synthesized as an exon including a signal peptide, a splice donor signal, an NheI site at the 5′ end, and an EcoRI site at the 3′ end (FIG. 3). The HuTEC1 VH and VL genes were cloned between the Spe1 and HindIII sites (for VH) or between the NheI and EcoRI sites (for VL) of a mammalian expression vector for production of humanized IgG1/kappa antibody. The schematic structure of the resultant expression vector named pHuTEC1.AA is shown in FIG. 4A. Humanized anti-CD28 IgG1/kappa antibody (HuTEC1.AA) is produced from pHuTEC1.AA in mammalian cells.

Proceeding clockwise from the Sall site in FIG. 4A, pHuTEC1.AA contains the heavy chain transcription unit starting with the human cytomegalovirus (CMV) major immediate early promoter and enhancer (CMV-P) to initiate transcription of the antibody heavy chain gene. The CMV promoter is followed by the VH exon encoding HuTEC1 VH, a genomic sequence containing the human gamma-1 heavy chain constant region including the CH1, hinge, CH2 and CH3 exons with the intervening introns, and the polyadenylation site following the CH3 exon. The CH2 region carries amino acid substitutions from leucine to alanine at positions 234 and 235 (Eu numbering; Kabat et al., supra) to eliminate effector functions (Hazareh et al., J. Virol. 75:12161-12168, 2001). After the heavy chain gene sequence, the light chain transcription unit begins with the CMV promoter, followed by the exon encoding HuTEC1 VL and a genomic sequence containing the human kappa chain constant region (C_{κ}) with part of the intron preceding it, and the polyadenylation site following the C_{κ} exon. The light chain gene is then followed by the SV40 early promoter (SV40-P), the puromycin N-acetyl-transferase gene (puro) for resistance to puromycin, and a segment containing the SV40 polyadenylation site (SV40-A). Finally, the plasmid contains a part of the plasmid pUC19, comprising the bacterial origin of replication (pUC ori) and beta-lactamase gene (β lactamase). Locations of relevant restriction enzyme sites are shown in FIG. 4A. Arrows indicate orientation of transcription.

Amino acid sequences of the CH1, hinge, CH2, and CH3 regions of human gamma-1 heavy chain encoded in pHuTEC1.AA are ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKV (SEQ ID NO:17), EPKSCDKTHTCPPCP (SEQ ID NO:18), APEAAGGPSVFLFPPKPKDTLMISRTPEVTCVVVDVSHEDPEVKFNWYVDGVEVHNAK TKPREEQYNSTYRVVSVLTVLHQDWLNGKEYKCKVSNKALPAPIEKTISKAK (SEQ ID NO:19), and

GQPREPQVYTLPPSRDELTKNQVSLTCLVKGFYPSDIAVEWESNGQPENNYKTTPPVLD SDGSFFLYSKLTVDKSRWQQGNVFSCSVMHEALHNHYTQKSLSLSPGK (SEQ ID NO:20), respectively.

Amino acid sequence of the human kappa constant region (C_{κ}) encoded in pHuTEC1.AA is

(SEQ ID NO: 21)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC.

Amino acid sequence of a mature heavy chain encoded in pHuTEC1.AA is

(SEQ ID NO: 22)
QVTLRESGPALVKPTQTLTLTCTFSGFSLTGYGIYWVRQPPGKALEWLGMIWGSGSTDY
NSALRSRLTISKDTSKNQVVLTMTNMDPVDTATYYCARDRGYYGSSSLYYYAMDSWG
QGTTVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHTCP
PCPAPEAAGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGK.

Amino acid sequence of a mature light chain encoded in pHuTEC1.AA is

(SEQ ID NO: 23)
DIVMTQSPDSLAVSLGERATINCRASESVEYYGTSLMQWYQQKPGQPPKLLIYAASNVE
SGVPDRFSGSGSGTDFTLTISSLQAEDVAVYYCQQSRKVPWTFGGGTKVEIKRTVAAPS
VFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC.

Stable transfection of CHO-K1 cells with pHuTEC1.AA, isolation and expansion of CHO-K1 transfectants producing HuTEC1.AA, and purification of HuTEC1.AA by protein A column chromatography were carried out as described above. SDS-PAGE analysis of purified HuTEC1.AA under reducing conditions showed only two predominant bands of the expected size (roughly 50 kDa heavy chain and 25 kDa light chain).

HuTEC1.AA maintained the affinity and specificity of TEC1. Binding of HuTEC1.AA to CD28 was examined by ELISA. An ELISA plate was coated with 1 μg/ml of CD28-Fc overnight at 4° C., washed with Wash Buffer, blocked with ELISA Buffer, and washed with Wash Buffer. HuTEC1.AA appropriately diluted in ELISA Buffer was then applied to the plate. After incubating the plate for 60 min at room temperature, and washing with Wash Buffer, HuTEC1.AA binding to CD28-Fc was detected using HRP-conjugated goat anti-human kappa chain polyclonal antibody. After incubating the plate for 30 min at room temperature and washing with Wash Buffer, color development was initiated with ABTS substrate and stopped with 2% oxalic acid. Absorbance was read at 405 nm. HuTEC1.AA bound to CD28 in a dose-dependent manner. The EC₅₀ value of HuTEC1.AA for binding to CD28 was 291 ng/ml.

### Example 3: Construction of a Bispecific Antibody That Binds to CD33 and CD28.

An expression vector for a bispecific antibody that binds to CD33 (also called Siglec-3) and CD28, which was named pBS959 (FIG. 4B), has the same structure as pHuTEC1.AA except that (i) the VH and VL genes of pHuTEC1.AA are substituted respectively with the HuM195 VH and VL genes derived from a humanized anti-CD33 IgG/kappa antibody HuM195 (Co et al., J. Immunol. 148:1149-1154, 1992; U.S. Pat. No. 5,693,761) and (ii) a single-chain Fv (scFv) form of HuTEC1 is attached at the penultimate glycine residue of CH3.

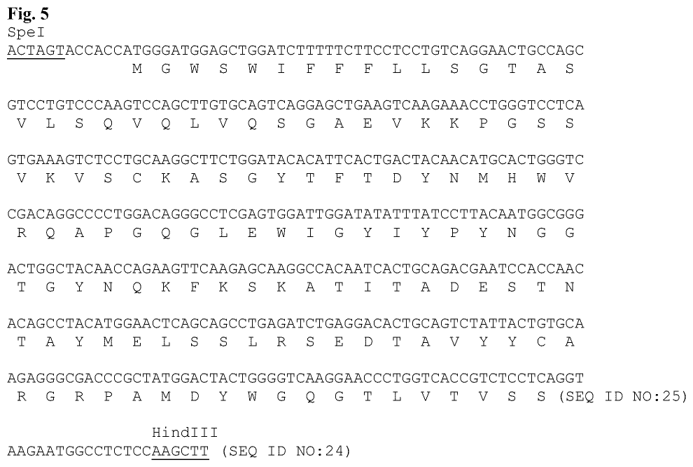

The HuM195 VH gene (SEQ ID NO:24) was synthesized as an exon including a splice donor signal, an SpeI site at the 5′ end, and a HindIII site at the 3′ end (FIG. 5). Amino acid sequence of HuM195 VH encoded in pHuM195.AA is MGWSWIFFFLLSGTASVLSQVQLVQSGAEVKKPGSSVKVSCKASGYTFTDYNMHWVR QAPGQGLEWIGYIYPYNGGTGYNQKFKSKATITADESTNTAYMELSSLRSEDTAVYYCA RGRPAMDYWGQGTLVTVSS (SEQ ID NO:25). Mature HuM195 VH starts at a glutamate residue at position 20 of SEQ ID NO:25.

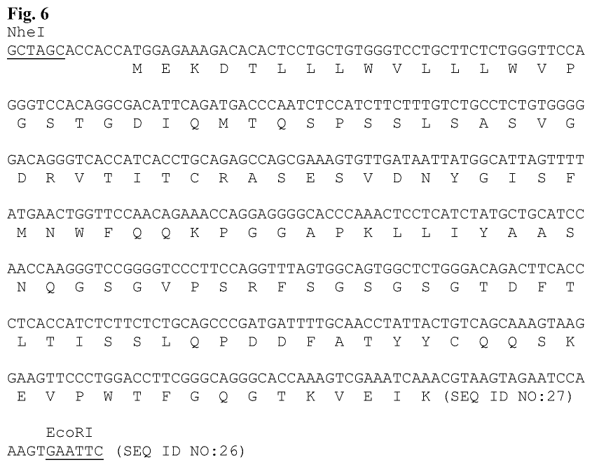

The HuM195 VL gene (SEQ ID NO:26) was synthesized as an exon including a splice donor signal, a NheI site at the 5′ end, and an EcoRI site at the 3′ end (FIG. 6). Amino acid sequence of HuM195 VL encoded in pHuM195.AA is MEKDTLLLWVLLLWVPGSTGDIQMTQSPSSLSASVGDRVTITCRASESVDNYGISFMNW FQQKPGGAPKLLIYAASNQGSGVPSRFSGSGSGTDFTLTISSLQPDDFATYYCQQSKEVP WTFGQGTKVEIK (SEQ ID NO:27). Mature HuM195 VL starts at an aspartate residue at position 21 of SEQ ID NO:27.

HuTEC1 VH and VL were converted to a scFv form in the order of N′-VL-flexible linker-VH-C′. In addition, two amino acid substitutions, one from alanine to cysteine at position 44 in HuTEC1 VH (FIG. 1A) and another from glycine to cysteine at position 100 in HuTEC1 VL (FIG. 1B), were introduced in such generated scFv. The amino acid sequence of the resultant HuTEC1 scFv termed HuTEC1.scFv.ds is

(SEQ ID NO: 28)
DIVMTQSPDSLAVSLGERATINCRASESVEYYGTSLMQWYQQKPGQPPKLLIYAASNVE
SGVPDRFSGSGSGTDFTLTISSLQAEDVAVYYCQQSRKVPWTFGCGTKVEIKGGGGSGG
GGSGGGGSQVTLRESGPALVKPTQTLTLTCTFSGFSLTGYGIYWVRQPPGKCLEWLGMI
WGSGSTDYNSALRSRLTISKDTSKNQVVLTMTNMDPVDTATYYCARDRGYYGSSSLYY
YAMDSWGQGTTVTVSS.

HuTEC1.scFv.ds was fused to the penultimate glycine residue of the CH3 region (SEQ ID NO:20) with a flexible polypeptide linker between them (CH3-HuTEC1.scFv.ds). The amino acid sequence of CH3-HuTEC1.scFv.ds is

(SEQ ID NO: 29)
GQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGSGGGGSGGGGS
DIVMTQSPDSLAVSLGERATINCRASESVEYYGTSLMQWYQQKPGQPPKLLIYAASNVE
SGVPDRFSGSGSGTDFTLTISSLQAEDVAVYYCQQSRKVPWTFGCGTKVEIKGGGGSGG
GGSGGGGSQVTLRESGPALVKPTQTLTLTCTFSGFSLTGYGIYWVRQPPGKCLEWLGMI
WGSGSTDYNSALRSRLTISKDTSKNQVVLTMTNMDPVDTATYYCARDRGYYGSSSLYY
YAMDSWGQGTTVTVSS.

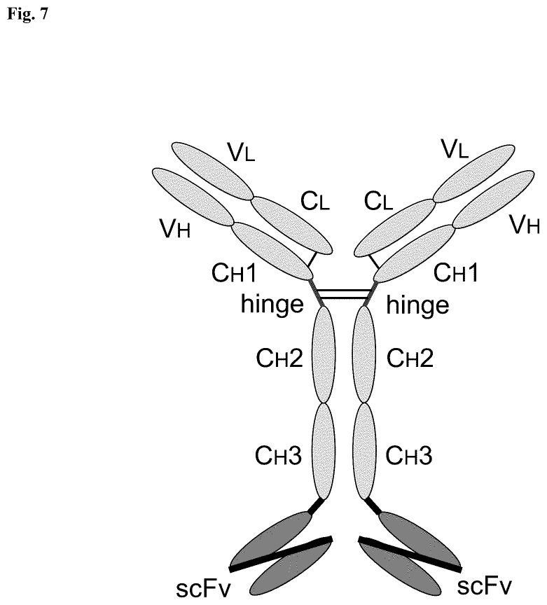

In the mammalian expression vector pBS959 (FIG. 4B), the coding region of CH3 (SEQ ID NO:20) in pHuTEC1.AA is replaced by that of CH3-HuTEC1.scFv.ds (SEQ ID NO:29). Bispecific anti-CD33/CD28 monoclonal antibody expressed from pBS959 was termed BS959. Schematic structure of BS959 is shown in FIG. 7.

Amino acid sequence of a mature heavy chain encoded in pBS959 is

(SEQ ID NO: 30)
QVQLVQSGAEVKKPGSSVKVSCKASGYTFTDYNMHWVRQAPGQGLEWIGYIYPYNGG
TGYNQKFKSKATITADESTNTAYMELSSLRSEDTAVYYCARGRPAMDYWGQGTLVTVS
SASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHTCPPCPAPEAAG
GPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSKLTVD
KSRWQQGNVFSCSVMHEALHNHYTQKSLSLSPGSGGGGSGGGGSDIVMTQSPDSLAVS
LGERATINCRASESVEYYGTSLMQWYQQKPGQPPKLLIYAASNVESGVPDRFSGSGSGT
DFTLTISSLQAEDVAVYYCQQSRKVPWTFGCGTKVEIKGGGGSGGGGSGGGGSQVTLR
ESGPALVKPTQTLTLTCTFSGFSLTGYGIYWVRQPPGKCLEWLGMIWGSGSTDYNSALR
SRLTISKDTSKNQVVLTMTNMDPVDTATYYCARDRGYYGSSSLYYYAMDSWGQGTTV
TVSS.

Amino acid sequence of a mature light chain encoded in pBS959 is

(SEQ ID NO: 31)
DIQMTQSPSSLSASVGDRVTITCRASESVDNYGISFMNWFQQKPGGAPKLLIYAASNQGS
GVPSRFSGSGSGTDFTLTISSLQPDDFATYYCQQSKEVPWTFGQGTKVEIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC.

### Example 4: Construction of a Bispecific Antibody That Binds to CD33 and CD3

SP34 is a mouse IgG3/lambda monoclonal agonist antibody that binds to human and cynomolgus CD3 epsilon protein. Cross-linking of CD3 epsilon proteins by SP34 on the surface of T cells induces activation of such T cells (Passano et al., EMBO J. 4:337-344, 1985; Yang et al., J. Immunol. 137:1097-1100, 1986; Salmerón et al., J. Immunol. 147:3047-3052, 1991; Perez-Aciego et al., J. Exp. Med. 174:319-326, 1991; Conrad et al., Cytom. A 71A: 925-933, 2007; U.S. Pat. Nos. 8,236,308 and 10,066,015). Amino acid sequence of the mature VH of SP34 is EVOLVESGGGLVQPKGSLKLSCAASGFTFNTYAMNWVRQAPGKGLEWVARIRSKYNN YATYYADSVKDRFTISRDDSQSILYLQMNNLKTEDTAMYYCVRHGNFGNSYVSWFAY WGQGTLVTVSS (SEQ ID NO:56). Amino acid sequence of the mature VL of SP34 is QAVVTQESALTTSPGETVTLTCRSSTGAVTTSNYANWVQEKPDHLFTGLIGGTNKRAPG VPARFSGSLIGDKAALTITGAQTEDEAIYFCALWYSNLWVFGGGTKLTVL (SEQ ID NO:57).

Humanized SP34 VH and VL amino acid sequences were designed following the general procedure of Tsurushita et al. (supra) as described in PCT/US2023/080133. The resulting humanized anti-CD3 IgG1 antibody termed HuSP34V maintained the affinity and specificity of the parental mouse SP34 antibody. Amino acid sequence of mature HuSP34V VH is EVOLVESGGGLVKPGGSLRLSCAASGFTFNTYAMNWVRQAPGKGLEWVARIRSKYNN YATYYADSVKDRFTISRDDSKNTLYLQMNSLKTEDTAVYYCVRHGNFGNSYVSWFAY WGQGTLVTVSS (SEQ ID NO:58). Amino acid sequence of mature HuSP34V VL is EIVLTQSPATLSLSPGERATLSCRSSTGAVTTSNYANWVQQKPGQAPRGLIGGTNKRAPG IPARFSGSLSGTDATLTISSLEPEDFAVYYCALWYSNLWVFGGGTKVEIK (SEQ ID NO:59).

HuSP34V.scFv.ds is a scFv form of HuSP34V that was constructed in the format of N′-VL-flexible linker-VH-C′ (PCT/US2023/080133). In HuSP34V.scFv.ds, each of two glycine residues at heavy chain position 44 and light chain position 100 was substituted by a cysteine residue. Amino acid sequence of HuSP34V.scFv.ds is

(SEQ ID NO: 32)
EIVLTQSPATLSLSPGERATLSCRSSTGAVTTSNYANWVQQKPGQAPRGLIGGTNKRAPG
IPARFSGSLSGTDATLTISSLEPEDFAVYYCALWYSNLWVFGCGTKVEIKGGGGSGGGGS
GGGGSEVQLVESGGGLVKPGGSLRLSCAASGFTFNTYAMNWVRQAPGKCLEWVARIR
SKYNNYATYYADSVKDRFTISRDDSKNTLYLQMNSLKTEDTAVYYCVRHGNFGNSYVS
WFAYWGQGTLVTVSS.

An expression vector for a bispecific antibody that binds to CD33 and CD3 has the replacement of the region encoding HuTEC1.scFv.ds (SEQ ID NO:28) with the region encoding HuSP34V.scFv.ds (SEQ ID NO:32) when compared to pBS959. The resultant expression vector named pJB564 has the same structure as pBS959 except for the scFv sequence attached to CH3. Schematic structure of pJB564 is shown in FIG. 4C. Bispecific anti-CD33/CD3 monoclonal antibody expressed from pJB564 was named JB564. Schematic structure of JB564 is shown in FIG. 7.

Amino acid sequence of a mature heavy chain encoded in pJB564 is

(SEQ ID NO: 33)
QVQLVQSGAEVKKPGSSVKVSCKASGYTFTDYNMHWVRQAPGQGLEWIGYIYPYNGG
TGYNQKFKSKATITADESTNTAYMELSSLRSEDTAVYYCARGRPAMDYWGQGTLVTVS
SASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHTCPPCPAPEAAG
GPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSKLTVD
KSRWQQGNVFSCSVMHEALHNHYTQKSLSLSPGSGGGGSGGGGSEIVLTQSPATLSLSP
GERATLSCRSSTGAVTTSNYANWVQQKPGQAPRGLIGGTNKRAPGIPARFSGSLSGTDA
TLTISSLEPEDFAVYYCALWYSNLWVFGCGTKVEIKGGGGSGGGGSGGGGSEVQLVES
GGGLVKPGGSLRLSCAASGFTFNTYAMNWVRQAPGKCLEWVARIRSKYNNYATYYAD
SVKDRFTISRDDSKNTLYLQMNSLKTEDTAVYYCVRHGNFGNSYVSWFAYWGQGTLV
TVSS.

Amino acid sequence of a mature light chain encoded in pJB564 is the same as the light chain sequence encoded in pBS959 (SEQ ID NO:31).

### Example 5: Construction of a Trispecific Antibody That Binds to CD33, CD28 and CD3

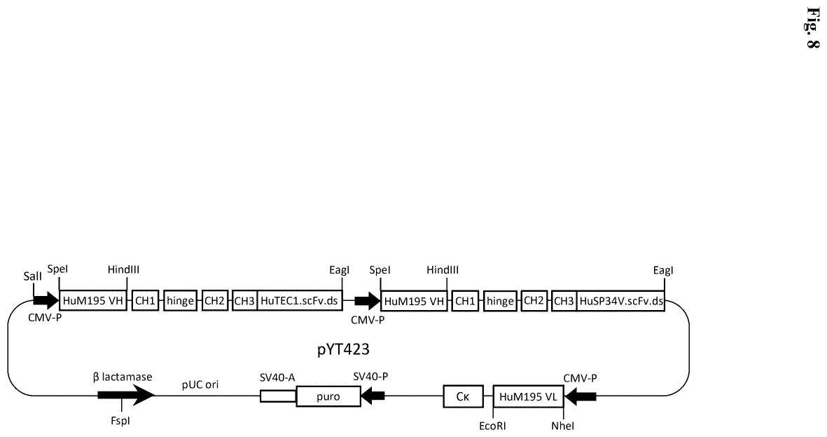

To construct an expression vector for production of a trispecific antibody that binds to CD33, CD28 and CD3, the heavy chain transcription unit encoded in pBS959, including the CMV promotor, HuM195VH, CH1, hinge, CH2 and CH3-HuTEC1.scFv.ds exons, and the polyadenylation site, was inserted upstream of the heavy chain transcription unit in pJB564. The resultant expression vector was named pYT423. Schematic structure of pYT423 is shown in FIG. 8. The vector pYT423 carries two heavy chain transcription units, one for a HuM195 heavy chain fused at the C-terminal to HuTEC1.scFv.ds (1^{st} heavy chain; SEQ ID NO:30) and another for a HuM195 heavy chain fused at the C-terminal to HuSP34V.scFv.ds (2nd heavy chain; SEQ ID NO:33) which is located downstream of the 1^{st} heavy chain transcription unit, and a light chain transcription unit that encodes a HuM 195 light chain (SEQ ID NO:31) which is located downstream of the 2^{nd} heavy chain transcription unit.

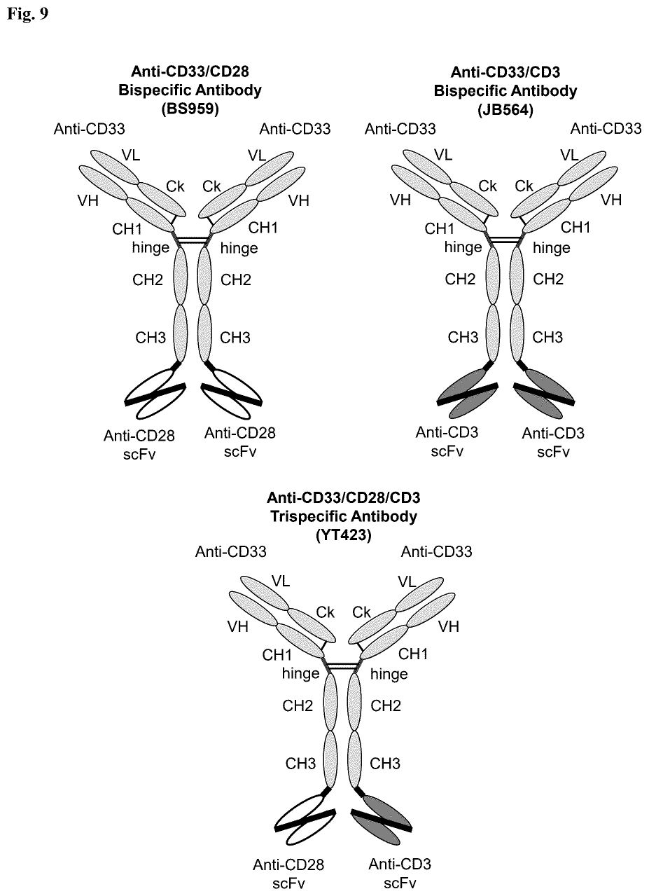

When pYT423 is introduced in mammalian cells, three types of antibodies are produced as shown in FIG. 9. The first type is BS959 that binds to CD33 and CD28, which comprises a homodimer of 1^{st} heavy chains. The second type is JB564 that binds to CD33 and CD3, which comprises a homodimer of 2^{nd} heavy chains. The third type, which was termed YT423, is composed of a 1^{st} heavy chain and a 2^{nd} heavy chain, which form a hetero dimer, and two HuM195 light chains. Schematic structure of YT423 is shown in FIG. 9. YT423 binds to CD33, CD28 and CD3.

### Example 6: Expression and Purification of JB564 and YT423

Generation of a CHO-K1 stable transfectant cell line producing JB564 (CHO-K1/pJB564), selection and expansion of CHO-K1/pJB564 cells producing a high level of JB564, and purification of JB564 from culture supernatants by protein A column chromatography were carried out as described above. SDS-PAGE analysis of purified JB564 under reducing conditions showed two predominant bands of the expected size (roughly 75 kDa heavy chain and 25 kDa light chain) of JB564. Analysis of purified JB564 with a Superose® 6 size exclusion column showed a single dominant peak of approximately 200 kDa.

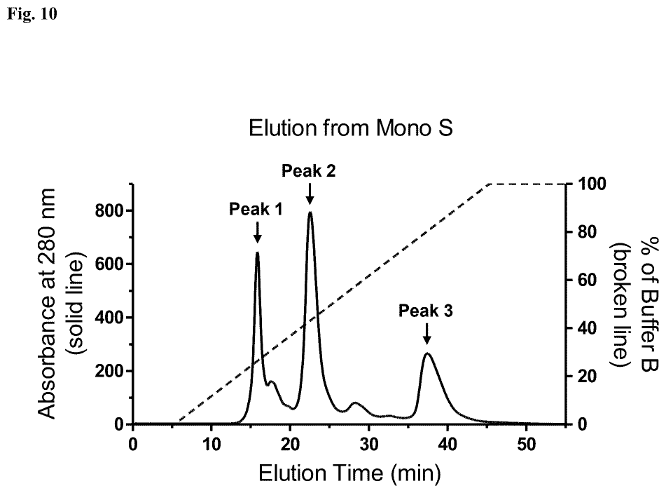

Stable transfection of CHO-K1 with pYT423, selection and expansion of CHO-K1 transfectants expressing a high level of IgG1/kappa antibodies (CHO-K1/pYT423), and purification of IgG1/kappa antibodies from culture supernatants of CHO-K1/pYT423 cells by protein A column chromatography were carried out as described above. For further purification of eluted antibodies, two types of buffers (Buffer A and Buffer B) were prepared. Buffer A contains 20 mM each of sodium citrate, sodium phosphate, Tris, and glycine with pH adjusted to 5.0. Buffer B contains 20 mM each of sodium citrate, sodium phosphate, Tris, and glycine with pH adjusted to 8.8. Eluted antibodies were dialyzed in Buffer A and loaded on a Mono S 5/50 GL cation exchange chromatography column (Cytiva). Antibodies bound to the Mono S column were eluted by linear gradient of pH from 5.0 (Buffer A) to 8.8 (Buffer B). The elution pattern is shown in FIG. 10. Three major peaks, which were named Peaks 1, 2, and 3 based on the order of elution as shown in FIG. 10, were collected separately.

SDS-PAGE analysis under reducing conditions showed that antibodies in each of Peaks 1, 2, and 3 comprise two predominant polypeptides, roughly 75 kid heavy chains and 25 kid light chains. Identity of the antibody in each of Peaks 1, 2, and 3 was analyzed by ELISA. An ELISA plate was coated with 1 μg/ml of Human CD3 epsilon & CD3 delta Heterodimer Protein, Fc Tag&Fc Tag (CD3-Fc; Acro Biosystems, Newark, DE), CD28-Fc (Sino Biological), or Recombinant Human Siglec 3-Fc Chimera (CD33-Fc; BioLegend) in PBS, blocked with ELISA Buffer, and used for incubation with antibodies in Peak 1, 2, or 3. Bound antibodies were detected with HRP-conjugated goat anti-human kappa chain antibody (Bethyl Laboratories) followed by color development with ABTS. Antibodies in Peak 1 showed binding to CD33-Fc and CD28-Fc, but not to CD3-Fc. Antibodies in Peak 2 showed binding to CD33-Fc, CD28-Fc and CD3-Fc. Antibodies in Peak 3 showed binding to CD33-Fc and CD3-Fc, but not to CD28-Fc.

SDS-PAGE analysis of antibodies in Peak 2 under reducing conditions showed only two predominant bands of the expected size (roughly 75 kDa heavy chains and 25 kDa light chains) of YT423. Analysis of antibodies in Peak 2 with a Superose® 6 size exclusion column showed a single dominant peak of approximately 200 kDa. These results indicated that the antibody in Peak 2 was a trispecific antibody YT423 binding to CD33, CD28 and CD3.

### Example 7: Biological Activities of JB564 and YT423

Activity of JB564 and YT423 to induce T cell-mediated cytotoxicity was analyzed with two human CD33-positive cell lines, acute promyelocytic leukemia HL-60 and acute monocytic leukemia THP-1. One hundred thousand of human peripheral blood mononuclear cells (PBMC) (effector cells), which had been incubated overnight in RPMI 1640 media containing 10% fetal bovine serum, 1 mM sodium pyruvate, 10 mM HEPES, 100 units/ml penicillin, and 100 μg/ml streptomycin (RPMI 1640 complete media) in a tissue culture plate to remove adherent cells, were mixed with ten thousand cells of HL-60 or THP-1 (target cells) labeled using the CellTrace™ Far Red Cell Proliferation Kit (Invitrogen, Waltham, MA) in 200 μl of RPMI 1640 complete media in wells of a 96-well plate. Test antibodies were added at 1 μg/ml. After 3 days of incubation at 37° C. in a 7.5% CO₂ incubator, cells were recovered from each well and treated with Helix NP Green (BioLegend, San Diego, CA) to stain dead cells. Fifty μl of Precision Count Beads (BioLegend) were then added to each cell sample to obtain absolute counts of cells at flow cytometry. Cells that are CellTrace™ Far Red-positive and Helix NP Green-negative were counted as living target cells (HL-60 or THP-1). Culture supernatants were saved for ELISA to measure the expression level of IFNγ, IL-2, and granzyme B.

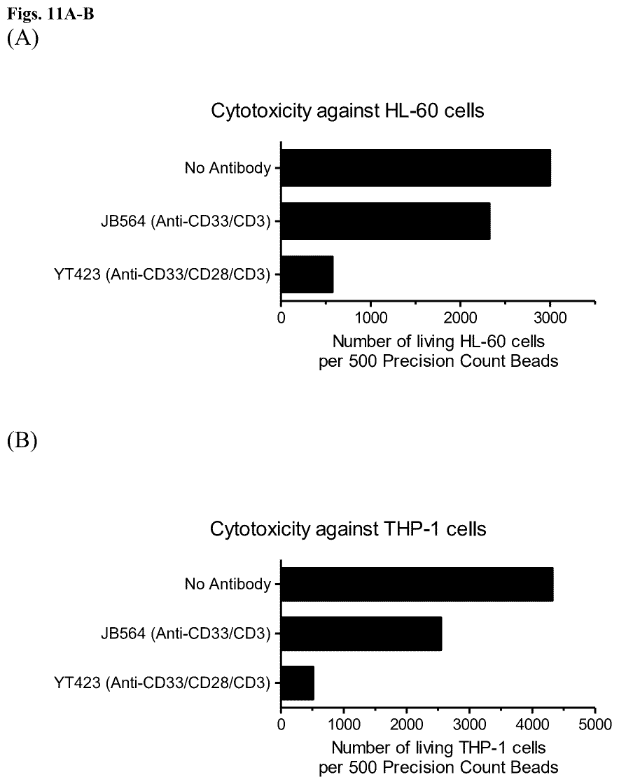

Number of living HL-60 or THP-1 cells was counted by flow cytometry. The data is shown in FIGS. 11A (for HL-60) and 11B (for THP-1). The numbers of living HL-60 cells after 3-day incubation per five hundred Precision Count Beads were (i) 2,999 without test antibodies, (ii) 2,323 with JB564 (anti-CD33/CD3 bispecific antibody), and (iii) 577 with YT423 (anti-CD33/CD28/CD3 trispecific antibody). The numbers of living THP-1 cells after 3-day incubation per five hundred Precision Count Beads were (iv) 4,323 without test antibodies, (v) 2,549 with JB564, and (vi) 514 with YT423. For both HL-60 and THP-1 cells, YT423 induced cell killing more efficiently than JB564 did.

Expression of IFNγ, IL-2, and granzyme B in culture supernatants was analyzed by ELISA using the ELISA MAX Standard Set Human IFN-γ kit (BioLegend), ELISA MAX™ Standard Set Human IL-2 kit (BioLegend), and ELISA Flex: Human Granzyme B (HRP) (Matich, Cincinnati, OH), respectively. The data is summarized in Table 4 shown below.

TABLE 4

| | Test | | | |
|---|---|---|---|---|
| Cells | Antibody | IFNγ | IL-2 | Granzyme B |
| HL-60 | None | Below | Below | Below |
| | | 156 pg/ml | 156 pg/ml | 625 pg/ml |
| | JB564 | Below | Below | Below |
| | | 156 pg/ml | 156 pg/ml | 625 pg/ml |
| | YT423 | 256 pg/ml | 662 pg/ml | 5,390 pg/ml |
| THP-1 | None | Below | Below | Below |
| | | 156 pg/ml | 156 pg/ml | 625 pg/ml |
| | JB564 | Below | 192 pg/ml | Below |
| | | 156 pg/ml | | 625 pg/ml |
| | YT423 | 515 pg/ml | 4,100 pg/ml | 9,890 pg/ml |

When no test antibody was added, expression of each of IFNγ, IL-2 and granzyme B was below the detection limit for both HL-60 and THP-1 cells. Trispecific anti-CD33/CD28/CD3 antibody YT423 induced expression of IFNγ, IL-2 and granzyme B much more efficiently with each of HL-60 and THP-1 than bispecific anti-CD33/CD3 antibody JB564 did.

### Example 8: Construction of an Anti-EGFR/CD3 Bispecific Antibody (JB559) and an Anti-EGFR/CD28/CD3 Trispecific Antibody (YT419)

An expression vector for a bispecific antibody that binds to epidermal growth factor receptor (EGFR) and CD3 was constructed in such a way that the VH and VL genes in pJB564 were substituted by those derived from a mouse anti-EGFR antibody 225 (225 VH and VL, respectively) (Masui et al. Cancer Res. 44:1002-1007, 1984; Gill et al. J. Biol. Chem. 259:7755-7760, 1984). The resultant vector was named pJB559. The vectors pJB564 and pJB559 share the same sequence except for the VH and VL regions. Bispecific anti-EGFR/CD3 IgG/kappa antibody termed JB559 is produced from pJB559 in mammalian cells. Schematic structure of JB559 is shown in FIG. 7.

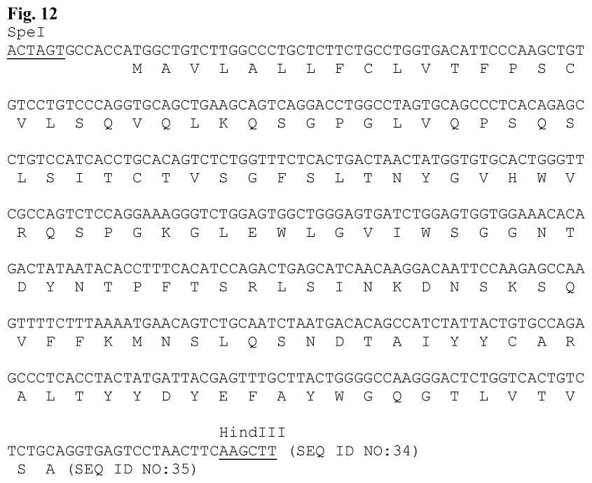

A gene encoding 225 VH (SEQ ID NO:34) was synthesized as an exon including a splice donor signal, a SpeI site at the 5′ end, and a HindIII site at the 3′ end (FIG. 12). Amino acid sequence of 225 VH encoded in pJB559 is

MAVLALLFCLVTFPSCVLSQVQLKQSGPGLVQPSQSLSITCTVSGFSLT
NYGVHWVRQSPGKGLEWLGVIWSGGNTDYNTPFTSRLSINKDNSKSQVF
FKMNSLQSNDTAIYYCARALTYYDYEFAYWGQGTLVTVSA (SEQ ID
NO: 35; https://go.drugbank.com/drugs/DB00002).

Mature 225 VH starts at a glutamate residue at position 20 of SEQ ID NO:35.

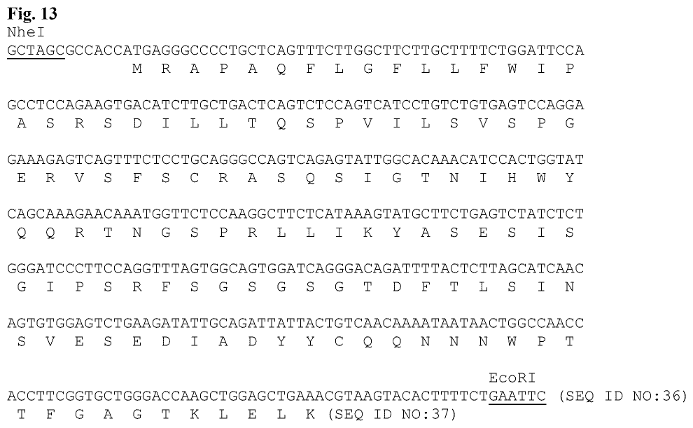

A gene encoding 225 VL (SEQ ID NO:36) was synthesized as an exon including a splice donor signal, a NheI site at the 5′ end, and an EcoRI site at the 3′ end (FIG. 13). Amino acid sequence of 225 VL encoded in pJB559 is MRAPAQFLGFLLFWIPASRSDILLTQSPVILSVSPGERVSFSCRASQSIGTNIHWYQQRTN GSPRLLIKYASESISGIPSRFSGSGSGTDFTLSINSVESEDIADYYCQQNNNWPTTFGAGTK LELK (SEQ ID NO:37; https://go.drugbank.com/drugs/DB00002). Mature 225 VL starts at an aspartate residue at position 21 of SEQ ID NO:37.

Amino acid sequence of a mature heavy chain encoded in pJB559 is

(SEQ ID NO: 38)
QVQLKQSGPGLVQPSQSLSITCTVSGFSLTNYGVHWVRQSPGKGLEWLGVIWSGGNTD
YNTPFTSRLSINKDNSKSQVFFKMNSLQSNDTAIYYCARALTYYDYEFAYWGQGTLVT
VSAASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPE
AAGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPGSGGGGSGGGGSEIVLTQSPAT
LSLSPGERATLSCRSSTGAVTTSNYANWVQQKPGQAPRGLIGGTNKRAPGIPARFSGSLS
GTDATLTISSLEPEDFAVYYCALWYSNLWVFGCGTKVEIKGGGGSGGGGSGGGGSEVQ
LVESGGGLVKPGGSLRLSCAASGFTFNTYAMNWVRQAPGKCLEWVARIRSKYNNYAT
YYADSVKDRFTISRDDSKNTLYLQMNSLKTEDTAVYYCVRHGNFGNSYVSWFAYWGQ
GTLVTVSS.

Amino acid sequence of a mature light chain encoded in pJB559 is

(SEQ ID NO: 39)
DILLTQSPVILSVSPGERVSFSCRASQSIGTNIHWYQQRTNGSPRLLIKYASESISGIPSRFS
GSGSGTDFTLSINSVESEDIADYYCQQNNNWPTTFGAGTKLELKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC.

A plasmid vector for expression of a trispecific antibody binding to EGFR, CD28 and CD3 has replacements of (i) each of the two HuM195 VH genes (SEQ ID NO:24) by the 225 VH gene (SEQ ID NO:34) and (ii) the HuM195 VL gene (SEQ ID NO:26) by the 225 VL gene (SEQ ID NO:36) in pYT423. The resultant vector was named pYT419. The structure of pYT419 is the same as that of pYT423 except for the VH and VL genes. Trispecific anti-EGFR/CD28/CD3 IgG/kappa antibody termed YT419 is produced from pYT419. Schematic structure of YT419 is shown in FIG. 7.

The expression vector pYT419 carries two heavy chain transcription units, one for a 225 heavy chain fused at the C-terminal to HuTEC1.scFv.ds (SEQ ID NO:40) and another for a 225 heavy chain fused at the C-terminal to HuSP34V.scFv.ds (SEQ ID NO:38), and one light chain transcription unit that encodes a 225 light chain (SEQ ID NO:39). Amino acid sequence of a mature 225 heavy chain fused to HuTEC1.scFv.ds encoded in pYT419 is

(SEQ ID NO: 40)
QVQLKQSGPGLVQPSQSLSITCTVSGFSLTNYGVHWVRQSPGKGLEWLGVIWSGGNTD
YNTPFTSRLSINKDNSKSQVFFKMNSLQSNDTAIYYCARALTYYDYEFAYWGQGTLVT
VSAASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPE
AAGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPGSGGGGSGGGGSDIVMTQSPDS
LAVSLGERATINCRASESVEYYGTSLMQWYQQKPGQPPKLLIYAASNVESGVPDRESGS
GSGTDFTLTISSLQAEDVAVYYCQQSRKVPWTFGCGTKVEIKGGGGSGGGGSGGGGSQ
VTLRESGPALVKPTQTLTLTCTFSGFSLTGYGIYWVRQPPGKCLEWLGMIWGSGSTDYN
SALRSRLTISKDTSKNQVVLTMTNMDPVDTATYYCARDRGYYGSSSLYYYAMDSWGQ
GTTVTVSS.

### Example 9: Characterization of JB559 and YT419

Generation of CHO-K1 stably transfected with each of pJB559 and pYT419 (CHO-K1/pJB559 and CHO-K1/pYT419, respectively) was carried out as described above. Purification of anti-EGFR/CD3 bispecific antibody JB559 from culture supernatants of CHO-K1/pJB559 cells were carried out as described above. Purification and fractionation of anti-EGFR/CD28/CD3 trispecific antibody YT419 was carried out using Mono S column chromatography after purification by Protein A column chromatography as described above. SDS-PAGE analysis of each of purified JB559 and YT419 under reducing conditions showed only two predominant bands of the expected size (roughly 75 kDa heavy chains and 25 kDa light chains). The analysis with a Superose® 6 size exclusion column showed a single dominant peak of approximately 200 kDa for each of JB559 and YT419.

The activity of JB559 and YT419 to induce T cell-mediated cytotoxicity was analyzed with EGFR-positive human colorectal adenocarcinoma HT-29 as target cells. HT-29 cells were first labeled using the CellTrace™ Far Red Cell Proliferation Kit, seeded in wells of a 96-well plate at ten thousand cells/well in RPMI 1640 complete media, and incubated overnight at 37° C. in a 7.5% CO2 incubator. Human PBMC, which had been incubated overnight in RPMI 1640 complete media in a tissue culture plate to remove adherent cells, were then added at one hundred thousand cells/well in a total volume of 200 μl RPMI 1640 complete media. Test antibodies were added at 1 μg/ml. After further incubation for 3 days, HT-29 cells and PBMC were recovered from each well and subjected to the analysis of viability. HT-29 cells attached to the well were recovered by treatment with PBS containing 0.05% trypsin and 0.5 mM EDTA. Culture supernatants were saved for the assay of cytokine release.

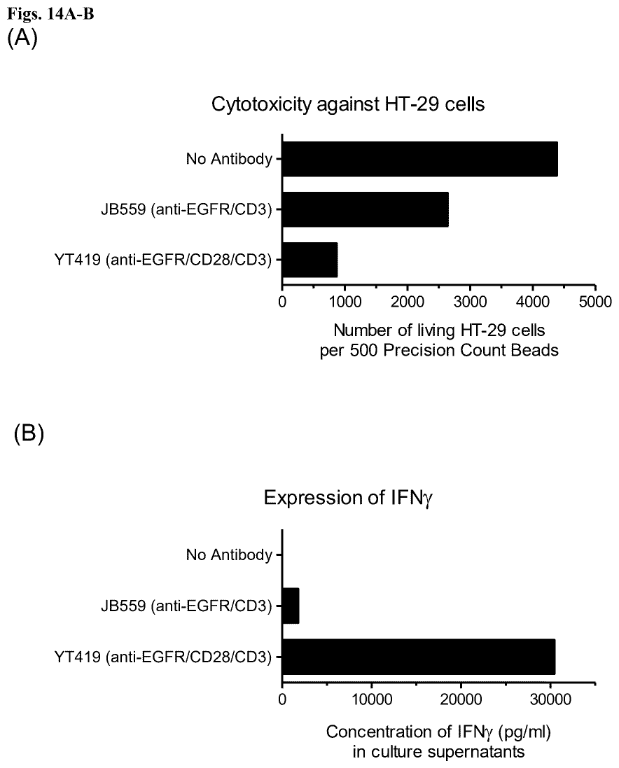

Cells recovered from each well were treated with Helix NP Green (BioLegend, San Diego, CA) to stain dead cells. Fifty μl of Precision Count Beads (BioLegend) were then added to each cell sample to obtain absolute counts of cells at flow cytometry. Cells that are CellTrace™ Far Red-positive and Helix NP Green-negative were counted as living HT-29 cells. The data is shown in FIG. 14A. The numbers of living HT-29 cells per five hundred Precision Count Beads were (i) 4,386 without test antibodies, (ii) 2,644 with JB559 (anti-EGFR/CD3 bispecific antibody), and (iii) 870 with YT419 (anti-EGFR/CD28/CD3 trispecific antibody). YT419 induced killing of HT-29 cells more efficiently than JB559 did.

The level of IFNγ in culture supernatants was measured as an indicator of T cell activation by ELSA using the ELISA MAX Standard Set Human IFN-γ kit (BioLegend). The result is shown in FIG. 14B. The IFNγ concentrations were (iv) below the detection limit of 156 pg/ml without test antibodies, (v) 1,810 pg/ml with JB559, and (vi) 30,430 pg/ml with YT419. Induction of IFNγ expression was much more efficient by YT419 than JB559.

Activation of T cells by JB559 and YT419 was analyzed in the absence of EGFR-expressing cells. One hundred thousand human PBMC were incubated in 200 μl RPMI 1640complete media in wells of a 96-well plate with no additional reagents, 1 μg/ml of JB559, 1 μg/ml of YT419, or one hundred thousand Human CD3/CD28 T Cell Activation Beads (Anti-CD3/CD28 Beads; BioLegend) as a positive control of activation of T cells. The levels of IFNγ and IL-2 in culture supernatants were measured by ELISA as described above. The IFNγ concentrations were (vii) below the detection limit of 156 pg/ml with no additional reagents, (viii) below 156 pg/ml with JB559, (ix) below 156 pg/ml with YT419, and (x) 2,533 pg/ml with Anti-CD3/CD28 Beads. The IL-2 concentrations were (xi) below the detection limit of 156 pg/ml with no additional reagents, (xii) below 156 pg/ml with JB559, (xiii) below 156 pg/ml with YT419, and (xiv) 11,835 pg/ml with Anti-CD3/CD28 Beads. Both JB559 and YT419 failed to induce IFNγ and IL-2 in PBMC without EGFR-expressing cells when compared to Anti-CD3/CD28 Beads.

### Example 10: Construction of Anti-CD20/CD3 Bispecific Antibody (JB554) and Anti-CD20/CD28/CD3 Trispecific Antibody (YT405)

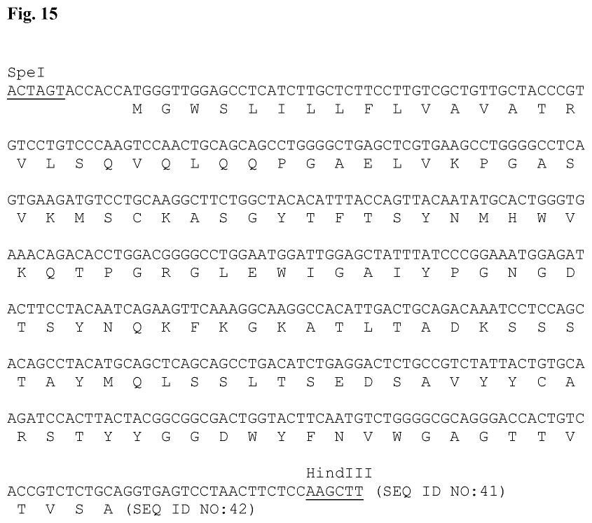

C2B8 is a chimeric anti-CD20 IgG/kappa antibody (Reff et al. Blood 83:435-445, 1994; Maloney et al. Blood 84:2457-2466, 1994). A gene encoding C2B8 VH was synthesized as an exon including a signal peptide, a splice donor signal, an SpeI site at the 5′ end, and a HindIII site at the 3′ end. The sequence of the C2B8 VH gene (SEQ ID NO:41) is shown in FIG. 15 along with the deduced amino acid sequence of C2B8 VH (SEQ ID NO:42). Mature C2B8 VH starts at a glutamate residue at position 20 of SEQ ID NO:42. Amino acid sequence of mature C2B8 VH encoded in pJB554 is

QVQLQQPGAELVKPGASVKMSCKASGYTFTSYNMHWVKQTPGRGLEWIG
AIYPGNGDTSYNQKFKGKATLTADKSSSTAYMQLSSLTSEDSAVYYCAR
STYYGGDWYFNVWGAGTTVTVSA (SEQ ID NO: 43;
https://go.drugbank.com/drugs/DB00073).

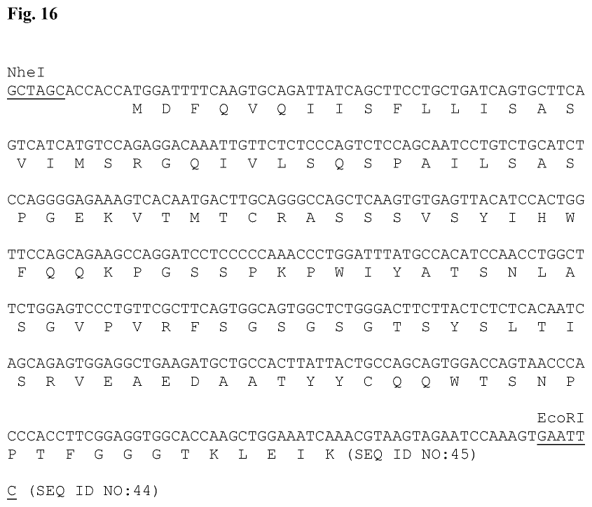

A gene encoding C2B8 VL was likewise synthesized as an exon including a signal peptide, a splice donor signal, an NheI site at the 5′ end, and an EcoRI site at the 3′ end. The sequence of the C2B8 VL gene (SEQ ID NO:44) is shown in FIG. 16 along with the deduced amino acid sequence of C2B8 VL (SEQ ID NO:45). Mature C2B8 VL starts at a glutamate residue at position 23 of SEQ ID NO:45. Amino acid sequence of mature C2B8 VL encoded in pJB554 and pYT405 is

QIVLSQSPAILSASPGEKVTMTCRASSSVSYIHWFQQKPGSSPKPWIYA
TSNLASGVPVRFSGSGSGTSYSLTISRVEAEDAATYYCQQWTSNPPTFG
GGTKLEIK (SEQ ID NO: 46; https://go.drugbank.com/
drugs/DB00073).

An expression vector for production of a bispecific anti-CD20/CD3 antibody was constructed in such a way that the HuM195 VH and VL genes in pJB564 were replaced by the C2B8 VH and VL genes, respectively. The resultant vector for expression of bispecific anti-CD20/CD3 antibody was named pJB554. The structure of pJB554 is the same as that of pJB564 (FIG. 4C) except that the HuM195 VH and VL genes are replaced by the C2B8 VH and VL genes, respectively, in pJB554. Bispecific anti-CD20/CD3 IgG/kappa antibody termed JB554 is produced from pJB554. Schematic structure of JB554 is shown in FIG. 7.

Amino acid sequence of a mature heavy chain encoded in pJB554 is

(SEQ ID NO: 47)
QVQLQQPGAELVKPGASVKMSCKASGYTFTSYNMHWVKQTPGRGLEWIGAIYPGNGD
TSYNQKFKGKATLTADKSSSTAYMQLSSLTSEDSAVYYCARSTYYGGDWYFNVWGAG
TTVTVSAASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDKTHTCPPCP
APEAAGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGSGGGGSGGGGSEIVLTQSP
ATLSLSPGERATLSCRSSTGAVTTSNYANWVQQKPGQAPRGLIGGTNKRAPGIPARFSGS
LSGTDATLTISSLEPEDFAVYYCALWYSNLWVFGCGTKVEIKGGGGSGGGGSGGGGSE
VQLVESGGGLVKPGGSLRLSCAASGFTFNTYAMNWVRQAPGKCLEWVARIRSKYNNY
ATYYADSVKDRFTISRDDSKNTLYLQMNSLKTEDTAVYYCVRHGNFGNSYVSWFAYW
GQGTLVTVSS.

Amino acid sequence of a mature light chain encoded in pJB554 is

(SEQ ID NO: 48)
QIVLSQSPAILSASPGEKVTMTCRASSSVSYIHWFQQKPGSSPKPWIYATSNLASGVPVRF
SGSGSGTSYSLTISRVEAEDAATYYCQQWTSNPPTFGGGTKLEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC

An expression vector for production of trispecific anti-CD20/CD28/CD3 antibody was constructed in such a way that (i) each of the two HuM195 VH genes was replaced by the C2B8 VH gene and (ii) the HuM195 VL gene was replaced by the C2B8 VL gene in pYT423. The resultant vector for expression of trispecific anti-CD20/CD28/CD3 antibody was named pYT405. The structure of pYT405 is the same as that of pYT423 (FIG. 8) except that the HuM195 VH and VL genes are replaced by the C2B8 VH and VL genes, respectively, in pYT405. Trispecific anti-CD20/CD28/CD3 IgG/kappa antibody termed YT405 is produced from pYT405. Schematic structure of YT405 is shown in FIG. 7.

The expression vector pYT405 carries two heavy chain transcription units, one for a C2B8 heavy chain fused at the C-terminal to HuTEC1.scFv.ds (SEQ ID NO:49) and another for a C2B8 heavy chain fused at the C-terminal to HuSP34V.scFv.ds (SEQ ID NO:47), and one light chain transcription unit that encodes a C2B8 light chain (SEQ ID NO:48). Amino acid sequence of a mature C2B8 heavy chain fused to HuTEC1.scFv.ds encoded in pYT405 is

(SEQ ID NO: 49)
QVQLQQPGAELVKPGASVKMSCKASGYTFTSYNMHWVKQTPGRGLEWIGAIYPGNGD
TSYNQKFKGKATLTADKSSSTAYMQLSSLTSEDSAVYYCARSTYYGGDWYFNVWGAG
TTVTVSAASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDKTHTCPPCP
APEAAGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGSGGGGSGGGGSDIVMTQSP
DSLAVSLGERATINCRASESVEYYGTSLMQWYQQKPGQPPKLLIYAASNVESGVPDRES
GSGSGTDFTLTISSLQAEDVAVYYCQQSRKVPWTFGCGTKVEIKGGGGSGGGGSGGGG
SQVTLRESGPALVKPTQTLTLTCTFSGFSLTGYGIYWVRQPPGKCLEWLGMIWGSGSTD
YNSALRSRLTISKDTSKNQVVLTMTNMDPVDTATYYCARDRGYYGSSSLYYYAMDSW
GQGTTVTVSS.

### Example 11: Characterization of JB554 and YT405

Generation of CHO-K1 stable transfectants with each of pJB554 and pYT405 (CHO-K1/pJB554 and CHO-K1/pYT405, respectively) was carried out as described above. Purification of anti-CD20/CD3 bispecific antibody JB554 from culture supernatants of CHO-K1/pJB554 cells by protein A chromatography was carried out as described above. Purification and fractionation of anti-CD20/CD28/CD3 trispecific antibody YT405 from culture supernatants of CHO-K1/pYT405 cells by protein A and Mono S chromatography was carried out as described above. SDS-PAGE analysis of each of purified JB554 and YT405 under reducing conditions showed only two predominant bands of the expected size (roughly 75 kDa heavy chains and 25 kDa light chains). The analysis with a Superose® 6 size exclusion column showed a single dominant peak of approximately 200 kDa with each of JB554 and YT405.

Activation of T cells by JB554 and YT405 was analyzed with human CD20-positive Burkitt's lymphoma cell line Ramos. Human T cells were isolated from PBMC using MojoSort™ Human CD3 T Cell Isolation Kit (BioLegend). Ramos cells were labeled using the CellTrace™ Far Red Cell Proliferation Kit. Fifty thousand human T cells were mixed with ten thousand labeled Ramos cells in 200 μl of RPMI 1640 complete media in wells of a 96-well plate. Test antibodies were added at 1 μg/ml. Cells were incubated for 3 days at 37° C. in a 7.5% CO₂ incubator.

Human T cells and Ramos cells were recovered as a mixture from each well and treated with Helix NP Green (BioLegend, San Diego, CA) to stain dead cells. Fifty μl of Precision Count Beads (BioLegend) were added to each cell sample to obtain absolute counts of cells at flow cytometry. Cells that are negative for fluorescence of both CellTrace™ Far Red and Helix NP Green were counted as living T cells. The numbers of living T cells per five hundred Precision Count Beads were (i) 3,796 without test antibodies, (ii) 4,729 with JB554, and (iii) 9,947 with YT405. Expression of IFNγ in culture supernatants of each well was analyzed by ELISA as described above. The levels of IFNγ were (iv) below the detection limit of 780 pg/ml without test antibodies, (v) below 780 pg/ml with JB554, and (vi) 3,650 pg/ml with YT405. These results indicated that trispecific anti-CD20/CD28/CD3 antibody YT405 activated T cells in the presence of CD20-positive Ramos cells more potently than bispecific anti-CD20/CD3 antibody JB554 did.

Activation of T cells by each of JB554 and YT405 was analyzed in the absence of CD20-expressing cells. Fifty thousand T cells isolated from human PBMC as described above were incubated with (or without) 1 μg/ml of a test sample (JB554 or YT405) in 200 μl of RPMI 1640 complete media in wells of a 96-well plate. As a positive control of activation of T cells, fifty thousand Human CD3/CD28 T Cell Activation Beads (Anti-CD3/CD28 Beads; BioLegend) were added instead of a test sample. Cells were incubated for 3 days at 37° C. in a 7.5% CO₂ incubator. The levels of IFNγ and IL-2 in culture supernatants were measured by ELISA as described above. The result is summarized in Table 5 shown below.

TABLE 5

| Test sample | IFNγ | IL-2 |
|---|---|---|
| None | 181 pg/ml | Below 156 pg/ml |
| JB554 | Below 156 pg/ml | Below 156 pg/ml |
| YT405 | Below 156 pg/ml | Below 156 pg/ml |
| Anti-CD3/CD28 Beads | 4,573 pg/ml | 2,006 pg/ml |

The expression level of each of IFNγ and IL-2 was below the detection limit of 156 pg/ml with each of JB554 and YT405. When compared to the effects of Anti-CD3/CD28 Beads on expression of IFNγ and IL-2 in culture supernatants (4,573 pg/ml and 2,006 pg/ml, respectively) and to the background level of IFNγ and IL-2 expression without any test sample (181 pg/ml and below the detection limit of 156 pg/ml, respectively), both JB554 and YT405 showed no sign of T cell activation in the expression levels of IFNγ and IL-2.

### Example 12: Construction, Expression, and Characterization of Anti-EGFR/CD28/CD3 Trispecific Antibody With the Knobs-Into-Holes Mutations (YT437)

The vector pYT419 for expression of anti-EGFR/CD28/CD3 trispecific antibody binding to EGFR, CD28 and CD3 (YT419) was modified in the CH3 regions to introduce the knobs-into-holes mutations (Ridgway et al., Protein Eng 9:617-21, 1996; Atwell et al., J Mol Biol 270:26-35, 1997; and U.S. Pat. No. 7,695,936) to facilitate the formation of heterodimers of anti-EGFR/CD28 and anti-EGFR/CD3 heavy chains. The mature 225 heavy chain fused to HuTEC1.scFv.ds (SEQ ID NO:40), which is encoded in pYT419, was modified by replacing the CH3 region (SEQ ID NO:20) with a variant CH3 region carrying amino acid substitutions from threonine to serine at position 366 (T366S), leucine to alanine at position 368 (L368A), and tyrosine to valine at position 407 (Y407V) (hole mutation) (SEQ ID NO:80). The mature 225 heavy chain fused to HuSP34V.scFv.ds (SEQ ID NO:38), which is encoded in pYT419 and also in pJB559, was modified by replacing the CH3 region (SEQ ID NO:20) with another variant CH3 region carrying an amino acid substitution from threonine to tryptophan at position 366 (T366W) (knob mutation) (SEQ ID NO:81). The resulting pYT419-derived vector, which was named pYT437, expresses two kinds of mature heavy chains (SEQ ID NOS: 80 and 81) and a single kind of mature light chain (SEQ ID NO:39). Trispecific anti-EGFR/CD28/CD3 antibody with the knobs-into-holes mutations termed YT437 is expressed from pYT437 in a cell.

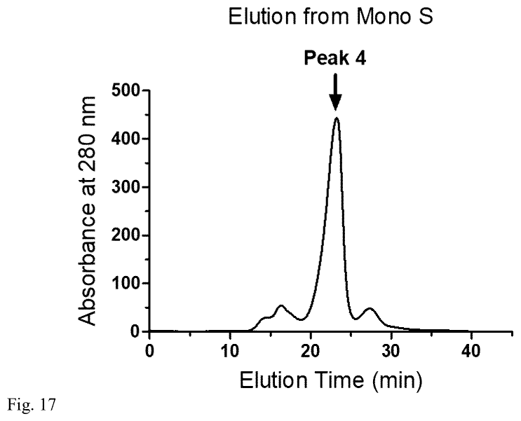

Generation of CHO-K1 cells stably transfected with pYT437 (CHO-K1/pYT437) was carried out as described above. Purification and fractionation of anti-EGFR/CD28/CD3 trispecific antibody YT437 from culture supernatants of CHO-K1/pYT437 cells was carried out using Protein A column chromatography followed by Mono S column chromatography as described above. The elution pattern of Mono S column chromatography is shown in FIG. 17. The single dominant peak (Peak 4) in FIG. 17 was collected and dialyzed against PBS. SDS-PAGE analysis of dialyzed antibodies in Peak 4 under reducing conditions showed only two predominant bands of the expected size for YT437 (roughly 75 kDa heavy chains and 25 kDa light chains). Analysis of antibodies in Peak 4 with a Superose® 6 size exclusion column showed a single dominant peak of approximately 200 kDa, which is the expected size of YT437. Antibodies in Peak 4 bound to EGFR, CD28 and CD3 in ELISA. Based on these analyses, the antibody present in Peak 4 was determined to be anti-EGFR/CD28/CD3 trispecific antibody YT437.

The activity of YT437 to induce T cell-mediated cytotoxicity was analyzed with EGFR-positive HT-29 cells as described above. HT-29 cells labeled with CellTrace™ Far Red were incubated with human PBMC at the E:T ratio of 10:1 with or without 1 μg/ml of YT437 in RPMI 1640 complete media for 3 days at 37° C. in a 7.5% CO₂ incubator. HT-29 cells and PBMC were recovered from each well and subjected to the analysis of viability as described above. Culture supernatants were saved for the assay of cytokine release.

When compared to the number of living HT-29 cells incubated with PBMC for 3 days without YT437 (100% viability control), the viability of HT-29 cells incubated with PBMC and YT437 was 6.1%. The concentrations of granzyme B and IFNγ in culture supernatants were measured by ELISA as described above. The expression level of granzyme B was less than the detection limit of 0.39 ng/ml in the absence of YT437, while it increased to 102 ng/ml in the presence of YT437. The expression level of IFNγ was less than the detection limit of 0.08 ng/ml without YT437 and 11.6 ng/ml with YT437. These results indicate that YT437 can induce potent cytotoxic activity of T cells against EGFR-expressing HT-29 cells.

SEQUENCE LISTING

SEQ ID NO: 1
Amino acid sequence of the extracellular region of human, cynomolgus, and rhesus
CD28
NKILVKQSPMLVAYDNAVNLSCKYSYNLFSREFRASLHKGLDSAVEVCVVY
GNYSQQLQVYSKTGFNCDGKLGNESVTFYLQNLYVNQTDIYFCKIEVMYPPPYLDNEK
SNGTIIHVKGKHLCPSPLFPGPSKP
SEQ ID NO: 2
Amino acid sequence of TEC1 VH
MAVLALLFCLVTFPSCILSQVQLKESGPGLVAPSQSLSITCTVSGFSLTGYGIY
WVRQPPGKGLEWLGMIWGDGSTDYNSALRSRLSINKDNSKSQVFLKMNSLQTDDTAR
YYCARDRGYYGSSSLYYYAMDSWGQGTSVTVSS
SEQ ID NO: 3
Amino acid sequence of CDRH1 of TEC1, HuTEC1 and HuTEC1.scFv.ds VH
GYGIY
SEQ ID NO: 4
Amino acid sequence of CDRH2 of TEC1 VH
MIWGDGSTDYNSALRS
SEQ ID NO: 5
Amino acid sequence of CDRH3 of TEC1, HuTEC1 and HuTEC1.scFv.ds VH
DRGYYGSSSLYYYAMDS
SEQ ID NO: 6
Amino acid sequence of TEC1 VL
MESDTLLLWVLLLWVPGSTGDIVLTQSPASLAVSLGQRATISCRASESVEYYG
TSLMQWYQQKPGQPPKLLIYAASNVESGVPARFSGSGSGTDFSLNIHPVEEDDIAMYFC
QQSRKVPWTFGGGTKLEIK
SEQ ID NO: 7
Amino acid sequence of CDRLI of TEC1, HuTEC1 and HuTEC1.scFv.ds VL
RASESVEYYGTSLMQ
SEQ ID NO: 8
Amino acid sequence of CDRL2 of TEC1 HuTEC1 and HuTEC1.scFv.ds VL
AASNVES
SEQ ID NO: 9
Amino acid sequence of CDRL3 of TEC1 HuTEC1 and HuTEC1.scFv.ds VL
QQSRKVPWT
SEQ ID NO: 10
Amino acid sequence of HuTEC1 VH
MAVLALLFCLVTFPSCILSQVTLRESGPALVKPTQTLTLTCTFSGFSLTGYGIY
WVRQPPGKALEWLGMIWGSGSTDYNSALRSRLTISKDTSKNQVVLTMTNMDPVDTAT
YYCARDRGYYGSSSLYYYAMDSWGQGTTVTVSS
SEQ ID NO: 11
Amino acid sequence of mature HuTEC1 VH
QVTLRESGPALVKPTQTLTLTCTFSGFSLTGYGIYWVRQPPGKALEWLGMIW
GSGSTDYNSALRSRLTISKDTSKNQVVLTMTNMDPVDTATYYCARDRGYYGSSSLYYY
AMDSWGQGTTVTVSS
SEQ ID NO: 12
Amino acid sequence of CDRH2 of HuTEC1 and HuTEC1.scFv.ds VH
MIWGSGSTDYNSALRS
SEQ ID NO: 13
Amino acid sequence of HuTEC1 VL
MESDTLLLWVLLLWVPGSTGDIVMTQSPDSLAVSLGERATINCRASESVEYY
GTSLMQWYQQKPGQPPKLLIYAASNVESGVPDRFSGSGSGTDFTLTISSLQAEDVAVYY
CQQSRKVPWTFGGGTKVEIK
SEQ ID NO: 14
Amino acid sequence of mature HuTEC1 VL
DIVMTQSPDSLAVSLGERATINCRASESVEYYGTSLMQWYQQKPGQPPKLLI
YAASNVESGVPDRFSGSGSGTDFTLTISSLQAEDVAVYYCQQSRKVPWTFGGGTKVEIK
SEQ ID NO: 15
DNA sequence of the exon encoding HuTEC1 VH
ACTAGTACCACCATGGCTGTCCTGGCACTTCTCTTCTGCCTGGTCACATTC
CCAAGCTGTATCCTTTCCCAAGTCACTCTGAGGGAGTCAGGACCTGCCCTTGTGAAG
CCCACACAGACCCTGACCCTCACATGCACCTTCTCAGGGTTCTCACTGACCGGCTAT
GGAATCTACTGGGTTCGCCAGCCTCCAGGAAAGGCTCTGGAGTGGCTGGGAATGAT
CTGGGGAAGTGGAAGCACAGACTATAATTCAGCTCTCAGATCCAGACTGACCATCA
GCAAGGACACCTCCAAGAACCAAGTTGTCCTGACAATGACCAATATGGACCCTGTT
GACACAGCCACCTACTACTGTGCCAGAGATCGGGGATACTACGGAAGTAGCTCCCT
GTATTACTATGCTATGGACTCCTGGGGACAAGGAACCACAGTCACCGTCTCCTCAGG
TAAGTATGGCCTCTCAAGCTT
SEQ ID NO: 16
DNA sequence of the exon encoding HuTEC1 VL
GCTAGCACCACCATGGAGTCAGACACACTCCTGCTTTGGGTTCTGCTGCTC
TGGGTTCCAGGCTCCACTGGAGACATTGTGATGACCCAATCTCCAGATTCTTTGGCT
GTGTCTCTTGGCGAAAGAGCCACCATCAACTGCAGAGCCAGTGAAAGTGTTGAATA
CTATGGCACAAGTCTGATGCAGTGGTACCAACAGAAACCAGGACAGCCACCCAAAC
TCCTCATCTATGCTGCATCCAATGTCGAATCTGGGGTCCCTGACAGATTTAGTGGCA
GTGGCTCTGGGACAGACTTCACCCTCACCATCAGTTCTCTGCAGGCCGAGGATGTTG
CAGTGTATTACTGTCAGCAAAGTAGGAAGGTTCCATGGACCTTCGGAGGAGGCACC
AAAGTTGAAATCAAACGTAAGTAGAATCCAAAGAATTC
SEQ ID NO: 17
Amino acid sequence of the CH1 region of human gamma-1 heavy chain encoded in
pHuTEC1.AA
ASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKV
SEQ ID NO: 18
Amino acid sequence of the hinge region of human gamma-1 heavy chain encoded in
pHuTEC1.AA
EPKSCDKTHTCPPCP
SEQ ID NO: 19
Amino acid sequence of the CH2 region of human gamma-1 heavy chain encoded in
pHuTEC1.AA
APEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAK
SEQ ID NO: 20
Amino acid sequence of the CH3 region of human gamma-1 heavy chain encoded in
pHuTEC1.AA
GQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGK
SEQ ID NO: 21
Amino acid sequences of the human kappa constant region (Cκ) encoded in
pHuTEC1.AA
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 22
Amino acid sequence of a mature heavy chain encoded in pHuTEC1.AA
QVTLRESGPALVKPTQTLTLTCTFSGFSLTGYGIYWVRQPPGKALEWLGMIWGSGSTDY
NSALRSRLTISKDTSKNQVVLTMTNMDPVDTATYYCARDRGYYGSSSLYYYAMDSWG
QGTTVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHTCP
PCPAPEAAGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 23
Amino acid sequence of a mature light chain encoded in pHuTEC1.AA
DIVMTQSPDSLAVSLGERATINCRASESVEYYGTSLMQWYQQKPGQPPKLLI
YAASNVESGVPDRESGSGSGTDFTLTISSLQAEDVAVYYCQQSRKVPWTFGGGTKVEIK
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 24
DNA sequence of the HuM195 VH gene
ACTAGTACCACCATGGGATGGAGCTGGATCTTTTTCTTCCTCCTGTCAGGA
ACTGCCAGCGTCCTGTCCCAAGTCCAGCTTGTGCAGTCAGGAGCTGAAGTCAAGAA
ACCTGGGTCCTCAGTGAAAGTCTCCTGCAAGGCTTCTGGATACACATTCACTGACTA
CAACATGCACTGGGTCCGACAGGCCCCTGGACAGGGCCTCGAGTGGATTGGATATA
TTTATCCTTACAATGGCGGGACTGGCTACAACCAGAAGTTCAAGAGCAAGGCCACA
ATCACTGCAGACGAATCCACCAACACAGCCTACATGGAACTCAGCAGCCTGAGATC
TGAGGACACTGCAGTCTATTACTGTGCAAGAGGGCGACCCGCTATGGACTACTGGG
GTCAAGGAACCCTGGTCACCGTCTCCTCAGGTAAGAATGGCCTCTCCAAGCTT
SEQ ID NO: 25
Amino acid sequence of HuM195 VH
MGWSWIFFFLLSGTASVLSQVQLVQSGAEVKKPGSSVKVSCKASGYTFTDY
NMHWVRQAPGQGLEWIGYIYPYNGGTGYNQKFKSKATITADESTNTAYMELSSLRSED
TAVYYCARGRPAMDYWGQGTLVTVSS
SEQ ID NO: 26
DNA sequence of the HuM195 VL gene
GCTAGCACCACCATGGAGAAAGACACACTCCTGCTGTGGGTCCTGCTTCT
CTGGGTTCCAGGGTCCACAGGCGACATTCAGATGACCCAATCTCCATCTTCTTTGTCT
GCCTCTGTGGGGGACAGGGTCACCATCACCTGCAGAGCCAGCGAAAGTGTTGATAA
TTATGGCATTAGTTTTATGAACTGGTTCCAACAGAAACCAGGAGGGGCACCCAAACT
CCTCATCTATGCTGCATCCAACCAAGGGTCCGGGGTCCCTTCCAGGTTTAGTGGCAG
TGGCTCTGGGACAGACTTCACCCTCACCATCTCTTCTCTGCAGCCCGATGATTTTGCA
ACCTATTACTGTCAGCAAAGTAAGGAAGTTCCCTGGACCTTCGGGCAGGGCACCAA
AGTCGAAATCAAACGTAAGTAGAATCCAAAGTGAATTC
SEQ ID NO: 27
Amino acid sequence of HuM195 VL
MEKDTLLLWVLLLWVPGSTGDIQMTQSPSSLSASVGDRVTITCRASESVDNY
GISFMNWFQQKPGGAPKLLIYAASNQGSGVPSRFSGSGSGTDFTLTISSLQPDDFATYYC
QQSKEVPWTFGQGTKVEIK
SEQ ID NO: 28
Amino acid sequence of HuTEC1.scFv.ds
DIVMTQSPDSLAVSLGERATINCRASESVEYYGTSLMQWYQQKPGQPPKLLI
YAASNVESGVPDRESGSGSGTDFTLTISSLQAEDVAVYYCQQSRKVPWTFGCGTKVEIK
GGGGSGGGGSGGGGSQVTLRESGPALVKPTQTLTLTCTFSGFSLTGYGIYWVRQPPGKC
LEWLGMIWGSGSTDYNSALRSRLTISKDTSKNQVVLTMTNMDPVDTATYYCARDRGY
YGSSSLYYYAMDSWGQGTTVTVSS
SEQ ID NO: 29
Amino acid sequence of CH3-HuTEC1.scFv.ds
GQPREPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSLSLSPGSGGG
GSGGGGSDIVMTQSPDSLAVSLGERATINCRASESVEYYGTSLMQWYQQKPGQPPKLLI
YAASNVESGVPDRESGSGSGTDFTLTISSLQAEDVAVYYCQQSRKVPWTFGCGTKVEIK
GGGGSGGGGSGGGGSQVTLRESGPALVKPTQTLTLTCTFSGFSLTGYGIYWVRQPPGKC
LEWLGMIWGSGSTDYNSALRSRLTISKDTSKNQVVLTMTNMDPVDTATYYCARDRGY
YGSSSLYYYAMDSWGQGTTVTVSS
SEQ ID NO: 30
Amino acid sequence of a mature heavy chain encoded in pBS959
QVQLVQSGAEVKKPGSSVKVSCKASGYTFTDYNMHWVRQAPGQGLEWIGY
IYPYNGGTGYNQKFKSKATITADESTNTAYMELSSLRSEDTA VYYCARGRPAMDYWGQ
GTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSCDKTHTCPPC
PAPEAAGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGSGGGGSGGGGSDIVMTQS
PDSLAVSLGERATINCRASESVEYYGTSLMQWYQQKPGQPPKLLIYAASNVESGVPDRF
SGSGSGTDFTLTISSLQAEDVAVYYCQQSRKVPWTFGCGTKVEIKGGGGSGGGGSGGG
GSQVTLRESGPALVKPTQTLTLTCTFSGFSLTGYGIYWVRQPPGKCLEWLGMIWGSGST
DYNSALRSRLTISKDTSKNQVVLTMTNMDPVDTATYYCARDRGYYGSSSLYYYAMDS
WGQGTTVTVSS
SEQ ID NO: 31
Amino acid sequence of a mature light chain encoded in pBS959 and pJB564
DIQMTQSPSSLSASVGDRVTITCRASESVDNYGISFMNWFQQKPGGAPKLLIY
AASNQGSGVPSRFSGSGSGTDFTLTISSLQPDDFATYYCQQSKEVPWTFGQGTKVEIKRT
VAAPSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
SEQ ID NO: 32
Amino acid sequence of HuSP34V.scFv.ds
EIVLTQSPATLSLSPGERATLSCRSSTGAVTTSNYANWVQQKPGQAPRGLIGG
TNKRAPGIPARFSGSLSGTDATLTISSLEPEDFAVYYCALWYSNLWVFGCGTKVEIKGGG
GSGGGGSGGGGSEVQLVESGGGLVKPGGSLRLSCAASGFTFNTYAMNWVRQAPGKCL
EWVARIRSKYNNYATYYADSVKDRFTISRDDSKNTLYLQMNSLKTEDTAVYYCVRHG
NFGNSYVSWFAYWGQGTLVTVSS
SEQ ID NO: 33
Amino acid sequence of a mature heavy chain encoded in pJB564
QVQLVQSGAEVKKPGSSVKVSCKASGYTFTDYNMHWVRQAPGQGLEWIGY
IYPYNGGTGYNQKFKSKATITADESTNTAYMELSSLRSEDTAVYYCARGRPAMDYWGQ
GTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSCDKTHTCPPC
PAPEAAGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGSGGGGSGGGGSEIVLTQS
PATLSLSPGERATLSCRSSTGAVTTSNYANWVQQKPGQAPRGLIGGTNKRAPGIPARFSG
SLSGTDATLTISSLEPEDFAVYYCALWYSNLWVFGCGTKVEIKGGGGSGGGGSGGGGSE
VQLVESGGGLVKPGGSLRLSCAASGFTFNTYAMNWVRQAPGKCLEWVARIRSKYNNY
ATYYADSVKDRFTISRDDSKNTLYLQMNSLKTEDTAVYYCVRHGNFGNSYVSWFAYW
GQGTLVTVSS
SEQ ID NO: 34
DNA sequence of the 225 VH gene
ACTAGTGCCACCATGGCTGTCTTGGCCCTGCTCTTCTGCCTGGTGACATTC
CCAAGCTGTGTCCTGTCCCAGGTGCAGCTGAAGCAGTCAGGACCTGGCCTAGTGCA
GCCCTCACAGAGCCTGTCCATCACCTGCACAGTCTCTGGTTTCTCACTGACTAACTAT
GGTGTGCACTGGGTTCGCCAGTCTCCAGGAAAGGGTCTGGAGTGGCTGGGAGTGAT
CTGGAGTGGTGGAAACACAGACTATAATACACCTTTCACATCCAGACTGAGCATCA
ACAAGGACAATTCCAAGAGCCAAGTTTTCTTTAAAATGAACAGTCTGCAATCTAATG
ACACAGCCATCTATTACTGTGCCAGAGCCCTCACCTACTATGATTACGAGTTTGCTT
ACTGGGGCCAAGGGACTCTGGTCACTGTCTCTGCAGGTGAGTCCTAACTTCAAGCTT
SEQ ID NO: 35
Amino acid sequence of 225 VH
MAVLALLFCLVTFPSCVLSQVQLKQSGPGLVQPSQSLSITCTVSGFSLTNYGV
HWVRQSPGKGLEWLGVIWSGGNTDYNTPFTSRLSINKDNSKSQVFFKMNSLQSNDTAI
YYCARALTYYDYEFAYWGQGTLVTVSA
SEQ ID NO: 36
DNA sequence of the 225 VL gene
GCTAGCGCCACCATGAGGGCCCCTGCTCAGTTTCTTGGCTTCTTGCTTTTC
TGGATTCCAGCCTCCAGAAGTGACATCTTGCTGACTCAGTCTCCAGTCATCCTGTCTG
TGAGTCCAGGAGAAAGAGTCAGTTTCTCCTGCAGGGCCAGTCAGAGTATTGGCACA
AACATCCACTGGTATCAGCAAAGAACAAATGGTTCTCCAAGGCTTCTCATAAAGTAT
GCTTCTGAGTCTATCTCTGGGATCCCTTCCAGGTTTAGTGGCAGTGGATCAGGGACA
GATTTTACTCTTAGCATCAACAGTGTGGAGTCTGAAGATATTGCAGATTATTACTGT
CAACAAAATAATAACTGGCCAACCACCTTCGGTGCTGGGACCAAGCTGGAGCTGAA
ACGTAAGTACACTTTTCTGAATTC
SEQ ID NO: 37
Amino acid sequence of 225 VL
MRAPAQFLGFLLFWIPASRSDILLTQSPVILSVSPGERVSFSCRASQSIGTNIHW
YQQRTNGSPRLLIKYASESISGIPSRFSGSGSGTDFTLSINSVESEDIADYYCQQNNNWPTT
FGAGTKLELK
SEQ ID NO: 38
Amino acid sequence of a mature heavy chain encoded in pJB559
QVQLKQSGPGLVQPSQSLSITCTVSGFSLTNYGVHWVRQSPGKGLEWLGVIW
SGGNTDYNTPFTSRLSINKDNSKSQVFFKMNSLQSNDTAIYYCARALTYYDYEFAYWG
QGTLVTVSAASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHTCP
PCPAPEAAGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGSGGGGSGGGGSEIVLT
QSPATLSLSPGERATLSCRSSTGAVTTSNYANWVQQKPGQAPRGLIGGTNKRAPGIPARF
SGSLSGTDATLTISSLEPEDFAVYYCALWYSNLWVFGCGTKVEIKGGGGSGGGGSGGG
GSEVQLVESGGGLVKPGGSLRLSCAASGFTFNTYAMNWVRQAPGKCLEWVARIRSKY
NNYATYYADSVKDRFTISRDDSKNTLYLQMNSLKTEDTAVYYCVRHGNFGNSYVSWF
AYWGQGTLVTVSS
SEQ ID NO: 39
Amino acid sequence of a mature light chain encoded in pJB559
DILLTQSPVILSVSPGERVSFSCRASQSIGTNIHWYQQRTNGSPRLLIKYASESIS
GIPSRFSGSGSGTDFTLSINSVESEDIADYYCQQNNNWPTTFGAGTKLELKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
SEQ ID NO: 40
Amino acid sequence of a mature 225 heavy chain fused to HuTEC1.scFv.ds encoded
in pYT419
QVQLKQSGPGLVQPSQSLSITCTVSGFSLTNYGVHWVRQSPGKGLEWLGVIW
SGGNTDYNTPFTSRLSINKDNSKSQVFFKMNSLQSNDTAIYYCARALTYYDYEFAYWG
QGTLVTVSAASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHTCP
PCPAPEAAGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGSGGGGSGGGGSDIVMT
QSPDSLAVSLGERATINCRASESVEYYGTSLMQWYQQKPGQPPKLLIYAASNVESGVPD
RFSGSGSGTDFTLTISSLQAEDVAVYYCQQSRKVPWTFGCGTKVEIKGGGGSGGGGSGG
GGSQVTLRESGPALVKPTQTLTLTCTFSGFSLTGYGIYWVRQPPGKCLEWLGMIWGSGS
TDYNSALRSRLTISKDTSKNQVVLTMTNMDPVDTATYYCARDRGYYGSSSLYYYAMD
SWGQGTTVTVSS
SEQ ID NO: 41
DNA sequence of the C2B8 VH gene
ACTAGTACCACCATGGGTTGGAGCCTCATCTTGCTCTTCCTTGTCGCTGTT
GCTACCCGTGTCCTGTCCCAAGTCCAACTGCAGCAGCCTGGGGCTGAGCTCGTGAAG
CCTGGGGCCTCAGTGAAGATGTCCTGCAAGGCTTCTGGCTACACATTTACCAGTTAC
AATATGCACTGGGTGAAACAGACACCTGGACGGGGCCTGGAATGGATTGGAGCTAT
TTATCCCGGAAATGGAGATACTTCCTACAATCAGAAGTTCAAAGGCAAGGCCACATT
GACTGCAGACAAATCCTCCAGCACAGCCTACATGCAGCTCAGCAGCCTGACATCTG
AGGACTCTGCCGTCTATTACTGTGCAAGATCCACTTACTACGGCGGCGACTGGTACT
TCAATGTCTGGGGCGCAGGGACCACTGTCACCGTCTCTGCAGGTGAGTCCTAACTTC
TCCAAGCTT
SEQ ID NO: 42
Amino acid sequence of C2B8 VH
MGWSLILLFLVAVATRVLSQVQLQQPGAELVKPGASVKMSCKASGYTFTSY
NMHWVKQTPGRGLEWIGAIYPGNGDTSYNQKFKGKATLTADKSSSTAYMQLSSLTSED
SAVYYCARSTYYGGDWYFNVWGAGTTVTVSA
SEQ ID NO: 43
Amino acid sequence of mature C2B8 VH
QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARSTYYGGDWYF
NVWGAGTTVTVSA
SEQ ID NO: 44
DNA sequence of the C2B8 VL gene
GCTAGCACCACCATGGATTTTCAAGTGCAGATTATCAGCTTCCTGCTGATC
AGTGCTTCAGTCATCATGTCCAGAGGACAAATTGTTCTCTCCCAGTCTCCAGCAATC
CTGTCTGCATCTCCAGGGGAGAAAGTCACAATGACTTGCAGGGCCAGCTCAAGTGT
GAGTTACATCCACTGGTTCCAGCAGAAGCCAGGATCCTCCCCCAAACCCTGGATTTA
TGCCACATCCAACCTGGCTTCTGGAGTCCCTGTTCGCTTCAGTGGCAGTGGCTCTGG
GACTTCTTACTCTCTCACAATCAGCAGAGTGGAGGCTGAAGATGCTGCCACTTATTA
CTGCCAGCAGTGGACCAGTAACCCACCCACCTTCGGAGGTGGCACCAAGCTGGAAA
TCAAACGTAAGTAGAATCCAAAGTGAATTC
SEQ ID NO: 45
Amino acid sequence of C2B8 VL
MDFQVQIISFLLISASVIMSRGQIVLSQSPAILSASPGEKVTMTCRASSSVSYIH
WFQQKPGSSPKPWIYATSNLASGVPVRFSGSGSGTSYSLTISRVEAEDAATYYCQQWTS
NPPTFGGGTKLEIK
SEQ ID NO: 46
Amino acid sequence of mature C2B8 VL
QIVLSQSPAILSASPGEKVTMTCRASSSVSYIHWFQQKPGSSPKPWIYATSNLA
SGVPVRFSGSGSGTSYSLTISRVEAEDAATYYCQQWTSNPPTFGGGTKLEIK
SEQ ID NO: 47
Amino acid sequence of a mature heavy chain encoded in pJB554
QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARSTYYGGDWYF
NVWGAGTTVTVSAASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKKVEPKSCDK
THTCPPCPAPEAAGGPSVFLFPPKPKDTLMISRTPEVTCVVVDVSHEDPEVKENWYVDG
VEVHNAKTKPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGSGGGGSGGGG
SEIVLTQSPATLSLSPGERATLSCRSSTGAVTTSNYANWVQQKPGQAPRGLIGGTNKRAP
GIPARFSGSLSGTDATLTISSLEPEDFAVYYCALWYSNLWVFGCGTKVEIKGGGGSGGG
GSGGGGSEVQLVESGGGLVKPGGSLRLSCAASGFTFNTYAMNWVRQAPGKCLEWVAR
IRSKYNNYATYYADSVKDRFTISRDDSKNTLYLQMNSLKTEDTAVYYCVRHGNFGNSY
VSWFAYWGQGTLVTVSS
SEQ ID NO: 48
Amino acid sequence of a mature light chain encoded in pJB554 and pYT405
QIVLSQSPAILSASPGEKVTMTCRASSSVSYIHWFQQKPGSSPKPWIYATSNLA
SGVPVRFSGSGSGTSYSLTISRVEAEDAATYYCQQWTSNPPTFGGGTKLEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
SEQ ID NO: 49
Amino acid sequence of a mature C2B8 heavy chain fused to HuTEC1.scFv.ds
encoded in pYT405
QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARSTYYGGDWYF
NVWGAGTTVTVSAASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKKVEPKSCDK
THTCPPCPAPEAAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGSGGGGSGGGG
SDIVMTQSPDSLAVSLGERATINCRASESVEYYGTSLMQWYQQKPGQPPKLLIYAASNV
ESGVPDRFSGSGSGTDFTLTISSLQAEDVAVYYCQQSRKVPWTFGCGTKVEIKGGGGSG
GGGSGGGGSQVTLRESGPALVKPTQTLTLTCTFSGFSLTGYGIYWVRQPPGKCLEWLG
MIWGSGSTDYNSALRSRLTISKDTSKNQVVLTMTNMDPVDTATYYCARDRGYYGSSSL
YYYAMDSWGQGTTVTVSS
SEQ ID NO: 50
Amino acid sequence of CDR H1 of SP34, HuSP34V and HuSP34V.scFv.ds
TYAMN
SEQ ID NO: 51:
Amino acid sequence of CDR H2 of SP34, HuSP34V and HuSP34V.scFv.ds
RIRSKYNNYATYYADSVKD
SEQ ID NO: 52
Amino acid sequence of CDR H3 of SP34, HuSP34V and HuSP34V.scFv.ds
HGNFGNSYVSWFAY
SEQ ID NO: 53
Amino acid sequence of CDR L1 of SP34, HuSP34V and HuSP34V.scFv.ds
RSSTGAVTTSNYAN
SEQ ID NO: 54
Amino acid sequence of CDR L2 of SP34, HuSP34V and HuSP34V.scFv.ds
GTNKRAP
SEQ ID NO: 55
Amino acid sequence of CDR L3 of SP34, HuSP34V and HuSP34V.scFv.ds
ALWYSNLWV
SEQ ID NO: 56
Amino acid sequence of mature SP34 VH
EVQLVESGGGLVQPKGSLKLSCAASGFTFNTYAMNWVRQAPGKGLEWVARI
RSKYNNYATYYADSVKDRFTISRDDSQSILYLQMNNLKTEDTAMYYCVRHGNFGNSYV
SWFAYWGQGTLVTVSS
SEQ ID NO: 57
Amino acid sequence of mature SP34 VL
QAVVTQESALTTSPGETVTLTCRSSTGAVTTSNYANWVQEKPDHLFTGLIGG
TNKRAPGVPARFSGSLIGDKAALTITGAQTEDEAIYFCALWYSNLWVFGGGTKLTVL
SEQ ID NO: 58
Amino acid sequence of mature HuSP34V VH
EVQLVESGGGLVKPGGSLRLSCAASGFTFNTYAMNWVRQAPGKGLEWVARI
RSKYNNYATYYADSVKDRFTISRDDSKNTLYLQMNSLKTEDTAVYYCVRHGNFGNSY
VSWFAYWGQGTLVTVSS
SEQ ID NO: 59
Amino acid sequence of mature HuSP34V VL
EIVLTQSPATLSLSPGERATLSCRSSTGAVTTSNYANWVQQKPGQAPRGLIGG
TNKRAPGIPARFSGSLSGTDATLTISSLEPEDFAVYYCALWYSNLWVFGGGTKVEIK
SEQ ID NO: 60
Amino acid sequence of CDR H1 of HuM195
DYNMH
SEQ ID NO: 61:
Amino acid sequence of CDR H2 of HuM195
YIYPYNGGTGYNQKFKS
SEQ ID NO: 62
Amino acid sequence of CDR H3 of HuM195
GRPAMDY
SEQ ID NO: 63
Amino acid sequence of CDR L1 of HuM195
RASESVDNYGISFMN
SEQ ID NO: 64
Amino acid sequence of CDR L2 of HuM195
AASNQGS
SEQ ID NO: 65
Amino acid sequence of CDR L3 of HuM195
QQSKEVPWT
SEQ ID NO: 66
Amino acid sequence of CDR H1 of 225
NYGVH
SEQ ID NO: 67:
Amino acid sequence of CDR H2 of 225
VIWSGGNTDYNTPFTS
SEQ ID NO: 68
Amino acid sequence of CDR H3 of 225
ALTYYDYEFAY
SEQ ID NO: 69
Amino acid sequence of CDR L1 of 225
RASQSIGTNIH
SEQ ID NO: 70
Amino acid sequence of CDR L2 of 225
YASESIS
SEQ ID NO: 71
Amino acid sequence of CDR L3 of 225
QQNNNWPTT
SEQ ID NO: 72
Amino acid sequence of CDR H1 of C2B8
SYNMH
SEQ ID NO: 73
Amino acid sequence of CDR H2 of C2B8
AIYPGNGDTSYNQKFKG
SEQ ID NO: 74
Amino acid sequence of CDR H3 of C2B8
STYYGGDWYFNV
SEQ ID NO: 75
Amino acid sequence of CDR L1 of C2B8
RASSSVSYIH
SEQ ID NO: 76
Amino acid sequence of CDR L2 of C2B8
ATSNLAS
SEQ ID NO: 77
Amino acid sequence of CDR L3 of C2B8
QQWTSNPPT
SEQ ID NO: 78
Amino acid sequence of mature TEC1 VH
QVQLKESGPGLVAPSQSLSITCTVSGFSLTGYGIYWVRQPPGKGLEWLGMIW
GDGSTDYNSALRSRLSINKDNSKSQVFLKMNSLQTDDTARYYCARDRGYYGSSSLYYY
AMDSWGQGTSVTVSS
SEQ ID NO: 79
Amino acid sequence of mature TEC1 VL
DIVLTQSPASLAVSLGQRATISCRASESVEYYGTSLMQWYQQKPGQPPKLLIY
AASNVESGVPARFSGSGSGTDFSLNIHPVEEDDIAMYFCQQSRKVPWTFGGGTKLEIK
SEQ ID NO: 80
Amino acid sequence of a mature 225 heavy chain fused to HuTEC1.scFv.ds with the
hole mutation encoded in pYT437
QVQLKQSGPGLVQPSQSLSITCTVSGFSLTNYGVHWVRQSPGKGLEWLGVIW
SGGNTDYNTPFTSRLSINKDNSKSQVFFKMNSLQSNDTAIYYCARALTYYDYEFAYWG
QGTLVTVSAASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHTCP
PCPAPEAAGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPR
EPQVYTLPPSRDELTKNQVSLSCAVKGFYPSDIAVEWESNGQPENNYKTTPPVLDSDGSF
FLVSKLTVDKSRWQQGNVFSCSVMHEALHNHYTQKSLSLSPGSGGGGSGGGGSDIVMT
QSPDSLAVSLGERATINCRASESVEYYGTSLMQWYQQKPGQPPKLLIYAASNVESGVPD
RFSGSGSGTDFTLTISSLQAEDVAVYYCQQSRKVPWTFGCGTKVEIKGGGGSGGGGSGG
GGSQVTLRESGPALVKPTQTLTLTCTFSGFSLTGYGIYWVRQPPGKCLEWLGMIWGSGS
TDYNSALRSRLTISKDTSKNQVVLTMTNMDPVDTATYYCARDRGYYGSSSLYYYAMD
SWGQGTTVTVSS
SEQ ID NO: 81
Amino acid sequence of a mature 225 heavy chain fused to HuSP34V.scFv.ds with
the knob mutation encoded in pYT437
QVQLKQSGPGLVQPSQSLSITCTVSGFSLTNYGVHWVRQSPGKGLEWLGVIW
SGGNTDYNTPFTSRLSINKDNSKSQVFFKMNSLQSNDTAIYYCARALTYYDYEFAYWG
QGTLVTVSAASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCDKTHTCP
PCPAPEAAGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSF
FLWSKLTVDKSRWQQGNVFSCSVMHEALHNHYTQKSLSLSPGSGGGGSGGGGSEIVLT
QSPATLSLSPGERATLSCRSSTGAVTTSNYANWVQQKPGQAPRGLIGGTNKRAPGIPARF
SGSLSGTDATLTISSLEPEDFAVYYCALWYSNLWVFGCGTKVEIKGGGGSGGGGSGGG
GSEVQLVESGGGLVKPGGSLRLSCAASGFTFNTYAMNWVRQAPGKCLEWVARIRSKY
NNYATYYADSVKDRFTISRDDSKNTLYLQMNSLKTEDTAVYYCVRHGNFGNSYVSWF
AYWGQGTLVTVSS
<SEQ ID NO: 82
Amino acid sequence of a linker
GGAA
<SEQ ID NO: 83
Amino acid sequence of a linker
GGGGS
<SEQ ID NO: 84
Amino acid sequence of a linker
LAAAA

1. A trispecific antibody comprising first and second heavy chains and first and second instances of a light chain, wherein:
(a) the light chain comprises a light chain variable region and a light chain constant region;
(b) the first heavy chain comprises a first heavy chain variable region, a first heavy chain constant region and an scFv comprising a second heavy chain variable region and a second light chain variable region;
(c) the second heavy chain comprises the first heavy chain variable region, a second heavy chain constant region and an scFv comprising a third heavy chain variable region and a third light chain variable region;
(d) the first and second heavy chains respectively associate with the first and second instances of the light chain to form two instances of a first binding site, each instance comprising the first heavy chain variable region and first light chain variable region, the first binding site specifically binding to a target antigen;
(e) the scFv comprising the second heavy chain variable region and second light chain variable region forms a second binding site specifically binding to CD3;
(f) the scFv comprising the third heavy chain variable region and third light chain variable region forms a third binding site specifically binding to CD28; and
(g) the first and second heavy chains associate with one another via the first and second constant regions to form a tetramer of the first and second heavy 1. A trispecific antibody comprising first and second heavy chains and first and second instances of a light chain, wherein:
(a) the light chain comprises a light chain variable region and a light chain constant region;
(b) the first heavy chain comprises a first heavy chain variable region, a first heavy chain constant region and an scFv comprising a second heavy chain variable region and a second light chain variable region;
(c) the second heavy chain comprises the first heavy chain variable region, a second heavy chain constant region and an scFv comprising a third heavy chain variable region and a third light chain variable region;
(d) the first and second heavy chains respectively associate with the first and second instances of the light chain to form two instances of a first binding site, each instance comprising the first heavy chain variable region and first light chain variable region, the first binding site specifically binding to a target antigen;
(e) the scFv comprising the second heavy chain variable region and second light chain variable region forms a second binding site specifically binding to CD3;
(f) the scFv comprising the third heavy chain variable region and third light chain variable region forms a third binding site specifically binding to CD28; and
(g) the first and second heavy chains associate with one another via the first and second constant regions to form a tetramer of the first and second heavy chains and the first and second instances of the light chain.

2. The trispecific antibody of claim 1, wherein the target is a cancer-associated antigen.

3. The trispecific antibody of claim 1, wherein the target is an antigen on an infected cell.

4. The trispecific antibody of claim 1, wherein the target is an antigen on an autoreactive cell.

5. The trispecific antibody of claim 1, wherein the first binding site antagonizes binding of the target to a ligand.

6. The trispecific antibody of claim 1, wherein the third heavy chain variable region comprises CDR H1 of SEQ ID NO:3, CDR H2 of SEQ ID NO:4 or 12 and CDR H3 of SEQ ID NO:5, and the third light chain variable region comprises CDRs L1, L2 and L3 of SEQ ID NOS: 7-9 respectively.

7. The trispecific antibody of claim 6, wherein the third heavy chain variable region and the third light chain variable region comprise SEQ ID NOS: 78 and 79 respectively, or SEQ ID NOS: 11 and 14 respectively.

8. The trispecific antibody of claim 1, wherein the scFv forming the third binding site against CD28 comprises SEQ ID NO:28.

9. The trispecific antibody of claim 1, wherein the second heavy chain variable region comprises CDRs H1, H2 and H3 of SEQ ID NOS: 50-52 respectively and the second light chain variable region comprises CDRs L1, L2 and L3 of SEQ ID NOS: 53-55 respectively.

10. The trispecific antibody of claim 1, wherein the scFv forming the second binding site against CD3 comprises SEQ ID NO:32.

11. The trispecific antibody of claim 1, wherein the first heavy chain variable region and first light chain variable region comprise residues 20-135 of SEQ ID NO:25 and residues 21-131 of SEQ ID NO:27 respectively.

12. The trispecific antibody of claim 1, wherein the first heavy chain variable region and first light chain variable region comprise residues 20-138 of SEQ ID NO:35 and residues 21-127 of SEQ ID NO:37 respectively.

13. The trispecific antibody of claim 1, wherein the first heavy chain variable region and the first light chain variable region comprises SEQ ID NO:43 and SEQ ID NO:46 respectively.

14. The trispecific antibody of claim 1, wherein the scFv forming the second binding site and the scFv forming the third binding site are linked via their light chain variable regions to the first and second heavy chain constant regions.

15. The trispecific antibody of claim 1, wherein the first and second heavy chains comprise SEQ ID NOS:30 and 33 respectively and the light chain comprises SEQ ID NO:31.

16. The trispecific antibody of claim 1, wherein the first, second and third binding sites are humanized, veneered or human and the heavy and light chain constant regions are human.

17. The trispecific antibody of claim 1, wherein the isotype of the first and second heavy chain constant regions is human IgG1 and the light chain constant region is human kappa.

18. The trispecific antibody of claim 1, wherein one or both of the heavy chain constant regions have at least one mutation reducing FcRγ binding.

19. The trispecific antibody of claim 1, wherein one or both of the heavy chain constant regions have at least one mutation increasing binding to FcRn.

20. The trispecific antibody of claim 1, wherein one of the first heavy chain constant region and second heavy chain constant region has at least one knob mutation and the other has at least one hole mutation to facilitate association of the first and second heavy chain constant regions.

21. A monoclonal antibody specifically binding to CD28 comprising a heavy chain variable region comprising CDR H1 of SEQ ID NO:3, CDR H2 of SEQ ID NO:4 or 12 and CDR H3 of SEQ ID NO:5, and a light chain variable region comprises CDRs L1, L2 and L3 of SEQ ID NOS: 7-9 respectively.

22. The monoclonal antibody of claim 21, comprising heavy and light chain variable regions of SEQ ID NOS: 78 and 79 respectively, or SEQ ID NOS: 11 and 14 respectively.

23. The monoclonal antibody of claim 21 in the form of an scFv.

24. The monoclonal antibody of claim 23 comprising SEQ ID NO:28.

25. The monoclonal antibody of claim 2, wherein the first heavy chain variable region is linked to a heavy chain constant region and the first light chain variable region is linked to a light chain constant region.

26. The monoclonal antibody of claim 25 in the form of a tetramer comprising two instances of the heavy chain and two instances of the light chain.

27. The monoclonal antibody of claim 21, wherein the heavy chain variable region and the light chain variable region form a first binding site and the antibody further comprises a second binding site for a target other than CD28.

28. The monoclonal antibody of claim 27, wherein the target other than CD28 is a cancer-associated antigen, an antigen on an infected cell or an antigen on an autoreactive cell.

29. A pharmaceutical composition comprising a trispecific antibody or monoclonal antibody of claim 1 and a pharmaceutically acceptable carrier.

30. A method of treating or effecting prophylaxis of cancer, comprising administering an effective regime of a trispecific antibody as defined in claim 1 to a subject having or at risk of cancer.

31. The method of claim 30, wherein the cancer expresses the target antigen.

32. A method of treating an infection, comprising administering an effective regime of a trispecific antibody as defined by claim 1 to a subject having or at risk of infection.

33. The method of claim 32, wherein the subject has infected cells comprising the target antigen.


SEQUENCE LISTING (84 sequences)

SEQ ID NO: 1 - protein, 134 aa, Homo sapiens
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKP

SEQ ID NO: 2 - protein, 144 aa, Synthetic construct
MAVLALLFCLVTFPSCILSQVQLKESGPGLVAPSQSLSITCTVSGFSLTGYGIYWVRQPPGKGLEWLGMIWGDGSTDYNSALRSRLSINKDNSKSQVFLKMNSLQTDDTARYYCARDRGYYGSSSLYYYAMDSWGQGTSVTVSS

SEQ ID NO: 3 - protein, 5 aa, Synthetic construct
GYGIY

SEQ ID NO: 4 - protein, 16 aa, Synthetic construct
MIWGDGSTDYNSALRS

SEQ ID NO: 5 - protein, 17 aa, Synthetic construct
DRGYYGSSSLYYYAMDS

SEQ ID NO: 6 - protein, 131 aa, Synthetic construct
MESDTLLLWVLLLWVPGSTGDIVLTQSPASLAVSLGQRATISCRASESVEYYGTSLMQWYQQKPGQPPKLLIYAASNVESGVPARFSGSGSGTDFSLNIHPVEEDDIAMYFCQQSRKVPWTFGGGTKLEIK

SEQ ID NO: 7 - protein, 15 aa, Synthetic construct
RASESVEYYGTSLMQ

SEQ ID NO: 8 - protein, 7 aa, Synthetic construct
AASNVES

SEQ ID NO: 9 - protein, 9 aa, Synthetic construct
QQSRKVPWT

SEQ ID NO: 10 - protein, 144 aa, Synthetic construct
MAVLALLFCLVTFPSCILSQVTLRESGPALVKPTQTLTLTCTFSGFSLTGYGIYWVRQPPGKALEWLGMIWGSGSTDYNSALRSRLTISKDTSKNQVVLTMTNMDPVDTATYYCARDRGYYGSSSLYYYAMDSWGQGTTVTVSS

SEQ ID NO: 11 - protein, 125 aa, Synthetic construct
QVTLRESGPALVKPTQTLTLTCTFSGFSLTGYGIYWVRQPPGKALEWLGMIWGSGSTDYNSALRSRLTISKDTSKNQVVLTMTNMDPVDTATYYCARDRGYYGSSSLYYYAMDSWGQGTTVTVSS

SEQ ID NO: 12 - protein, 16 aa, Synthetic construct
MIWGSGSTDYNSALRS

SEQ ID NO: 13 - protein, 131 aa, Synthetic construct
MESDTLLLWVLLLWVPGSTGDIVMTQSPDSLAVSLGERATINCRASESVEYYGTSLMQWYQQKPGQPPKLLIYAASNVESGVPDRFSGSGSGTDFTLTISSLQAEDVAVYYCQQSRKVPWTFGGGTKVEIK

SEQ ID NO: 14 - protein, 111 aa, Synthetic construct
DIVMTQSPDSLAVSLGERATINCRASESVEYYGTSLMQWYQQKPGQPPKLLIYAASNVESGVPDRFSGSGSGTDFTLTISSLQAEDVAVYYCQQSRKVPWTFGGGTKVEIK

SEQ ID NO: 15 - DNA, 467 bp, Synthetic construct
  source 1..467: mol_type = other DNA
actagtaccaccatggctgtcctggcacttctcttctgcctggtcacattcccaagctgtatcctttcccaagtcactctgagggagtcaggacctgcccttgtgaagcccacacagaccctgaccctcacatgcaccttctcagggttctcactgaccggctatggaatctactgggttcgccagcctccaggaaaggctctggagtggctgggaatgatctggggaagtggaagcacagactataattcagctctcagatccagactgaccatcagcaaggacacctccaagaaccaagttgtcctgacaatgaccaatatggaccctgttgacacagccacctactactgtgccagagatcggggatactacggaagtagctccctgtattactatgctatggactcctggggacaaggaaccacagtcaccgtctcctcaggtaagtatggcctctcaagctt

SEQ ID NO: 16 - DNA, 428 bp, Synthetic construct
  source 1..428: mol_type = other DNA
gctagcaccaccatggagtcagacacactcctgctttgggttctgctgctctgggttccaggctccactggagacattgtgatgacccaatctccagattctttggctgtgtctcttggcgaaagagccaccatcaactgcagagccagtgaaagtgttgaatactatggcacaagtctgatgcagtggtaccaacagaaaccaggacagccacccaaactcctcatctatgctgcatccaatgtcgaatctggggtccctgacagatttagtggcagtggctctgggacagacttcaccctcaccatcagttctctgcaggccgaggatgttgcagtgtattactgtcagcaaagtaggaaggttccatggaccttcggaggaggcaccaaagttgaaatcaaacgtaagtagaatccaaagaattc

SEQ ID NO: 17 - protein, 98 aa, Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 18 - protein, 15 aa, Synthetic construct
EPKSCDKTHTCPPCP

SEQ ID NO: 19 - protein, 110 aa, Synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 20 - protein, 107 aa, Synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 107 aa, Synthetic construct [sequence omitted]

SEQ ID NO: 22 - protein, 455 aa, Synthetic construct
QVTLRESGPALVKPTQTLTLTCTFSGFSLTGYGIYWVRQPPGKALEWLGMIWGSGSTDYNSALRSRLTISKDTSKNQVVLTMTNMDPVDTATYYCARDRGYYGSSSLYYYAMDSWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 218 aa, Synthetic construct
DIVMTQSPDSLAVSLGERATINCRASESVEYYGTSLMQWYQQKPGQPPKLLIYAASNVESGVPDRFSGSGSGTDFTLTISSLQAEDVAVYYCQQSRKVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - DNA, 441 bp, Synthetic construct
  source 1..441: mol_type = other DNA
actagtaccaccatgggatggagctggatctttttcttcctcctgtcaggaactgccagcgtcctgtcccaagtccagcttgtgcagtcaggagctgaagtcaagaaacctgggtcctcagtgaaagtctcctgcaaggcttctggatacacattcactgactacaacatgcactgggtccgacaggcccctggacagggcctcgagtggattggatatatttatccttacaatggcgggactggctacaaccagaagttcaagagcaaggccacaatcactgcagacgaatccaccaacacagcctacatggaactcagcagcctgagatctgaggacactgcagtctattactgtgcaagagggcgacccgctatggactactggggtcaaggaaccctggtcaccgtctcctcaggtaagaatggcctctccaagctt

SEQ ID NO: 25 - protein, 135 aa, Synthetic construct
MGWSWIFFFLLSGTASVLSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 26 - DNA, 430 bp, Synthetic construct
  source 1..430: mol_type = other DNA
gctagcaccaccatggagaaagacacactcctgctgtgggtcctgcttctctgggttccagggtccacaggcgacattcagatgacccaatctccatcttctttgtctgcctctgtgggggacagggtcaccatcacctgcagagccagcgaaagtgttgataattatggcattagttttatgaactggttccaacagaaaccaggaggggcacccaaactcctcatctatgctgcatccaaccaagggtccggggtcccttccaggtttagtggcagtggctctgggacagacttcaccctcaccatctcttctctgcagcccgatgattttgcaacctattactgtcagcaaagtaaggaagttccctggaccttcgggcagggcaccaaagtcgaaatcaaacgtaagtagaatccaaagtgaattc

SEQ ID NO: 27 - protein, 131 aa, Synthetic construct
MEKDTLLLWVLLLWVPGSTGDIQMTQSPSSLSASVGDRVTITCRASESVDNYGISFMNWFQQKPGGAPKLLIYAASNQGSGVPSRFSGSGSGTDFTLTISSLQPDDFATYYCQQSKEVPWTFGQGTKVEIK

SEQ ID NO: 28 - protein, 251 aa, Synthetic construct
DIVMTQSPDSLAVSLGERATINCRASESVEYYGTSLMQWYQQKPGQPPKLLIYAASNVESGVPDRFSGSGSGTDFTLTISSLQAEDVAVYYCQQSRKVPWTFGCGTKVEIKGGGGSGGGGSGGGGSQVTLRESGPALVKPTQTLTLTCTFSGFSLTGYGIYWVRQPPGKCLEWLGMIWGSGSTDYNSALRSRLTISKDTSKNQVVLTMTNMDPVDTATYYCARDRGYYGSSSLYYYAMDSWGQGTTVTVSS

SEQ ID NO: 29 - protein, 368 aa, Synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSDIVMTQSPDSLAVSLGERATINCRASESVEYYGTSLMQWYQQKPGQPPKLLIYAASNVESGVPDRFSGSGSGTDFTLTISSLQAEDVAVYYCQQSRKVPWTFGCGTKVEIKGGGGSGGGGSGGGGSQVTLRESGPALVKPTQTLTLTCTFSGFSLTGYGIYWVRQPPGKCLEWLGMIWGSGSTDYNSALRSRLTISKDTSKNQVVLTMTNMDPVDTATYYCARDRGYYGSSSLYYYAMDSWGQGTTVTVSS

SEQ ID NO: 30 - protein, 707 aa, Synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSDIVMTQSPDSLAVSLGERATINCRASESVEYYGTSLMQWYQQKPGQPPKLLIYAASNVESGVPDRFSGSGSGTDFTLTISSLQAEDVAVYYCQQSRKVPWTFGCGTKVEIKGGGGSGGGGSGGGGSQVTLRESGPALVKPTQTLTLTCTFSGFSLTGYGIYWVRQPPGKCLEWLGMIWGSGSTDYNSALRSRLTISKDTSKNQVVLTMTNMDPVDTATYYCARDRGYYGSSSLYYYAMDSWGQGTTVTVSS

SEQ ID NO: 31 - protein, 218 aa, Synthetic construct
DIQMTQSPSSLSASVGDRVTITCRASESVDNYGISFMNWFQQKPGGAPKLLIYAASNQGSGVPSRFSGSGSGTDFTLTISSLQPDDFATYYCQQSKEVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 250 aa, Synthetic construct
EIVLTQSPATLSLSPGERATLSCRSSTGAVTTSNYANWVQQKPGQAPRGLIGGTNKRAPGIPARFSGSLSGTDATLTISSLEPEDFAVYYCALWYSNLWVFGCGTKVEIKGGGGSGGGGSGGGGSEVQLVESGGGLVKPGGSLRLSCAASGFTFNTYAMNWVRQAPGKCLEWVARIRSKYNNYATYYADSVKDRFTISRDDSKNTLYLQMNSLKTEDTAVYYCVRHGNFGNSYVSWFAYWGQGTLVTVSS

SEQ ID NO: 33 - protein, 706 aa, Synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSEIVLTQSPATLSLSPGERATLSCRSSTGAVTTSNYANWVQQKPGQAPRGLIGGTNKRAPGIPARFSGSLSGTDATLTISSLEPEDFAVYYCALWYSNLWVFGCGTKVEIKGGGGSGGGGSGGGGSEVQLVESGGGLVKPGGSLRLSCAASGFTFNTYAMNWVRQAPGKCLEWVARIRSKYNNYATYYADSVKDRFTISRDDSKNTLYLQMNSLKTEDTAVYYCVRHGNFGNSYVSWFAYWGQGTLVTVSS

SEQ ID NO: 34 - DNA, 448 bp, Synthetic construct
  source 1..448: mol_type = other DNA
actagtgccaccatggctgtcttggccctgctcttctgcctggtgacattcccaagctgtgtcctgtcccaggtgcagctgaagcagtcaggacctggcctagtgcagccctcacagagcctgtccatcacctgcacagtctctggtttctcactgactaactatggtgtgcactgggttcgccagtctccaggaaagggtctggagtggctgggagtgatctggagtggtggaaacacagactataatacacctttcacatccagactgagcatcaacaaggacaattccaagagccaagttttctttaaaatgaacagtctgcaatctaatgacacagccatctattactgtgccagagccctcacctactatgattacgagtttgcttactggggccaagggactctggtcactgtctctgcaggtgagtcctaacttcaagctt

SEQ ID NO: 35 - protein, 138 aa, Synthetic construct
MAVLALLFCLVTFPSCVLSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 36 - DNA, 416 bp, Synthetic construct
  source 1..416: mol_type = other DNA
gctagcgccaccatgagggcccctgctcagtttcttggcttcttgcttttctggattccagcctccagaagtgacatcttgctgactcagtctccagtcatcctgtctgtgagtccaggagaaagagtcagtttctcctgcagggccagtcagagtattggcacaaacatccactggtatcagcaaagaacaaatggttctccaaggcttctcataaagtatgcttctgagtctatctctgggatcccttccaggtttagtggcagtggatcagggacagattttactcttagcatcaacagtgtggagtctgaagatattgcagattattactgtcaacaaaataataactggccaaccaccttcggtgctgggaccaagctggagctgaaacgtaagtacacttttctgaattc

SEQ ID NO: 37 - protein, 127 aa, Synthetic construct
MRAPAQFLGFLLFWIPASRSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 38 - protein, 709 aa, Synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSEIVLTQSPATLSLSPGERATLSCRSSTGAVTTSNYANWVQQKPGQAPRGLIGGTNKRAPGIPARFSGSLSGTDATLTISSLEPEDFAVYYCALWYSNLWVFGCGTKVEIKGGGGSGGGGSGGGGSEVQLVESGGGLVKPGGSLRLSCAASGFTFNTYAMNWVRQAPGKCLEWVARIRSKYNNYATYYADSVKDRFTISRDDSKNTLYLQMNSLKTEDTAVYYCVRHGNFGNSYVSWFAYWGQGTLVTVSS

SEQ ID NO: 39 - protein, 214 aa, Synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 710 aa, Synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSDIVMTQSPDSLAVSLGERATINCRASESVEYYGTSLMQWYQQKPGQPPKLLIYAASNVESGVPDRFSGSGSGTDFTLTISSLQAEDVAVYYCQQSRKVPWTFGCGTKVEIKGGGGSGGGGSGGGGSQVTLRESGPALVKPTQTLTLTCTFSGFSLTGYGIYWVRQPPGKCLEWLGMIWGSGSTDYNSALRSRLTISKDTSKNQVVLTMTNMDPVDTATYYCARDRGYYGSSSLYYYAMDSWGQGTTVTVSS

SEQ ID NO: 41 - DNA, 457 bp, Synthetic construct
  source 1..457: mol_type = other DNA
actagtaccaccatgggttggagcctcatcttgctcttccttgtcgctgttgctacccgtgtcctgtcccaagtccaactgcagcagcctggggctgagctcgtgaagcctggggcctcagtgaagatgtcctgcaaggcttctggctacacatttaccagttacaatatgcactgggtgaaacagacacctggacggggcctggaatggattggagctatttatcccggaaatggagatacttcctacaatcagaagttcaaaggcaaggccacattgactgcagacaaatcctccagcacagcctacatgcagctcagcagcctgacatctgaggactctgccgtctattactgtgcaagatccacttactacggcggcgactggtacttcaatgtctggggcgcagggaccactgtcaccgtctctgcaggtgagtcctaacttctccaagctt

SEQ ID NO: 42 - protein, 140 aa, Synthetic construct
MGWSLILLFLVAVATRVLSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSA

SEQ ID NO: 43 - protein, 121 aa, Synthetic construct [sequence omitted]

SEQ ID NO: 44 - DNA, 421 bp, Synthetic construct
  source 1..421: mol_type = other DNA
gctagcaccaccatggattttcaagtgcagattatcagcttcctgctgatcagtgcttcagtcatcatgtccagaggacaaattgttctctcccagtctccagcaatcctgtctgcatctccaggggagaaagtcacaatgacttgcagggccagctcaagtgtgagttacatccactggttccagcagaagccaggatcctcccccaaaccctggatttatgccacatccaacctggcttctggagtccctgttcgcttcagtggcagtggctctgggacttcttactctctcacaatcagcagagtggaggctgaagatgctgccacttattactgccagcagtggaccagtaacccacccaccttcggaggtggcaccaagctggaaatcaaacgtaagtagaatccaaagtgaattc

SEQ ID NO: 45 - protein, 128 aa, Synthetic construct
MDFQVQIISFLLISASVIMSRGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIK

SEQ ID NO: 46 - protein, 106 aa, Synthetic construct [sequence omitted]

SEQ ID NO: 47 - protein, 711 aa, Synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSEIVLTQSPATLSLSPGERATLSCRSSTGAVTTSNYANWVQQKPGQAPRGLIGGTNKRAPGIPARFSGSLSGTDATLTISSLEPEDFAVYYCALWYSNLWVFGCGTKVEIKGGGGSGGGGSGGGGSEVQLVESGGGLVKPGGSLRLSCAASGFTFNTYAMNWVRQAPGKCLEWVARIRSKYNNYATYYADSVKDRFTISRDDSKNTLYLQMNSLKTEDTAVYYCVRHGNFGNSYVSWFAYWGQGTLVTVSS

SEQ ID NO: 48 - protein, 213 aa, Synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 712 aa, Synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGSGGGGSDIVMTQSPDSLAVSLGERATINCRASESVEYYGTSLMQWYQQKPGQPPKLLIYAASNVESGVPDRFSGSGSGTDFTLTISSLQAEDVAVYYCQQSRKVPWTFGCGTKVEIKGGGGSGGGGSGGGGSQVTLRESGPALVKPTQTLTLTCTFSGFSLTGYGIYWVRQPPGKCLEWLGMIWGSGSTDYNSALRSRLTISKDTSKNQVVLTMTNMDPVDTATYYCARDRGYYGSSSLYYYAMDSWGQGTTVTVSS

SEQ ID NO: 50 - protein, 5 aa, Synthetic construct
TYAMN

SEQ ID NO: 51 - protein, 19 aa, Synthetic construct
RIRSKYNNYATYYADSVKD

SEQ ID NO: 52 - protein, 14 aa, Synthetic construct
HGNFGNSYVSWFAY

SEQ ID NO: 53 - protein, 14 aa, Synthetic construct
RSSTGAVTTSNYAN

SEQ ID NO: 54 - protein, 7 aa, Synthetic construct
GTNKRAP

SEQ ID NO: 55 - protein, 9 aa, Synthetic construct
ALWYSNLWV

SEQ ID NO: 56 - protein, 125 aa, Synthetic construct
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSS

SEQ ID NO: 57 - protein, 109 aa, Synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 58 - protein, 125 aa, Synthetic construct
EVQLVESGGGLVKPGGSLRLSCAASGFTFNTYAMNWVRQAPGKGLEWVARIRSKYNNYATYYADSVKDRFTISRDDSKNTLYLQMNSLKTEDTAVYYCVRHGNFGNSYVSWFAYWGQGTLVTVSS

SEQ ID NO: 59 - protein, 110 aa, Synthetic construct
EIVLTQSPATLSLSPGERATLSCRSSTGAVTTSNYANWVQQKPGQAPRGLIGGTNKRAPGIPARFSGSLSGTDATLTISSLEPEDFAVYYCALWYSNLWVFGGGTKVEIK

SEQ ID NO: 60 - protein, 5 aa, Synthetic construct
DYNMH

SEQ ID NO: 61 - protein, 17 aa, Synthetic construct
YIYPYNGGTGYNQKFKS

SEQ ID NO: 62 - protein, 7 aa, Synthetic construct
GRPAMDY

SEQ ID NO: 63 - protein, 15 aa, Synthetic construct
RASESVDNYGISFMN

SEQ ID NO: 64 - protein, 7 aa, Synthetic construct
AASNQGS

SEQ ID NO: 65 - protein, 9 aa, Synthetic construct
QQSKEVPWT

SEQ ID NO: 66 - protein, 5 aa, Synthetic construct
NYGVH

SEQ ID NO: 67 - protein, 16 aa, Synthetic construct
VIWSGGNTDYNTPFTS

SEQ ID NO: 68 - protein, 11 aa, Synthetic construct
ALTYYDYEFAY

SEQ ID NO: 69 - protein, 11 aa, Synthetic construct
RASQSIGTNIH

SEQ ID NO: 70 - protein, 7 aa, Synthetic construct
YASESIS

SEQ ID NO: 71 - protein, 9 aa, Synthetic construct
QQNNNWPTT

SEQ ID NO: 72 - protein, 5 aa, Synthetic construct
SYNMH

SEQ ID NO: 73 - protein, 17 aa, Synthetic construct
AIYPGNGDTSYNQKFKG

SEQ ID NO: 74 - protein, 12 aa, Synthetic construct
STYYGGDWYFNV

SEQ ID NO: 75 - protein, 10 aa, Synthetic construct
RASSSVSYIH

SEQ ID NO: 76 - protein, 7 aa, Synthetic construct
ATSNLAS

SEQ ID NO: 77 - protein, 9 aa, Synthetic construct
QQWTSNPPT

SEQ ID NO: 78 - protein, 125 aa, Synthetic construct
QVQLKESGPGLVAPSQSLSITCTVSGFSLTGYGIYWVRQPPGKGLEWLGMIWGDGSTDYNSALRSRLSINKDNSKSQVFLKMNSLQTDDTARYYCARDRGYYGSSSLYYYAMDSWGQGTSVTVSS

SEQ ID NO: 79 - protein, 111 aa, Synthetic construct
DIVLTQSPASLAVSLGQRATISCRASESVEYYGTSLMQWYQQKPGQPPKLLIYAASNVESGVPARFSGSGSGTDFSLNIHPVEEDDIAMYFCQQSRKVPWTFGGGTKLEIK

SEQ ID NO: 80 - protein, 710 aa, Synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGSGGGGSGGGGSDIVMTQSPDSLAVSLGERATINCRASESVEYYGTSLMQWYQQKPGQPPKLLIYAASNVESGVPDRFSGSGSGTDFTLTISSLQAEDVAVYYCQQSRKVPWTFGCGTKVEIKGGGGSGGGGSGGGGSQVTLRESGPALVKPTQTLTLTCTFSGFSLTGYGIYWVRQPPGKCLEWLGMIWGSGSTDYNSALRSRLTISKDTSKNQVVLTMTNMDPVDTATYYCARDRGYYGSSSLYYYAMDSWGQGTTVTVSS

SEQ ID NO: 81 - protein, 709 aa, Synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLWSKLTVDKSRWQQGNVFSCSVMHEALHNHYTQKSLSLSPGSGGGGSGGGGSEIVLTQSPATLSLSPGERATLSCRSSTGAVTTSNYANWVQQKPGQAPRGLIGGTNKRAPGIPARFSGSLSGTDATLTISSLEPEDFAVYYCALWYSNLWVFGCGTKVEIKGGGGSGGGGSGGGGSEVQLVESGGGLVKPGGSLRLSCAASGFTFNTYAMNWVRQAPGKCLEWVARIRSKYNNYATYYADSVKDRFTISRDDSKNTLYLQMNSLKTEDTAVYYCVRHGNFGNSYVSWFAYWGQGTLVTVSS

SEQ ID NO: 82 - protein, 4 aa, Synthetic construct
GGAA

SEQ ID NO: 83 - protein, 5 aa, Synthetic construct
GGGGS

SEQ ID NO: 84 - protein, 5 aa, Synthetic construct
LAAAA